(19) United States
(12) Patent Application Publication
(10) Pub. No.: US 2015/0299143 A1
(43) Pub. Date: Oct. 22, 2015

(54) SUBSTITUTED TRIAZOLE AND IMIDAZOLE COMPOUNDS

(71) Applicant: HOFFMANN-LA ROCHE INC., Nutley, NJ (US)

(72) Inventors: Stephen M. Lynch, Westfield, NJ (US); Rainer E. Martin, Basel (CH); Werner Neidhart, Basel (CH); Jean-Marc Plancher, Hagenthal-le-Bas (FR); Tanja Schulz-Gasch, Ziefen (CH)

(73) Assignee: F. HOFFMANN-LA ROCHE AG, Basel (CH)

(21) Appl. No.: 14/646,806

(22) PCT Filed: Nov. 29, 2013
(86) PCT No.: PCT/EP2013/075028

§ 371 Date: May 22, 2015

Related U.S. Application Data
(60) Provisional application No. 61/732,454, filed on Dec. 3, 2012.

(51) Int. Cl. C07D 249/04; C07D 409/12; C07D 403/06; C07D 405/12; C07D 401/12

(52) U.S. Cl. CPC C07D 249/04; C07D 405/12; C07D 401/12; C07D 403/06; C07D 409/12

(57) ABSTRACT

The invention is concerned with the compounds of formula (I): and pharmaceutically acceptable salts thereof. In addition, the present invention relates to methods of manufacturing and using the compounds of formula (I) as well as pharmaceutical compositions containing such compounds. The compounds of formula (I) are LMP7 inhibitors and may be useful in treating associated inflammatory diseases and disorders such as, for example, rheumatoid arthritis, lupus and irritable bowel disease.

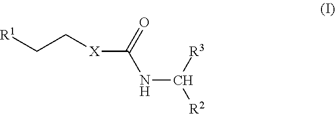

### Field of the Invention

The present invention relates to organic compounds useful for therapy and/or prophylaxis in a mammal of an inflammatory disease or disorder, and in particular to substituted triazole and imidazole compounds for the treatment of rheumatoid arthritis, lupus and irritable bowel disease (IBD), their manufacture, pharmaceutical compositions containing them and their use as LMP7 inhibitors.

### BACKGROUND OF THE INVENTION

LMP7 is an essential component of the immunoproteasome, mainly expressed in immune cells such as T/B lymphocytes and monocytes, as well as non-immune cells that have exposed to inflammatory cytokines, including IFN-γ and TNFα. Immunoproteasome plays an essential role in generation of antigenic peptide repertoire and shaping MHC class I restricted CD8+ T cell response. Moebius J. et al. European Journal of Immunology. 2010; Basler, M. et al. Journal of Immunology. 2004. 3925-34. Emerging data suggested that LMP7 also regulate inflammatory cytokine production and immune cell functions beyond the regulation of MHC class I mediated antigen presentation.

A small molecule LMP7 inhibitor, PR-957, has been shown to potently block Th1/17 differentiation, B cell effector functions and production of inflammatory cytokines (IL-6, TNF-α, IL-23). Muchamuel T. et al. Natural Medicine. 2009. 15, 781-787; Basler M. et al. Journal of Immunology. 2010, 634-41.

In addition, LMP7 blockade with PR-957 has been demonstrated to produce therapeutic benefits in several preclinical autoimmune disease models. First, PR-957 was demonstrated to significantly decrease disease score in mouse CAIA and CIA arthritis models, with hallmarks of significantly reduced inflammation and bone erosion. Muchamuel T. et al. Natural Medicine. 2009. 15, 781-787. In addition, PR-957 reduced plasma cells numbers and levels of anti-dsDNA IgG in MRL/lpr lupus-prone mice model, and prevented disease progression in these mice. Ichikawa H T, et al. Arthritis & Rheumatism. 2012. 64, 493-503. Furthermore, PR-957 reduced inflammation and tissue destruction in a DSS-induced colitis model in mice. Basler M. et al. Journal of Immunology. 2010, 634-41. Lastly, LMP7 knockout mice had also been shown to be protected from disease in IBD models. Schmidt N. et al. Gut 2010. 896-906.

Taken together, data strongly suggests that LMP7 activity is closely related to the functions of B/T lymphocytes and production of inflammatory cytokines, all of which are clinically validated targets/pathways in the pathogenesis of rheumatoid arthritis, lupus and IBD. Thus, existing data have provided strong rationale for targeting LMP7 for autoimmune disease indications. Due to potential liability with long term usage of a covalent inhibitor in chronic diseases like autoimmunity, a covalent reversible or non-covalent small molecule LMP7 inhibitor is highly desired for autoimmune disease indications.

### SUMMARY OF THE INVENTION

The invention provides for a compound of formula (I):

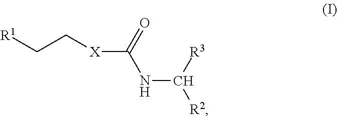

wherein:
X is triazole or imidazole;
R¹ is —NHC(═O)R⁴, —CH₂NHC(═O)R⁴, —NHC(═S)R⁴, —N═C(SCH₃)R⁴ or isoindolyl which is optionally substituted with oxo and/or C₁₋₇ alkoxy;
R² is —C(═O)OH, —C(═O)—C₁₋₇ alkoxy, —(CH₂)₂-phenyl, benzothiophenyl, naphthalenyl, benzodioxolyl, —C(═O)-methoxyphenyl, —CH₂C(═O)-phenyl, —(C(═O))₂NHCH₂-phenyl, phenyl or pyridinyl,
   wherein said pyridinyl is optionally mono-substituted with C₁₋₇ alkyl or C₁₋₇ alkoxy,
   wherein said phenyl is optionally mono- or bi-substituted independently with C₁₋₇ alkyl, —SCH₃, —CF₃, halogen, halo-C₁₋₇ alkyl, C₁₋₇ alkoxy, —OCF₃, —OCH₂-phenyl or cyclopropyl;
R³ is hydrogen, cyano, —CH₂-indolyl or C₁₋₇ alkyl, said C₁₋₇ alkyl optionally substituted with phenyl;
R⁴ is phenyl, pyridinyl, pyridazinyl optionally mono-substituted with C₁₋₇ alkoxy, indenyl optionally mono-substituted with C₁₋₇ alkyl, —(CH₂)ₙ-dihydroindenyl optionally mono-substituted with hydroxy wherein n is 0 or 1, dihydrobenzofuranyl, dihydrobenzodioxinyl, isoindolinyl, benzofuranyl, or benzodioxinyl,
   wherein said phenyl is optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl,
   wherein said pyridinyl is optionally mono- or bi-substituted independently with phenyl, C₁₋₇ alkoxy, —CF₃, —O-chlorophenyl, halogen or C₁₋₇ alkyl;
or a pharmaceutically acceptable salt thereof.

The invention also provides for pharmaceutical compositions comprising the compounds, methods of using the compounds and methods of preparing the compounds.

All documents cited to or relied upon are expressly incorporated herein by reference.

### DETAILED DESCRIPTION OF THE INVENTION

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule.

For example, the R variables of formula I refer to moieties that are attached to the core structure of formula I by a covalent bond.

In reference to a particular moiety with one or more hydrogen atoms, the term “substituted” refers to the fact that at least one of the hydrogen atoms of that moiety is replaced by another substituent or moiety. For example, the term “C₁₋₇ alkyl substituted by halogen” refers to the fact that one or more hydrogen atoms of a C₁₋₇ alkyl (as defined below) is replaced by one or more halogen atoms (e.g., trifluoromethyl, difluoromethyl, fluoromethyl, chloromethyl, etc.).

The term “alkyl” refers to an aliphatic straight-chain or branched-chain saturated hydrocarbon moiety having 1 to 20 carbon atoms. In particular embodiments the alkyl has 1 to 10 carbon atoms.

The term “C₁₋₇ alkyl” refers to an alkyl moiety having 1 to 7 carbon atoms. Examples of C1-7 alkyls include methyl, ethyl, propyl, isopropyl, butyl, isobutyl, sec-butyl and tert-butyl.

The term “C₁₋₇ alkoxy” denotes a group of the formula —O—R′, wherein R′ is an C₁₋₇ alkyl group. Examples of C₁₋₇ alkoxy moieties include methoxy, ethoxy, isopropoxy, and tert-butoxy.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

The terms “halo”, “halogen” and “halide”, which may be used interchangeably, refer to a substituent fluoro, chloro, bromo, or iodo.

Unless otherwise indicated, the term “hydrogen” or “hydro” refers to the moiety of a hydrogen atom (—H) and not H₂.

Unless otherwise indicated, the term “a compound of the formula” or “a compound of formula” or “compounds of the formula” or “compounds of formula” refers to any compound selected from the genus of compounds as defined by the formula (including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present invention can be present in the form of pharmaceutically acceptable salts. The compounds of the present invention can also be present in the form of pharmaceutically acceptable esters (i.e., the methyl and ethyl esters of the acids of formula I to be used as prodrugs). The compounds of the present invention can also be solvated, i.e. hydrated. The solvation can be effected in the course of the manufacturing process or can take place as a consequence of hygroscopic properties of an initially anhydrous compound of formula I (hydration).

Compounds that have the same molecular formula but differ in the nature or sequence of bonding of their atoms or the arrangement of their atoms in space are termed “isomers” and fall within the scope of the invention. Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable minor images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

In the practice of the method of the present invention, an effective amount of any one of the compounds of this invention or a combination of any of the compounds of this invention or a pharmaceutically acceptable salt or ester thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

In detail, the present invention provides for compounds of formula (I):

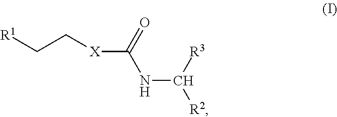

wherein:
X is triazole or imidazole;
R¹ is —NHC(═O)R⁴, —CH₂NHC(═O)R⁴, —NHC(═S)R⁴, —N═C(SCH₃)R⁴ or isoindolyl which is optionally substituted with oxo and/or C₁₋₇ alkoxy;
R² is —C(═O)OH, —C(═O)—C₁₋₇ alkoxy, —(CH₂)₂-phenyl, benzothiophenyl, naphthalenyl, benzodioxolyl, —C(═O)-methoxyphenyl, —CH₂C(═O)-phenyl, —(C(═O))₂NHCH₂-phenyl, phenyl or pyridinyl,
   wherein said pyridinyl is optionally mono-substituted with C₁₋₇ alkyl or C₁₋₇ alkoxy,
   wherein said phenyl is optionally mono- or bi-substituted independently with C₁₋₇ alkyl, —SCH₃, —CF₃, halogen, halo-C₁₋₇ alkyl, C₁₋₇ alkoxy, —OCF₃, —OCH₂-phenyl or cyclopropyl;
R³ is hydrogen, cyano, —CH₂-indolyl or C₁₋₇ alkyl, said C₁₋₇ alkyl optionally substituted with phenyl;
R⁴ is phenyl, pyridinyl, pyridazinyl optionally mono-substituted with C₁₋₇ alkoxy, indenyl optionally mono-substituted with C₁₋₇ alkyl, —(CH₂)ₙ-dihydroindenyl optionally mono-substituted with hydroxy wherein n is 0 or 1, dihydrobenzofuranyl, dihydrobenzodioxinyl, isoindolinyl, benzofuranyl, or benzodioxinyl,
   wherein said phenyl is optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl,
   wherein said pyridinyl is optionally mono- or bi-substituted independently with phenyl, C₁₋₇ alkoxy, —CF₃, —O-chlorophenyl, halogen or C₁₋₇ alkyl;
or a pharmaceutically acceptable salt thereof.

In another embodiment, the present invention provides for compounds of formula (I) wherein X is triazole.

In another embodiment, the present invention provides for compounds of formula (I) wherein X is 1,2,4-triazole.

In another embodiment, the present invention provides for compounds of formula (I) wherein X is imidazole.

In another embodiment, the present invention provides for compounds of formula (I) wherein R¹ is —NHC(═O)R⁴, —CH₂NHC(═O)R⁴, —NHC(═S)R⁴ or —N═C(SCH₃)R⁴.

In another embodiment, the present invention provides for compounds of formula (I) wherein R¹ is —NHC(═O)R⁴, —NHC(═S)R⁴, —N═C(SCH₃)R⁴, oxoisoindolyl or oxoisoindolyl substituted with C₁₋₇ alkoxy.

In another embodiment, the present invention provides for compounds of formula (I) wherein R¹ is —NHC(═O)R⁴.

In another embodiment, the present invention provides for compounds of formula (I) wherein R² is —C(═O)OH, —C(═O)—C₁₋₇ alkoxy, —(CH₂)₂-phenyl, benzothiophenyl, naphthalenyl, benzodioxolyl, —C(═O)-methoxyphenyl, —CH₂C(═O)-phenyl or —(C(═O))₂NHCH₂-phenyl.

In another embodiment, the present invention provides for compounds of formula (I) wherein R² is pyrdinyl, optionally substituted with C₁₋₇ alkyl or C₁₋₇ alkoxy.

In another embodiment, the present invention provides for compounds of formula (I) wherein R² is phenyl, optionally mono- or bi-substituted independently with C₁₋₇ alkyl, —SCH₃, —CF₃, halogen, C₁₋₇ alkoxy, —OCF₃, —OCH₂-phenyl or cyclopropyl.

In a particular embodiment, the present invention provides for compounds of formula (I) wherein R² is —C(═O)—C₁₋₇ alkoxy, phenyl mono-substituted with C₁₋₇ alkyl, or tribromophenyl.

In another embodiment, the present invention provides for compounds of formula (I) wherein R³ is hydrogen.

In another embodiment, the present invention provides for compounds of formula (I) wherein R³ is cyano.

In another embodiment, the present invention provides for compounds of formula (I) wherein R³ is C₁₋₇ alkyl, optionally substituted with phenyl.

In another embodiment, the present invention provides for compounds of formula (I) wherein R³ is —CH₂-indolyl.

In another embodiment, the present invention provides for compounds of formula (I) wherein R⁴ is methoxyoxoisoindolinyl, methylindenyl, oxoisoindolinyl, benzodioxinyl, —CH₂-hydroxyindenyl, benzofuranyl, indenyl, —CH₂-indenyl, phenyl, said phenyl optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl, pyridinyl, said pyridinyl optionally mono- or bi-substituted independently with phenyl, C₁₋₇ alkoxy, —CF₃, —O-chlorophenyl, halogen or C₁₋₇ alkyl, or pyridazinyl, said pyridazinyl optionally substituted with C₁₋₇ alkoxy.

In another embodiment, the present invention provides for compounds of formula (I) wherein R⁴ is methoxyoxoisoindolinyl, methylindenyl, oxoisoindolinyl, benzodioxinyl, —CH₂-hydroxyindenyl, benzofuranyl, indenyl, or —CH₂-indenyl.

In another embodiment, the present invention provides for compounds of formula (I) wherein R⁴ is phenyl, optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl.

In another embodiment, the present invention provides for compounds of formula (I) wherein R⁴ is pyridinyl, optionally mono- or bi-substituted independently with phenyl, C₁₋₇ alkoxy, —CF₃, —O-chlorophenyl, halogen or C₁₋₇ alkyl.

In another embodiment, the present invention provides for compounds of formula (I) wherein R⁴ is pyridazinyl, optionally substituted with C₁₋₇ alkoxy.

In another embodiment, the present invention provides for compounds of formula (I) wherein R⁴ is phenyl, pyridinyl, pyridazinyl optionally mono-substituted with C₁₋₇ alkoxy, indenyl optionally mono-substituted with C₁₋₇ alkyl, —(CH₂)₁₁-dihydroindenyl optionally mono-substituted with hydroxy wherein n is 0 or 1, dihydrobenzofuranyl, or dihydrobenzodioxinyl, wherein said phenyl is optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl, wherein said pyridinyl is optionally mono- or bi-substituted independently with phenyl, C₁₋₇ alkoxy, —CF₃, —O-chlorophenyl, halogen or C₁₋₇ alkyl.

In another embodiment, the present invention provides for compounds of formula (I)
wherein R⁴ is phenyl, pyridinyl, pyridazinyl mono-substituted with methoxy, methylindenyl, —(CH₂)-dihydroindenyl optionally mono-substituted with hydroxy wherein n is 0 or 1, dihydrobenzofuranyl, or dihydrobenzodioxinyl,
wherein said phenyl is optionally mono-, bi- or tri-substituted independently with methoxy, ethoxy, trifluoromethoxy, fluoro, chloro, bromo, —SCH₃, —CF₃, methyl, —SO₂CH₃ or methoxymethyl,
wherein said pyridinyl is optionally mono- or bi-substituted independently with phenyl, methoxy, ethoxy, —CF₃, —O-chlorophenyl, chloro, bromo or methyl.

In a particular embodiment, the present invention provides for compounds of formula (I) wherein R⁴ is trimethoxyphenyl, phenyl substituted with one fluoro and one CF₃, or phenyl substituted with one methoxy and one CF₃.

Another embodiment of the invention relates to a compound of formula (I′):

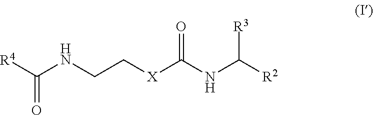

wherein X, R², R³ and R⁴ are as defined above, or a pharmaceutically acceptable salt thereof.

Another embodiment of the invention relates to a compound of formula (I″):

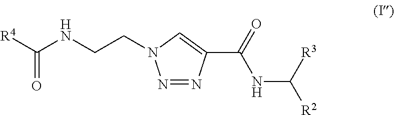

wherein R², R³ and R⁴ are as defined above, or a pharmaceutically acceptable salt thereof.

Another embodiment of the invention relates to a compound of formula (I′″):

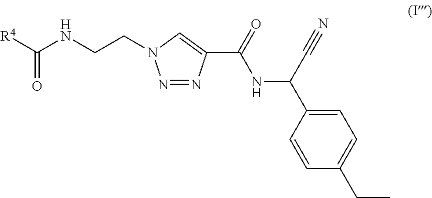

wherein R⁴ is as defined above, particularly R⁴ is trimethoxyphenyl, phenyl substituted with one fluoro and one CF₃, or phenyl substituted with one methoxy and one CF₃, or a pharmaceutically acceptable salt thereof.

In another embodiment, the present invention provides for compounds of formula (I) wherein the compound is:
1-[2-(2,3,4-Trimethoxy-benzoylamino)-ethyl]-1H-[1,2,3]triazole-4-carboxylic acid [cyano-(4-ethyl-phenyl)-methyl]-amide;
1-[2-(2,3,4-Trimethoxy-benzoylamino)-ethyl]-1H-[1,2,3]triazole-4-carboxylic acid [cyano-(4-methylsulfanyl-phenyl)-methyl]-amide;
1-[2-(2,3,4-Trimethoxy-benzoylamino)-ethyl]-1H-[1,2,3]triazole-4-carboxylic acid 4-methyl-benzylamide;
1-[2-(2,3,4-Trimethoxy-benzoylamino)-ethyl]-1H-[1,2,3]triazole-4-carboxylic acid 3,4-dimethyl-benzylamide;
1-[2-(2,3,4-Trimethoxy-benzoylamino)-ethyl]-1H-[1,2,3]triazole-4-carboxylic acid 4-chloro-benzylamide;
1-[2-(2,3,4-Trimethoxy-benzoylamino)-ethyl]-1H-[1,2,3]triazole-4-carboxylic acid 4-methylsulfanyl-benzylamide;
1-[2-(2,3,4-Trimethoxy-benzoylamino)-ethyl]-1H-[1,2,3]triazole-4-carboxylic acid 4-ethyl-benzylamide;
1-[2-(2,3,4-Trimethoxy-benzoylamino)-ethyl]-1H-[1,2,3]triazole-4-carboxylic acid [cyano-(4-methoxy-phenyl)-methyl]-amide;
1-[2-(2-Indan-2-yl-acetylamino)-ethyl]-1H-[1,2,3]triazole-4-carboxylic acid [cyano-(4-ethyl-phenyl)-methyl]-amide;
1-[2-(2,3,4-Trimethoxy-benzoylamino)-ethyl]-1H-[1,2,3]triazole-4-carboxylic acid [cyano-(4-trifluoromethoxy-phenyl)-methyl]-amide;
1-[2-(2-Methoxy-4-trifluoromethoxy-benzoylamino)-ethyl]-1H-[1,2,3]triazole-4-carboxylic acid [cyano-(4-ethyl-phenyl)-methyl]-amide;
1-{2-[(Indane-2-carbonyl)-amino]-ethyl}-1H-[1,2,3]triazole-4-carboxylic acid [cyano-(4-ethyl-phenyl)-methyl]-amide;
1-{2-[(2,3-Dihydro-benzofuran-7-carbonyl)-amino]-ethyl}-1H-[1,2,3]triazole-4-carboxylic acid [cyano-(4-ethyl-phenyl)-methyl]-amide;
1-[2-(2,5-Dichloro-benzoylamino)-ethyl]-1H-[1,2,3]triazole-4-carboxylic acid [cyano-(4-ethyl-phenyl)-methyl]-amide;
6-Phenyl-pyridine-2-carboxylic acid [2-(4-{[cyano-(4-ethyl-phenyl)-methyl]-carbamoyl}-[1,2,3]triazol-1-yl)-ethyl]amide;
1-[2-(2,3-Dimethoxy-benzoylamino)-ethyl]-1H-[1,2,3]triazole-4-carboxylic acid [cyano-(4-ethyl-phenyl)-methyl]-amide;
3-Methoxy-pyridine-2-carboxylic acid [2-(4-{[cyano-(4-ethyl-phenyl)-methyl]-carbamoyl}-[1,2,3]triazol-1-yl)-ethyl]-amide;
1-{2-[(2,3-Dihydro-benzo[1,4]dioxine-5-carbonyl)-amino}-ethyl}-1H-[1,2,3]triazole-4-carboxylic acid [cyano-(4-ethyl-phenyl)-methyl]-amide;
1-[2-(2-Methoxy-4-methylsulfanyl-benzoylamino)-ethyl]-1H-[1,2,3]triazole-4-carboxylic acid [cyano-(4-ethyl-phenyl)-methyl]-amide;
1-[2-(4-Chloro-2-methoxy-benzoylamino)-ethyl]-1H-[1,2,3]triazole-4-carboxylic acid [cyano-(4-ethyl-phenyl)-methyl]-amide;
4-Chloro-6-methyl-pyridine-2-carboxylic acid [2-(4-{[cyano-(4-ethyl-phenyl)-methyl]-carbamoyl}-[1,2,3]triazol-1-yl)-ethyl]-amide;
1-{2-[2-(2-Hydroxy-indan-2-yl)-acetylamino]-ethyl}-1H-[1,2,3]triazole-4-carboxylic acid [cyano-(4-ethyl-phenyl)-methyl]-amide;
1-[2-(2-Methoxy-4-trifluoromethyl-benzoylamino)-ethyl]-1H-[1,2,3]triazole-4-carboxylic acid [cyano-(4-ethyl-phenyl)-methyl]-amide;
1-[2-(2-Trifluoromethoxy-benzoylamino)-ethyl]-1H-[1,2,3]triazole-4-carboxylic acid [cyano-(4-ethyl-phenyl)-methyl]-amide;
1-{2-[(3-Methyl-1H-indene-2-carbonyl)-amino]-ethyl}-1H-[1,2,3]triazole-4-carboxylic acid [cyano-(4-ethyl-phenyl)-methyl]-amide;
6-Methoxy-pyridine-2-carboxylic acid [2-(4-{[cyano-(4-ethyl-phenyl)-methyl]-carbamoyl}-[1,2,3]triazol-1-yl)-ethyl]-amide;
4-Chloro-pyridine-2-carboxylic acid [2-(4-{[cyano-(4-ethyl-phenyl)-methyl]-carbamoyl}-[1,2,3]triazol-1-yl)-ethyl]-amide;
5-Ethoxy-pyridine-2-carboxylic acid [2-(4-{[cyano-(4-ethyl-phenyl)-methyl]-carbamoyl}-[1,2,3]triazol-1-yl)-ethyl]-amide;
N-[2-(4-{[Cyano-(4-ethyl-phenyl)-methyl]-carbamoyl}-[1,2,3]triazol-1-yl)-ethyl]-2-methoxy-nicotinamide;
5-Bromo-N-[2-(4-{[cyano-(4-ethyl-phenyl)-methyl]-carbamoyl}-[1,2,3]triazol-1-yl)-ethyl]-4-methoxy-nicotinamide;
1-[2-(4-Chloro-2-fluoro-benzoylamino)-ethyl]-1H-[1,2,3]triazole-4-carboxylic acid [cyano-(4-ethyl-phenyl)-methyl]-amide;
1-[2-(2-Fluoro-5-methoxy-benzoylamino)-ethyl]-1H-[1,2,3]triazole-4-carboxylic acid [cyano-(4-ethyl-phenyl)-methyl]-amide;
1-[2-(2-Methoxymethyl-benzoylamino)-ethyl]-1H-[1,2,3]triazole-4-carboxylic acid [cyano-(4-ethyl-phenyl)-methyl]-amide;
N-[2-(4-{[Cyano-(4-ethyl-phenyl)-methyl]-carbamoyl}-[1,2,3]triazol-1-yl)-ethyl]-2-methoxy-6-methyl-nicotinamide;
N-[2-(4-{[Cyano-(4-ethyl-phenyl)-methyl]-carbamoyl}-[1,2,3]triazol-1-yl)-ethyl]-2,6-dimethoxy-nicotinamide;
1-[2-(4-Fluoro-2-methoxy-benzoylamino)-ethyl]-1H-[1,2,3]triazole-4-carboxylic acid [cyano-(4-ethyl-phenyl)-methyl]-amide;
1-[2-(2-Fluoro-5-trifluoromethyl-benzoylamino)-ethyl]-1H-[1,2,3]triazole-4-carboxylic acid [cyano-(4-ethyl-phenyl)-methyl]-amide;
1-[2-(2-Fluoro-4-trifluoromethyl-benzoylamino)-ethyl]-1H-[1,2,3]triazole-4-carboxylic acid [cyano-(4-ethyl-phenyl)-methyl]-amide;
1-[2-(2-Methoxy-5-trifluoromethyl-benzoylamino)-ethyl]-1H-[1,2,3]triazole-4-carboxylic acid [cyano-(4-ethyl-phenyl)-methyl]-amide;
6-Methoxy-pyridazine-3-carboxylic acid [2-(4-{[cyano-(4-ethyl-phenyl)-methyl]-carbamoyl}-[1,2,3]triazol-1-yl)-ethyl]-amide;
1-[2-(2-Chloro-4-methanesulfonyl-benzoylamino)-ethyl]-1H-[1,2,3]triazole-4-carboxylic acid [cyano-(4-ethyl-phenyl)-methyl]-amide;
1-[2-(5-Methanesulfonyl-2-methoxy-benzoylamino)-ethyl]-1H-[1,2,3]triazole-4-carboxylic acid [cyano-(4-ethyl-phenyl)-methyl]-amide;
5-(4-Chloro-phenoxy)-pyridine-2-carboxylic acid [2-(4-{[cyano-(4-ethyl-phenyl)-methyl]-carbamoyl}-[1,2,3]triazol-1-yl)-ethyl]-amide;
5-Chloro-pyridine-2-carboxylic acid [2-(4-{[cyano-(4-ethyl-phenyl)-methyl]-carbamoyl}-[1,2,3]triazol-1-yl)-ethyl]-amide;
5-Trifluoromethyl-pyridine-2-carboxylic acid [2-(4-{[cyano-(4-ethyl-phenyl)-methyl]-carbamoyl}-[1,2,3]triazol-1-yl)-ethyl]-amide;
1-[2-(1-Oxo-1,3-dihydro-isoindol-2-yl)-ethyl]-1H-[1,2,3]triazole-4-carboxylic acid [cyano-(4-ethyl-phenyl)-methyl]-amide;
1-[2-(5-Methoxy-1-oxo-1,3-dihydro-isoindol-2-yl)-ethyl]-1H-[1,2,3]triazole-4-carboxylic acid [cyano-(4-ethyl-phenyl)-methyl]-amide;
1-[2-(2,3,4-Trimethoxy-benzoylamino)-ethyl]-1H-[1,2,3]triazole-4-carboxylic acid (benzo[b]thiophen-5-yl-cyano-methyl)-amide;
1-[2-(2,3,4-Trimethoxy-benzoylamino)-ethyl]-1H-[1,2,3]triazole-4-carboxylic acid (cyano-naphthalen-2-yl-methyl)-amide;
1-[2-(2,3,4-Trimethoxy-benzoylamino)-ethyl]-1H-[1,2,3]triazole-4-carboxylic acid [(R)-cyano-(4-ethyl-phenyl)-methyl]-amide;
1-[2-(2,3,4-Trimethoxy-benzoylamino)-ethyl]-1H-[1,2,3]triazole-4-carboxylic acid [(S)-cyano-(4-ethyl-phenyl)-methyl]-amide;
1-[2-(2,3,4-Trimethoxy-benzoylamino)-ethyl]-1H-[1,2,3]triazole-4-carboxylic acid [cyano-(3-methoxy-phenyl)-methyl]-amide;
1-[2-(2,3,4-Trimethoxy-benzoylamino)-ethyl]-1H-[1,2,3]triazole-4-carboxylic acid (benzo[1,3]dioxol-5-yl-cyano-methyl)-amide;
1-[2-(2,3,4-Trimethoxy-benzoylamino)-ethyl]-1H-[1,2,3]triazole-4-carboxylic acid [(4-bromo-phenyl)-cyano-methyl]-amide;
1-[2-(2,3,4-Trimethoxy-benzoylamino)-ethyl]-1H-[1,2,3]triazole-4-carboxylic acid [cyano-(3-methylsulfanyl-phenyl)-methyl]-amide;
1-[2-(2,3,4-Trimethoxy-benzoylamino)-ethyl]-1H-[1,2,3]triazole-4-carboxylic acid [(4-benzyloxy-phenyl)-cyano-methyl]-amide;
1-[2-(2,3,4-Trimethoxy-benzoylamino)-ethyl]-1H-[1,2,3]triazole-4-carboxylic acid (cyano-naphthalen-1-yl-methyl)-amide;
1-[2-(2,3,4-Trimethoxy-benzoylamino)-ethyl]-1H-[1,2,3]triazole-4-carboxylic acid [cyano-(4-cyclopropyl-phenyl)-methyl]-amide;
1-[2-(2,3,4-Trimethoxy-benzoylamino)-ethyl]-1H-[1,2,3]triazole-4-carboxylic acid [(2-bromo-phenyl)-cyano-methyl]-amide;
1-[2-(2,3,4-Trimethoxy-benzoylamino)-ethyl]-1H-[1,2,3]triazole-4-carboxylic acid [cyano-(4-trifluoromethyl-phenyl)-methyl]-amide;
1-[2-(2,3,4-Trimethoxy-benzoylamino)-ethyl]-1H-[1,2,3]triazole-4-carboxylic acid [cyano-(4-isopropyl-phenyl)-methyl]-amide;
1-[2-(2,3,4-Trimethoxy-benzoylamino)-ethyl]-1H-[1,2,3]triazole-4-carboxylic acid (cyano-p-tolyl-methyl)-amide;
1-[2-(2,3,4-Trimethoxy-benzoylamino)-ethyl]-1H-[1,2,3]triazole-4-carboxylic acid [cyano-(5-ethyl-pyridin-2-yl)-methyl]-amide;
1-[2-(2,3,4-Trimethoxy-benzoylamino)-ethyl]-1H-[1,2,3]triazole-4-carboxylic acid [cyano-(4-propyl-phenyl)-methyl]-amide;
1-[2-(2,3,4-Trimethoxy-benzoylamino)-ethyl]-1H-[1,2,3]triazole-4-carboxylic acid [cyano-(6-methoxy-pyridin-3-yl)-methyl]-amide;
1-[2-(2,3,4-Trimethoxy-benzoylamino)-ethyl]-1H-[1,2,3]triazole-4-carboxylic acid (benzo[b]thiophen-6-yl-cyano-methyl)-amide;
1-[2-(2,3,4-Trimethoxy-benzoylamino)-ethyl]-1H-[1,2,3]triazole-4-carboxylic acid [cyano-(4-isopropoxy-phenyl)-methyl]-amide;
1-[2-(2,3,4-Trimethoxy-benzoylamino)-ethyl]-1H-[1,2,3]triazole-4-carboxylic acid [(4-chloro-phenyl)-cyano-methyl]-amide;
1-[2-(2,5-Dichloro-benzoylamino)-ethyl]-1H-[1,2,3]triazole-4-carboxylic acid [cyano-(4-trifluoromethyl-phenyl)-methyl]-amide;
1-{2-[(3-Methyl-1H-indene-2-carbonyl)-amino]-ethyl}-1H-[1,2,3]triazole-4-carboxylic acid [cyano-(4-trifluoromethyl-phenyl)-methyl]-amide;
1-[2-(2-Methoxy-4-trifluoromethoxy-benzoylamino)-ethyl]-1H-[1,2,3]triazole-4-carboxylic acid [cyano-(4-trifluoromethyl-phenyl)-methyl]-amide;
1-[2-(2-Methoxy-4-trifluoromethyl-benzoylamino)-ethyl]-1H-[1,2,3]triazole-4-carboxylic acid [cyano-(4-trifluoromethyl-phenyl)-methyl]-amide;
1-[2-(2-Fluoro-4-trifluoromethyl-benzoylamino)-ethyl]-1H-[1,2,3]triazole-4-carboxylic acid [cyano-(4-trifluoromethyl-phenyl)-methyl]-amide;
1-[2-(2-Fluoro-4-methoxy-benzoylamino)-ethyl]-1H-[1,2,3]triazole-4-carboxylic acid [cyano-(4-trifluoromethyl-phenyl)-methyl]-amide;
1-[2-(2-Fluoro-4-methyl-benzoylamino)-ethyl]-1H-[1,2,3]triazole-4-carboxylic acid [cyano-(4-trifluoromethyl-phenyl)-methyl]-amide;
1-[2-(2-Fluoro-4-trifluoromethyl-benzoylamino)-ethyl]-1H-[1,2,3]triazole-4-carboxylic acid [cyano-(4-cyclopropyl-phenyl)-methyl]-amide;
1-[2-(2,3,4-Trimethoxy-benzoylamino)-ethyl]-1H-[1,2,3]triazole-4-carboxylic acid [2-(4-methoxy-phenyl)-2-oxo-ethyl]-amide;
1-[2-(2-Fluoro-4-trifluoromethyl-benzoylamino)-ethyl]-1H-[1,2,3]triazole-4-carboxylic acid (3-oxo-3-phenyl-propyl)-amide;
1-[2-(2-Fluoro-4-trifluoromethyl-benzoylamino)-ethyl]-1H-[1,2,3]triazole-4-carboxylic acid (1-methyl-3-phenyl-propyl)-amide;
(S)-4-Phenyl-2-({1-[2-(2,3,4-trimethoxy-benzoylamino)-ethyl]-1H-[1,2,3]triazole-4-carbonyl}-amino)-butyric acid ethyl ester;
(S)-2-({1-[2-(2-Fluoro-4-trifluoromethyl-benzoylamino)-ethyl]-1H-[1,2,3]triazole-4-carbonyl}-amino)-4-phenyl-butyric acid ethyl ester;
(S)-2-({1-[2-(2-Fluoro-4-trifluoromethyl-benzoylamino)-ethyl]-1H-[1,2,3]triazole-4-carbonyl}-amino)-4-phenyl-butyric acid;
(S)-4-Phenyl-2-({1-[2-(2,3,4-trimethoxy-benzoylamino)-ethyl]-1H-[1,2,3]triazole-4-carbonyl}-amino)-butyric acid;
(S)-2-({1-[2-(2-Fluoro-4-trifluoromethyl-benzoylamino)-ethyl]-1H-[1,2,3]triazole-4-carbonyl}-amino)-3-(1H-indol-2-yl)-propionic acid methyl ester;
1-[3-(2-Methoxy-4-trifluoromethyl-benzoylamino)-propyl]-1H-[1,2,3]triazole-4-carboxylic acid [cyano-(4-ethyl-phenyl)-methyl]-amide;
1-[2-(2,3,4-Trimethoxy-thiobenzoylamino)-ethyl]-1H-[1,2,3]triazole-4-carboxylic acid [cyano-(4-ethyl-phenyl)-methyl]-amide;
N-[2-(4-{[Cyano-(4-ethyl-phenyl)-methyl]-carbamoyl}-[1,2,3]triazol-1-yl)-ethyl]-2,3,4-trimethoxy-thiobenzimidic acid methyl ester;
1-[2-(2,3,4-Trimethoxy-benzoylamino)-ethyl]-1H-[1,2,3]triazole-4-carboxylic acid ((S)-1-benzyl-2-benzylcarbamoyl-2-oxo-ethyl)-amide;
5-[2-(2,3,4-Trimethoxy-benzoylamino)-ethyl]-2H-[1,2,4]triazole-3-carboxylic acid [cyano-(4-ethyl-phenyl)-methyl]-amide;
5-[2-(2-Methoxy-4-trifluoromethyl-benzoylamino)-ethyl]-2H-[1,2,4]triazole-3-carboxylic acid [cyano-(4-ethyl-phenyl)-methyl]-amide;
5-[2-(2-Fluoro-4-trifluoromethyl-benzoylamino)-ethyl]-2H-[1,2,4]triazole-3-carboxylic acid [cyano-(4-ethyl-phenyl)-methyl]-amide;
1-[2-(2-Methoxy-4-trifluoromethyl-benzoylamino)-ethyl]-1H-imidazole-4-carboxylic acid [cyano-(4-ethyl-phenyl)-methyl]-amide;
1-[2-(2-Methoxy-4-trifluoromethyl-benzoylamino)-ethyl]-1H-imidazole-4-carboxylic acid [(4-bromo-phenyl)-cyano-methyl]-amide; or
1-[2-(2-Fluoro-4-trifluoromethyl-benzoylamino)-ethyl]-1H-imidazole-4-carboxylic acid [cyano-(4-ethyl-phenyl)-methyl]-amide; or
pharmaceutically acceptable salts thereof.

In another embodiment, the present invention provides for compounds of formula (I) wherein the compound is:
N-[cyano-(4-ethylphenyl)methyl]-1-[2-[(2,3,4-trimethoxybenzoyl)amino]ethyl]triazole-4-carboxamide;
N-[(4-ethylphenyl)methyl]-1-[2-[(2,3,4-trimethoxybenzoyl)amino]ethyl]triazole-4-carboxamide;
N-[cyano-(4-ethylphenyl)methyl]-1-[2-[[2-methoxy-4-(trifluoromethyl)benzoyl]amino]ethyl]triazole-4-carboxamide;
N-[cyano-(4-ethylphenyl)methyl]-1-[2-[[2-fluoro-4-(trifluoromethyl)benzoyl]amino]ethyl]triazole-4-carboxamide;
N-[cyano-(4-propylphenyl)methyl]-1-[2-[(2,3,4-trimethoxybenzoyl)amino]ethyl]triazole-4-carboxamide;
ethyl (2S)-4-phenyl-2-[[1-[2-[(2,3,4-trimethoxybenzoyl)amino]ethyl]triazole-4-carbonyl]amino]butanoate;
methyl (2S)-2-[[1-[2-[[2-fluoro-4-(trifluoromethyl)benzoyl]amino]ethyl]triazole-4-carbonyl]amino]-3-(1H-indol-2-yl)propanoate;
N-[cyano-(4-ethylphenyl)methyl]-3-[2-[(2,3,4-trimethoxybenzoyl)amino]ethyl]-1H-1,2,4-triazole-5-carboxamide;
N-[cyano-(4-ethylphenyl)methyl]-3-[2-[[2-methoxy-4-(trifluoromethyl)benzoyl]amino]ethyl]-1H-1,2,4-triazole-5-carboxamide;
N-[cyano-(4-ethylphenyl)methyl]-3-[2-[[2-fluoro-4-(trifluoromethyl)benzoyl]amino]ethyl]-1H-1,2,4-triazole-5-carboxamide; or
N-[(4-bromophenyl)-cyanomethyl]-1-[2-[[2-methoxy-4-(trifluoromethyl)benzoyl]amino]ethyl]imidazole-4-carboxamide; or
pharmaceutically acceptable salts thereof.

In another embodiment, the invention provides for a pharmaceutical composition, comprising a therapeutically effective amount of a compound according to formula (I) and a pharmaceutically acceptable carrier.

In another embodiment, the invention provides for a compound according to formula (I) for use as a therapeutically active substance.

In another embodiment, the invention provides for the use of a compound according to formula (I) for the treatment or prophylaxis of an inflammatory disease or disorder selected from rheumatoid arthritis, lupus and irritable bowel disease.

In another embodiment, the invention provides for the use of a compound according to formula (I) for the preparation of a medicament for the treatment or prophylaxis of an inflammatory disease or disorder selected from rheumatoid arthritis, lupus and irritable bowel disease.

In another embodiment, the invention provides for a compound according to formula (I) for the treatment or prophylaxis of an inflammatory disease or disorder selected from rheumatoid arthritis, lupus and irritable bowel disease.

In another embodiment, the invention provides for a method for treating an inflammatory disease or disorder selected from rheumatoid arthritis, lupus and irritable bowel disease (IBD), comprising the step of administering a therapeutically effective amount of a compound according to formula (I) to a subject in need thereof.

In another embodiment, provided is an invention as hereinbefore described.

### Synthesis

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Compounds of the invention may be made by any number of conventional means. For example, they may be made according to the processes outlined in the Schemes below.

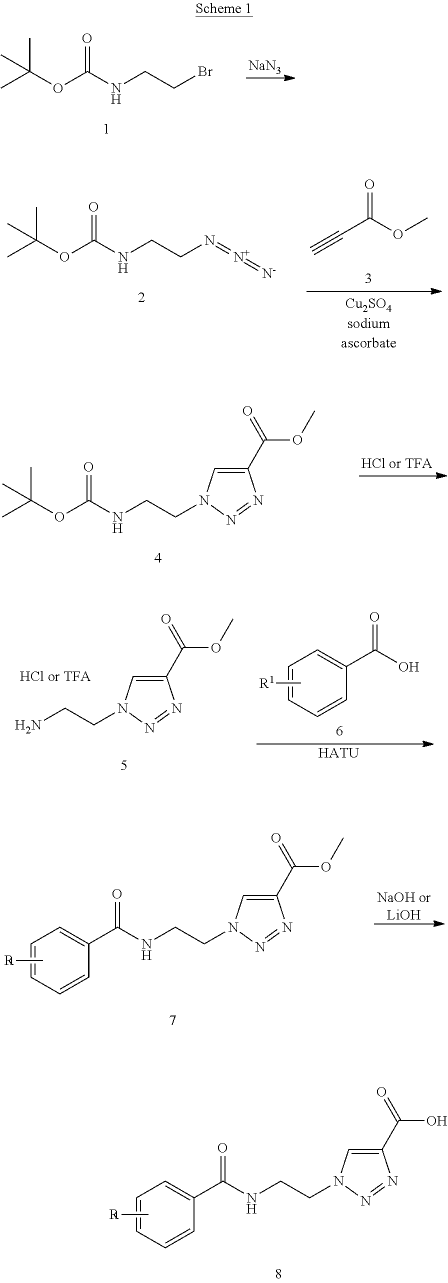

The bromide 1 can be converted to azide 2 using sodium azide then reacted with methyl propiolate 3 in the presence of copper (II) sulfate and sodium ascorbate to afford 1,2,3-triazole 4 in a regioselective manner. The N-Boc protecting group can be removed using a strong acid such as HCl or TFA. The resultant amine salt 5 can be coupled with variably substituted acids 6 using an activating reagent such as HATU to provide ester 7. Hydrolysis under basic conditions gave acid 8.

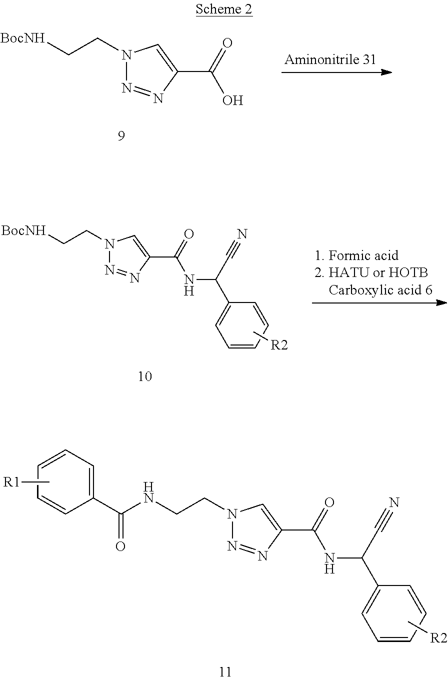

After activation, 1-(2-(tert-butoxycarbonylamino)ethyl)-1H-1,2,3-triazole-4-carboxylic acid 9 (commercial: Zerenex, product number: ZXH001061) can be reacted with a suitable aminonitrile (31) to afford compound 10. Cleavage of the Boc protecting group using preferentially formic acid afforded the corresponding amine which can be further reacted with the acid 6 leading to compound 11.

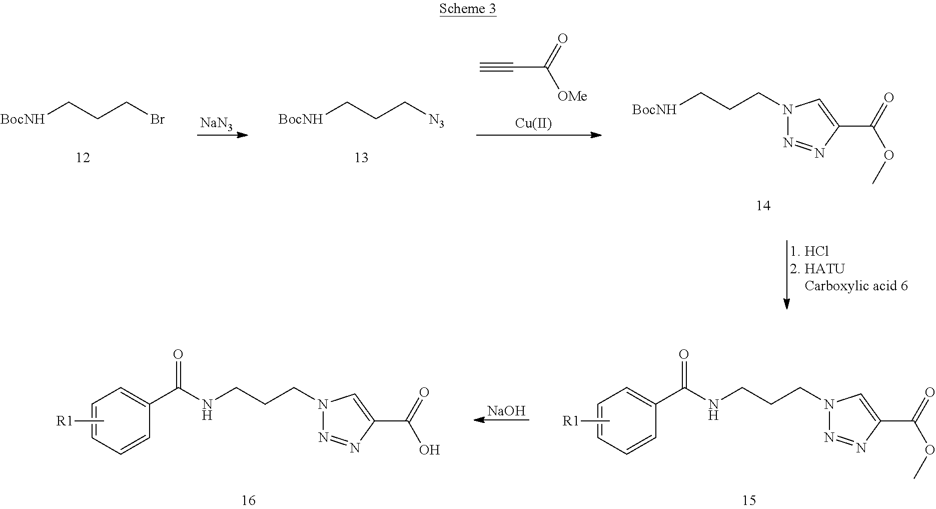

The azide 13 produced by bromine displacement on 12 can be reacted with propynoic acid methyl ester using copper-catalysed Click annelation. The amine function of the 1,2,3-triazole 14 can be deprotected and reacted with the corresponding carboxylic acid after activation. Saponification of the ester in basic conditions afforded the carboxylic acid 16.

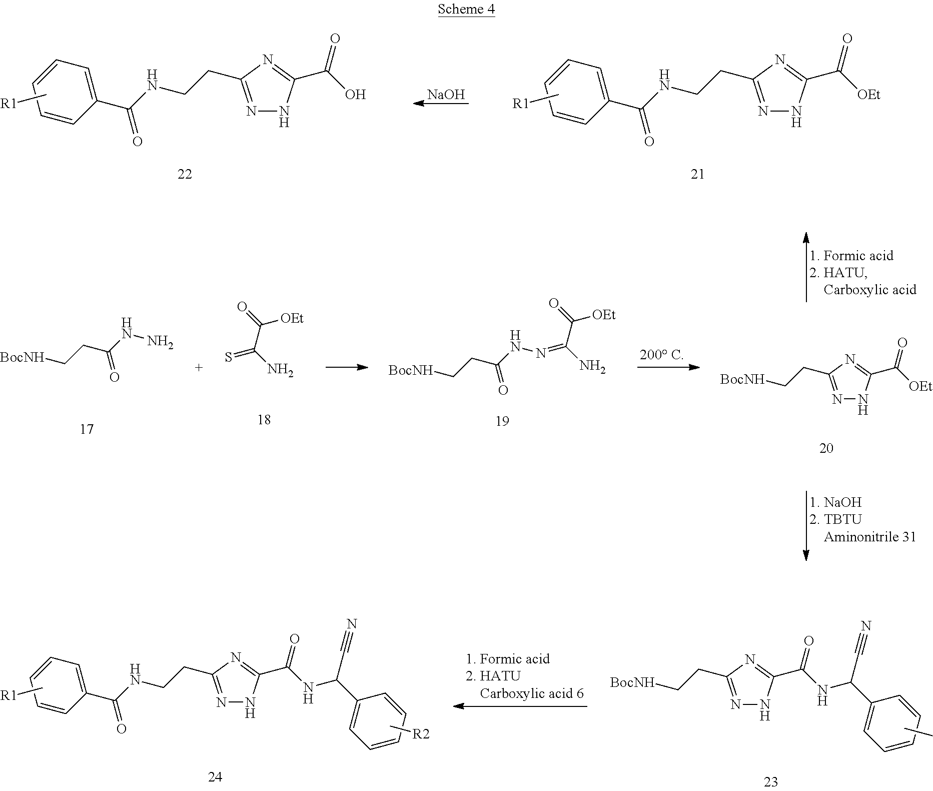

Tert-butyl 3-hydrazinyl-3-oxopropylcarbamate 17 and ethyl 2-amino-2-thioxoacetate 18 can be reacted together in neat conditions. The resulting ethyl 2-amino-2-(2-(3-(tert-butoxycarbonylamino)propanoyl)hydrazono)acetate 19 can be cyclized to 1,2,4-triazole 20 at high temperature. 2 routes can be then pursued. The Boc of the triazole can be first cleaved and the resulting amine reacted with the appropriate acid. Then the ester can be hydrolysed in basic conditions to afford the carboxylic acid 22. Alternatively, the ester 20 can be hydrolyzed first in basic condition and the resulting carboxylate can be reacted with an aminonitrile 31 after activation. Cleavage of the Boc protecting group using preferentially trifluoroacetic acid afforded the corresponding amine which can be further reacted with an appropriate acid to afford compound 24.

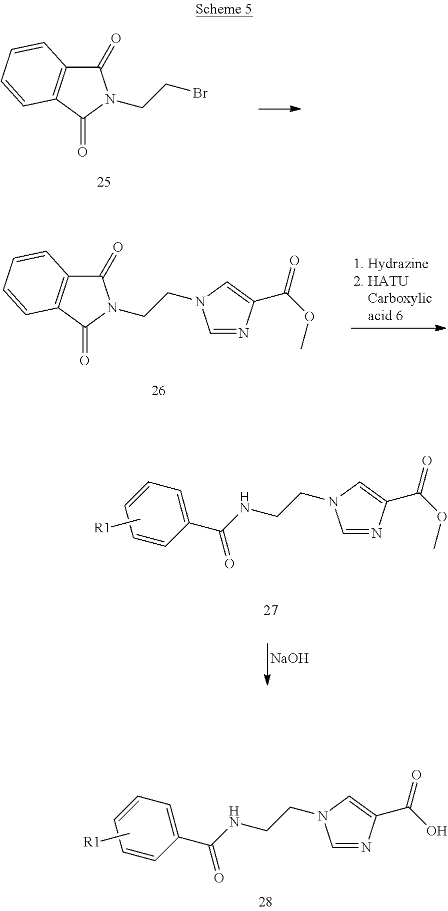

The bromine in compound 25 can be displaced by the imidazole. The dioxoisoindoline protecting group can be cleaved using hydrazine and the resulting free amine can be reacted with the appropriate activated carboxylic acid to afford compound 27. The ester can be hydrolysed in basic conditions leading to the carboxylate 28

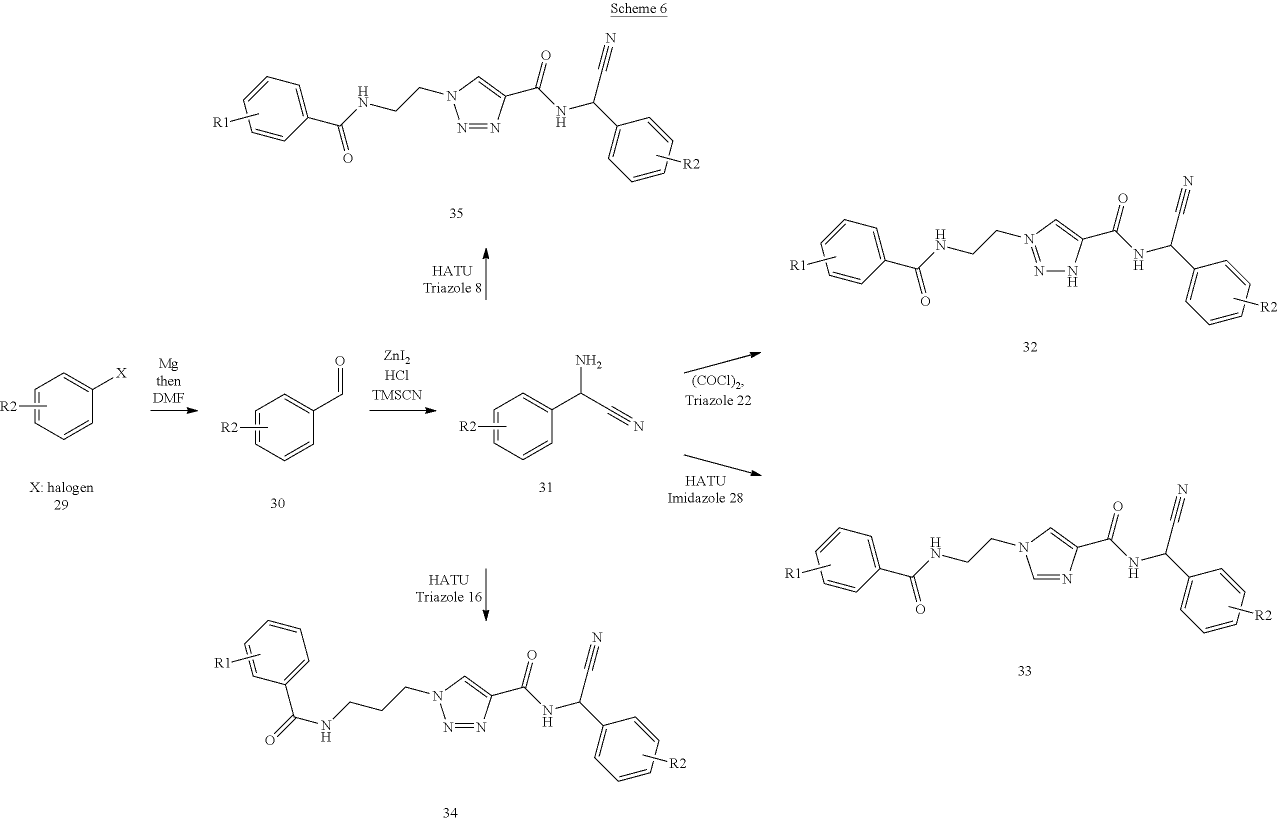

In the cases where aminonitrile 31 can be not commercially available, it could be conveniently prepared by a modified Strecker reaction. Optionally, when the corresponding aldehyde 30 can be not commercially available, it could conveniently be prepared for example by metallation of the corresponding halogens 29 with magnesium and subsequent reaction with N,N-dimethylformamide. The resulting aminonitrile 31 can be coupled to various triazoles 8, 16, 22 or imidazole 28 carboxylic acids.

### EXAMPLES

Although certain exemplary embodiments are depicted and described herein, the compounds of the present invention can be prepared using appropriate starting materials according to the methods described generally herein and/or by methods available to one of ordinary skill in the art. All reactions involving air-sensitive reagents were performed under an inert atmosphere. Reagents were used as received from commercial suppliers unless otherwise noted.

### Intermediate 1

### 1-(2-Amino-ethyl)-1H-[1,2,3]triazole-4-carboxylic acid methyl ester hydrochloride

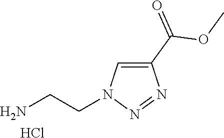

Step 1

2-(Boc-amino)ethyl bromide (5.0 g, 22.3 mmol) was dissolved in 50 ml DMF and sodium azide (1.6 g, 24.5 mmol) was added. The reaction mixture was stirred at 80° C. for 12 h. The reaction mixture was diluted with diethyl ether (200 ml) and washed with water (3×) and brine (2×). The organic phase was dried over sodium sulfate and concentrated under reduced pressure to afford 3.9 g (94%) (2-azido-ethyl)-carbamic acid tert-butyl ester as a colorless viscous oil. GC/MS: (M+H)⁺=187.191.

Step 2

(2-Azido-ethyl)-carbamic acid tert-butyl ester (3.9 g, 20.8 mmol) and methyl propiolate (3.5 g, 3.71 ml, 41.7 mmol) were dissolved in 50 ml tert-butanol. A 1.0 M aq. solution of copper(II) sulfate pentahydrate (4.17 ml, 4.17 mmol) was added followed by a 1.0 M aq. solution of sodium ascorbate (16.7 ml, 16.7 mmol). The reaction mixture was stirred at room temperature for 60 h. The reaction mixture was quenched with 150 ml water and extracted with EtOAc (3×80 ml). The organic layers were combined, dried over sodium sulfate, filtered and concentrated. The residue was chromatographed over 70 g silica gel with EtOAc/dichloromethane (gradient: 0-40% EtOAc). All fractions containing product were combined and concentrated to afford 3.2 g (57%) 1-(2-tert-butoxycarbonylamino-ethyl)-1H-[1,2,3]triazole-4-carboxylic acid methyl ester as an off-white solid. LC/HR-MS: (M+H)⁺=271.1401.

Step 3

1-(2-tert-Butoxycarbonylamino-ethyl)-1H-[1,2,3]triazole-4-carboxylic acid methyl ester (1.75 g, 6.47 mmol) was dissolved in 4N HCl in dioxane (16.2 ml, 64.7 mmol) and stirred at room temperature for 3 h. The solvent was evaporated to afford 1.32 g (99%) 1-(2-amino-ethyl)-1H-[1,2,3]triazole-4-carboxylic acid methyl ester hydrochloride as a white solid. LC/HR-MS: (M+H)⁺=171.0883

### Intermediate 2

### 1-[2-(2,3,4-Trimethoxy-benzoylamino)-ethyl]-1H-[1,2,3]triazole-4-carboxylic acid

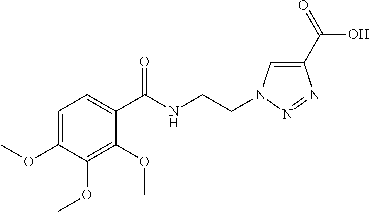 [SMILES: COc1ccc(C(=O)NCCn2cc(C(=O)O)nn2)c(OC)c1OC]

Step 1

A flask was charged with 2,3,4-trimethoxybenzoic acid (1.29 g, 6.08 mmol), 57 ml N,N-dimethylacetamide and N,N-diisopropylethylamine (2.9 ml, 16.9 mmol). The reaction mixture was cooled to 0° C. HATU (2.83 g, 7.45 mmol) was added and the reaction mixture was stirred at 0° C. for 1 h. 1-(2-Amino-ethyl)-1H-[1,2,3]triazole-4-carboxylic acid methyl ester hydrochloride (cf. Intermediate 1, step 3, 1.4 g, 6.78 mmol) was added and the reaction mixture was stirred at room temperature overnight. The reaction mixture was quenched with 1.0 M HCl and extracted with EtOAc. The organic layer was washed with aqueous KHCO₃, water and brine then concentrated and dried under high vacuum. The residue was triturated with diethyl ether to afford 1-[2-(2,3,4-trimethoxy-benzoylamino)-ethyl]-1H-[1,2,3]triazole-4-carboxylic acid methyl ester as a light brown semisolid which was used without further purification.

Step 2

A flask was charged with 1-[2-(2,3,4-trimethoxy-benzoylamino)-ethyl]-1H-[1,2,3]triazole-4-carboxylic acid methyl ester (2.22 g, 6.1 mmol) and 60 ml methanol. Then, 1.0 M NaOH (24 ml, 24 mmol) was added and the reaction mixture was stirred at room temperature overnight. The reaction mixture was partially concentrated then taken up in water, acidified with 1.0 M HCl and extracted twice with 200 ml EtOAc. The organic layers were combined, dried over sodium sulfate, filtered and concentrated. The residue was taken up in 70 ml dichloromethane, 40 ml EtOAc and 10 ml methanol and then concentrated to a volume of ˜30 ml. Diethyl ether was added and the suspension was filtered, rinsed with diethyl ether and dried under high vacuum to afford 1.8 g (84%) 1-[2-(2,3,4-trimethoxy-benzoylamino)-ethyl]-1H-[1,2,3]triazole-4-carboxylic acid as a white solid. LC/HR-MS: (M+H)⁺=351.1293.

### Intermediate 3

### 1-(2-Amino-ethyl)-1H-[1,2,3]triazole-4-carboxylic acid [cyano-(4-ethyl-phenyl)-methyl]-amide

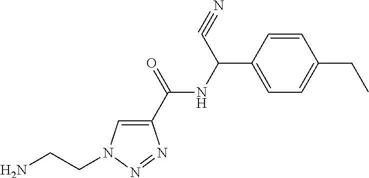 [SMILES: CCc1ccc(C(C#N)NC(=O)c2cn(CCN)nn2)cc1]

Step 1

4-Ethylbenzaldehyde (7 g, 7.15 ml, 52.2 mmol) and trimethylsilanecarbonitrile (9.81 g, 12.4 ml, 93.9 mmol) were combined under N₂ atmosphere. The reaction was cooled to 0° C. and zinc iodide (42.5 mg, 130 μmol) was added in 2 portions. The reaction mixture was stirred at room temperature for 15 min. The reaction mixture was then transferred into an autoclave and ammonia in methanol (7M, 224 ml, 1.57 mol), was added. The reaction was stirred at 40° C. for 2 h (oil bath=50° C.). The solvent was evaporated and the residue suspended in diethylether and filtered. A solution of hydrochloric acid in dioxane (4M) was added. The precipitate was filtered, washed with diethyl ether and dried in vacuo to afford 4.5 g (44%) of 2-amino-2-(4-ethylphenyl)acetonitrile hydrochloride as a light brown solid. MS+(m/z): 161.4 (M+H)⁺.

Step 2

In a 10 mL round-bottomed flask, 1-(2-(tert-butoxycarbonylamino)ethyl)-1H-1,2,3-triazole-4-carboxylic acid (300 mg, 1.17 mmol ,commercial from Zerenex), 2-(1H-7-azabenzotriazol-1-yl)-1,1,3,3-tetramethyl uronium hexafluorophosphate methanaminium (HATU) (668 mg, 1.76 mmol) and diisopropylethylamine (605 mg, 818 μl, 4.68 mmol) were combined in dimethylacetamide (5.00 ml) at 0° C. The reaction mixture was stirred at 0° C. for 1 h, then 2-amino-2-(4-ethylphenyl)acetonitrile hydrochloride (230 mg, 1.17 mmol) was added. The reaction mixture was allowed to warm up to room temperature and stirred for 16 h.

The crude reaction mixture was concentrated in vacuo and the residue was partitioned between dichloromethane and a saturated aqueous solution of sodium hydrogenocarbonate. The aqueous layer was extracted with dichloromethane and the combined organic layers were washed with water, brine and dried in vacuo. The residue was purified by chromatography over silica gel (20 g, heptane/dichloromethane/methanol 1:1:1 to 0:98:2). One fraction was isolated and dried in vacuo, affording 417 mg (89%) of [2-(4-{[cyano-(4-ethyl-phenyl)-methyl]-carbamoyl}-[1,2,3]triazol-1-yl)-ethyl]-carbamic acid tert-butyl ester as a brown solid. LC/HR-MS: (M−H)⁻=397.2001.

Step 3

In a 25 mL round-bottomed flask, [2-(4-{[cyano-(4-ethyl-phenyl)-methyl)carbamoyl}-[1,2,3]triazol-1-yl)-ethyl]-carbamic acid tert-butyl ester (400 mg, 1.00 mmol) was combined with formic acid (4.62 g, 3.85 ml, 100 mmol). The reaction mixture was stirred at room temperature for 2 h. The crude reaction mixture was concentrated in vacuo and the residue was partitioned between dichloromethane and 5% aqueous solution of sodium carbonate to adjust the pH to 10-12. The aqueous layer was extracted with dichloromethane and the combined organic layer was dried in vacuo to afford 273 mg (91%) of 1-(2-amino-ethyl)-1H-[1,2,3]triazole-4-carboxylic acid [cyano-(4-ethyl-phenyl)-methyl]-amide as a brown solid. This solid was used without further purification. LC/HR-MS: (M+H)⁺=299.1617.

Intermediate 4

### 1-(2-Amino-ethyl)-1H-[1,2,3]triazole-4-carboxylic acid [cyano-(4-trifluoromethyl-phenyl)-methyl]-amide

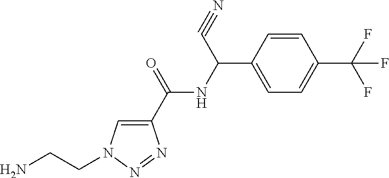 [SMILES: N#CC(NC(=O)c1cn(CCN)nn1)c1ccc(C(F)(F)F)cc1]

Step 1

Amino-(4-trifluoromethyl-phenyl)-acetonitrile hydrochloride was prepared in analogy to step 1 of the synthesis of Intermediate 3 from 4-trifluoromethyl-benzaldehyde. Off-white solid. GC-EI-MS: (M)⁺=200.

Step 2

tert-Butyl 2-(4-(cyano(4-(trifluoromethyl)phenyl)methylcarbamoyl)-1H-1,2,3-triazol-1-yl)ethylcarbamate was prepared in analogy to step 2 of the synthesis of Intermediate 3. from amino-(4-trifluoromethyl-phenyl)-acetonitrile hydrochloride and 1-(2-(tert-butoxycarbonylamino)ethyl)-1H-1,2,3-triazole-4-carboxylic acid. Light brown solid. LC/HR-MS: (M+H)⁺=439.1684.

Step 3

1-(2-Amino-ethyl)-1H-[1,2,3]triazole-4-carboxylic acid [cyano-(4-trifluoromethyl-phenyl)-methyl]-amide was prepared in analogy to step 3 of the synthesis of Intermediate 3 from tert-butyl 2-(4-(cyano(4-(trifluoromethyl)phenyl)methylcarbamoyl)-1H-1,2,3-triazol-1-yl)ethylcarbamate. Light brown solid. LC/HR-MS: (M+H)⁺=339.1170.

Intermediate 5

### Up-scaling of 1-(2-tert-Butoxycarbonylamino-ethyl)-1H-[1,2,3]triazole-4-carboxylic acid methyl ester by synthesis in flow

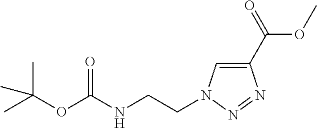 [SMILES: COC(=O)c1cn(CCNC(=O)OC(C)(C)C)nn1]

A stream of (2-azido-ethyl)-carbamic acid tert-butyl ester (11.25 g, 60.1 mmol) and copper(II) sulfate pentahydrate (1.2 ml, 1.2 mmol; 1 M aq. solution) in DMF (242 ml) was combined with a second stream containing methyl propiolate (7.58 g, 90.1 mmol) and sodium ascorbate (4.8 ml, 4.8 mmol, 1 M aq. solution) in DMF (242 ml) using a T-piece connector (reaction conducted on a Vapourtec R2+/R4 flow system). Both reagent streams were running at an individual flow rate of 0.25 ml/min, resulting in a total flow rate of 0.50 ml/min. The combined fluidic flow was then passed through 3×10 ml interlinked convection flow coils (CFC) made from PFA (perfluoroalkoxy) which were heated at 120° C. (residence time 1 h). A 250 psi back-pressure regulator with a protection guard (filled with glass wool) was used at the exit of the reactor to maintain system pressure. The reaction mixture was collected in a round bottom flask, the DMF removed under reduced pressure (<20 ml) and the crude reaction mixture suspended in water (100 ml). The aqueous phase was extracted with ethyl acetate (3×250 ml) and the combined organic phase back-washed with a sat. solution of sodium chloride (150 ml). The organic layer was dried over MgSO₄ and the solvent evaporated under reduced pressure. Purification of the crude reaction product over a short plug of silica gel (dichloromethane/EtOAc=7:3) and precipitation from cold hexane provided 12.22 g (75%) 1-(2-tert-butoxycarbonylamino-ethyl)-1H-[1,2,3]triazole-4-carboxylic acid methyl ester as an off-white solid. LC/HR-MS: (M+H)⁺=271.1396.

### Example 1

### 1-[2-(2,3,4-Trimethoxy-benzoylamino)-ethyl]-1H-[1,2,3]triazole-4-carboxylic acid [cyano-(4-ethyl-phenyl)-methyl]-amide

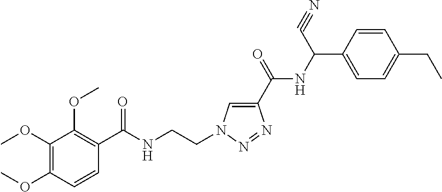 [SMILES: CCc1ccc(C(C#N)NC(=O)c2cn(CCNC(=O)c3ccc(OC)c(OC)c3OC)nn2)cc1]

To a cold (0° C.) solution of 1-(2-(2,3,4-trimethoxybenzamido)ethyl)-1H-1,2,3-triazole-4-carboxylic acid (65 mg, 186 μmol), Intermediate 2, and N-ethyldiisopropylamine (48.0 mg, 63.5 μl, 371 μmol,) in N,N-dimethylacetamide (5.88 ml) was added HATU (77.6 mg, 204 μmol). The reaction mixture was stirred at 0° C. for 1 h then 2-amino-2-(4-ethylphenyl)acetonitrile, hydrochloric salt (40.1 mg, 204 μmol), material of step 1 of the synthesis of Intermediate 3, was added and the reaction mixture was stirred overnight at room temperature. The reaction mixture was partitioned between 1M HCl and ethyl acetate. The organic layer was washed with a saturated aqueous solution of sodium hydrogenocarbonate then water and brine. The residue was purified by chromatography over silica gel (dichloromethane/AcOEt 100:0 to 60:40) to give the title compound, 36 mg (39.4%) as an off-white solid. LC/HR-MS: (M+H)⁺=493.2187.

### Example 2

### 1-[2-(2,3,4-Trimethoxy-benzoylamino)-ethyl]-1H-[1,2,3]triazole-4-carboxylic acid [cyano-(4-methylsulfanyl-phenyl)-methyl]-amide

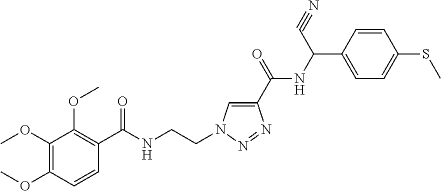

Step 1

2-Amino-2-(4-(methylthio)phenyl)acetonitrile hydrochloride was prepared from 4-methylsulfanyl-benzaldehyde in analogy to step 1 of the synthesis of Intermediate 3. Orange solid. GC-EI-MS: (M)⁺=178.

Step 2.

The title compound was prepared from 2-amino-2-(4-(methylthio)phenyl)acetonitrile, hydrochloride, and 1-(2-(2,3,4-trimethoxybenzamido)ethyl)-1H-1,2,3-triazole-4-carboxylic acid, Intermediate 2, in analogy to Example 1. White solid. LC/HR-MS: (M+H)⁺=511.1744.

### Example 3

### 1-[2-(2,3,4-Trimethoxy-benzoylamino)-ethyl]-1H-[1,2,3]triazole-4-carboxylic acid 4-methyl-benzylamide

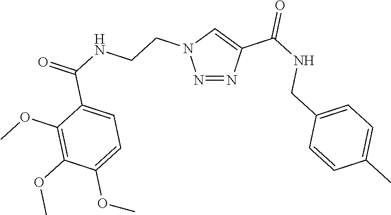

The title compound was prepared from 4-methyl-benzylamine and 1-(2-(2,3,4-trimethoxybenzamido)ethyl)-1H-1,2,3-triazole-4-carboxylic acid, Intermediate 2, in analogy to Example 1. White solid. LC/HR-MS: (M+H)⁺=454.2072.

### Example 4

### 1-[2-(2,3,4-Trimethoxy-benzoylamino)-ethyl]-1H-[1,2,3]triazole-4-carboxylic acid 3,4-dimethyl-benzylamide

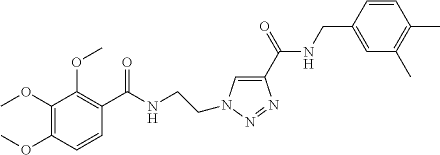

The title compound was prepared from 3,4-dimethyl-benzylamine and 1-(2-(2,3,4-trimethoxybenzamido)ethyl)-1H-1,2,3-triazole-4-carboxylic acid, Intermediate 2, in analogy to Example 1. White solid. LC/HR-MS: (M+H)⁺=468.2241.

### Example 5

### 1-[2-(2,3,4-Trimethoxy-benzoylamino)-ethyl]-1H-[1,2,3]triazole-4-carboxylic acid 4-chloro-benzylamide

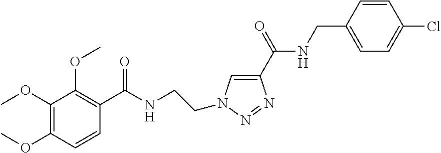 [SMILES: COc1ccc(C(=O)NCCn2cc(C(=O)NCc3ccc(Cl)cc3)nn2)c(OC)c1OC]

The title compound was prepared from 4-chloro-benzylamine and 1-(2-(2,3,4-trimethoxybenzamido)ethyl)-1H-1,2,3-triazole-4-carboxylic acid, Intermediate 2, in analogy to Example 1. White solid. LC/HR-MS: (M+H)⁺=474.1548.

### Example 6

### 1-[2-(2,3,4-Trimethoxy-benzoylamino)-ethyl]-1H-[1,2,3]triazole-4-carboxylic acid 4-methylsulfanyl-benzylamide

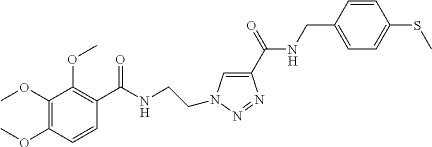

The title compound was prepared from 4-methylsulfanyl-benzylamine and 1-(2-(2,3,4-trimethoxybenzamido)ethyl)-1H-1,2,3-triazole-4-carboxylic acid, Intermediate 2, in analogy to Example 1. White solid. LC/HR-MS: (M+H)⁺=486.1807.

### Example 7

### 1-[2-(2,3,4-Trimethoxy-benzoylamino)-ethyl]-1H-[1,2,3]triazole-4-carboxylic acid 4-ethyl-benzylamide

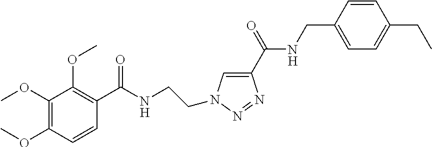 [SMILES: CCc1ccc(CNC(=O)c2cn(CCNC(=O)c3ccc(OC)c(OC)c3OC)nn2)cc1]

The title compound was prepared from 4-ethyl-benzylamine and 1-(2-(2,3,4-trimethoxybenzamido)ethyl)-1H-1,2,3-triazole-4-carboxylic acid, Intermediate 2, in analogy to Example 1. White solid. LC/HR-MS: (M+H)⁺=468.2227.

### Example 8

### 1-[2-(2,3,4-Trimethoxy-benzoylamino)-ethyl]-1H-[1,2,3]triazole-4-carboxylic acid [cyano-(4-methoxy-phenyl)-methyl]-amide

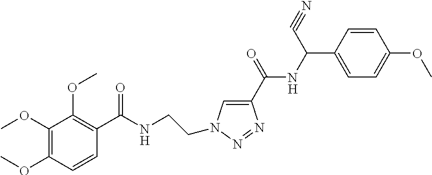

Step 1

Amino-(4-methoxy-phenyl)-acetonitrile hydrochloride was prepared from 4-methoxy-benzaldehyde in analogy to step 1 of the synthesis of Intermediate 3. Orange solid. GC-EI-MS: (M)⁺=162.

Step 2

The title compound was prepared from amino-(4-methoxy-phenyl)-acetonitrile, hydrochloride salt, and 1-(2-(2,3,4-trimethoxybenzamido)ethyl)-1H-1,2,3-triazole-4-carboxylic acid, Intermediate 2, in analogy to Example 1. Light yellow, amorphous solid.

LC/HR-MS: (M+H)⁺=495.1983.

### Example 9

### 1-[2-(2-Indan-2-yl-acetylamino)-ethyl]-1H-[1,2,3]triazole-4-carboxylic acid [cyano-(4-ethyl-phenyl)-methyl]-amide

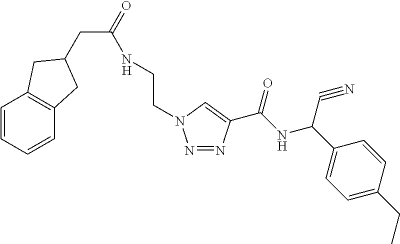 [SMILES: CCc1ccc(C(C#N)NC(=O)c2cn(CCNC(=O)CC3Cc4ccccc4C3)nn2)cc1]

Step 1

1-[2-(2-Indan-2-yl-acetylamino)-ethyl]-1H-E1,2,31triazole-4-carboxylic acid methyl ester was prepared in analogy to step 1 of synthesis of Intermediate 2, from 1-(2-amino-ethyl)-1H-[1,2,3]triazole-4-carboxylic acid methyl ester hydrochloride (Intermediate 1) and indan-2-yl-acetic acid as a brown solid that was used without further purification in the next step.

Step 2

1-(2-(2-(2,3-Dihydro-1H-inden-2-yl)acetamido)ethyl)-1H-1,2,3-triazole-4-carboxylic acid was prepared in analogy to step 2 of synthesis of Intermediate 2, from 1-(2-(2-indan-2-yl-acetylamino)-ethyl]-1H-[1,2,3]triazole-4-carboxylic acid methyl ester. Light yellow solid. LC/HR-MS: (M+H)⁺=315.1454.

Step 3

The title compound was prepared from 2-amino-2-(4-ethylphenyl)acetonitrile hydrochloride, product of step 1, Intermediate 3, and 1-(2-(2-(2,3-dihydro-1H-inden-2-yl)acetamido)ethyl)-1H-1,2,3-triazole-4-carboxylic acid in analogy to Example 1. Light yellow, amorphous solid. LC/HR-MS: (M+H)⁺=457.2351.

### Example 10

### 1-[2-(2,3,4-Trimethoxy-benzoylamino)-ethyl]-1H-[1,2,3]triazole-4-carboxylic acid [cyano-(4-trifluoromethoxy-phenyl)-methyl]-amide

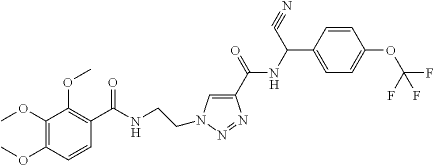 [SMILES: COc1ccc(C(=O)NCCn2cc(C(=O)NC(C#N)c3ccc(OC(F)(F)F)cc3)nn2)c(OC)c1OC]

Step 1

2-Amino-2-(4-(trifluoromethoxy)phenyl)acetonitrile hydrochloride was prepared from 4-trifluoromethoxy-benzaldehyde in analogy to step 1 of synthesis of Intermediate 3. White solid. GC-EI-MS: (M)⁺=216.

Step 2

The title compound was prepared from 2-amino-2-(4-(trifluoromethoxy)phenyl)acetonitrile hydrochloride and 1-(2-(2,3,4-trimethoxyb enzamido)ethyl)-1H-1,2,3-triazole-4-carboxylic acid, Intermediate 2, in analogy to Example 1. White solid. LC/HR-MS: (M+H)⁺=549.1701.

### Example 11

### 1-[2-(2-Methoxy-4-trifluoromethoxy-benzoylamino)-ethyl]-1H-[1,2,3]triazole-4-carboxylic acid [cyano-(4-ethyl-phenyl)-methyl]-amide

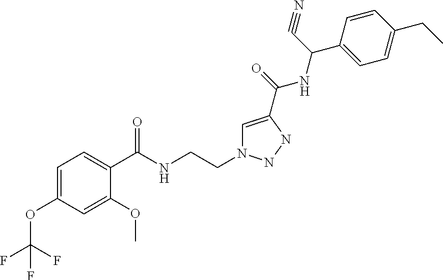 [SMILES: CCc1ccc(C(C#N)NC(=O)C2=CN(CCNC(=O)c3ccc(OC(F)(F)F)cc3OC)NN2)cc1]

To a cold (0° C.) solution of 2-methoxy-4-(trifluoromethoxy)-benzoic acid (47.5 mg, 201 μmol) and N-ethyldiisopropylamine (52 mg, 68.9 μl, 402 μmol,) in N,N-dimethylacetamide (6 ml) was added HATU (84.1 mg, 221 μmol). The reaction mixture was stirred at 0° C. for 1 h then 1-(2-amino-ethyl)-1H-[1,2,3]triazole-4-carboxylic acid [cyano-(4-ethyl-phenyl)-methyl]-amide (60 mg, 201 μmol), Intermediate 3, was added and the reaction mixture was stirred overnight at room temperature. The reaction mixture was partitioned between 1M HCl and ethyl acetate. The organic layer was washed with a saturated aqueous solution of sodium hydrogencarbonate then water and brine. The residue was purified by chromatography over silica gel (dichloromethane/AcOEt 100:0 to 60:40) to give the title compound, 104 mg (60.7%) as an off-white solid. LC/HR-MS: (M+H)⁺=517.1808.

### Example 12

### 1-{2-[(Indane-2-carbonyl)-amino]-ethyl}-1H-[1,2,3]triazole-4-carboxylic acid [cyano-(4-ethyl-phenyl)-methyl]-amide

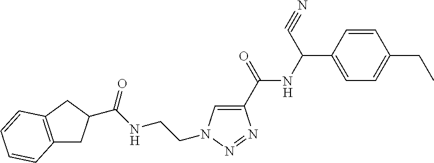 [SMILES: CCc1ccc(C(C#N)NC(=O)c2cn(CCNC(=O)C3Cc4ccccc4C3)nn2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and indan-2-carboxylic acid. White solid. LC/HR-MS: (M+H)⁺=443.2181.

### Example 13

### 1-{2-[(2,3-Dihydro-benzofuran-7-carbonyl)-amino]-ethyl}-1H-[1,2,3]triazole-4-carboxylic acid [cyano-(4-ethyl-phenyl)-methyl]-amide

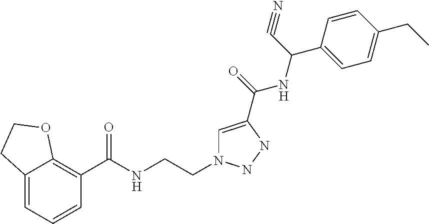 [SMILES: CCc1ccc(C(C#N)NC(=O)C2=CN(CCNC(=O)c3cccc4c3OCC4)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2,3-dihydrobenzofuran-7-carboxylic acid. Off-white solid. LC/HR-MS: (M+H)⁺=445.1795.

### Example 14

### 1-[2-(2,5-Dichloro-benzoylamino)-ethyl]-1H-[1,2,3]triazole-4-carboxylic acid [cyano-(4-ethyl-phenyl)-methyl]-amide

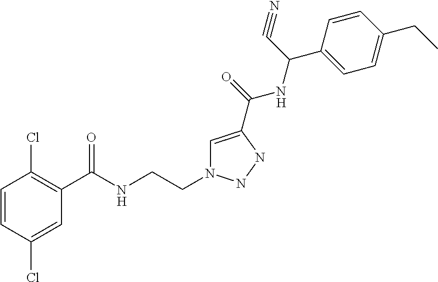 [SMILES: CCc1ccc(C(C#N)NC(=O)C2=CN(CCNC(=O)c3cc(Cl)ccc3Cl)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2,5-dichlorobenzoic acid. Off-white solid. LC/HR-MS: (M+H)⁺=471.1087.

### Example 15

### 6-Phenyl-pyridine-2-carboxylic acid [2-(4-{[cyano-(4-ethyl-phenyl)-methyl]-carbamoyl}-[1,2,3]triazol-1-yl)-ethyl]-amide

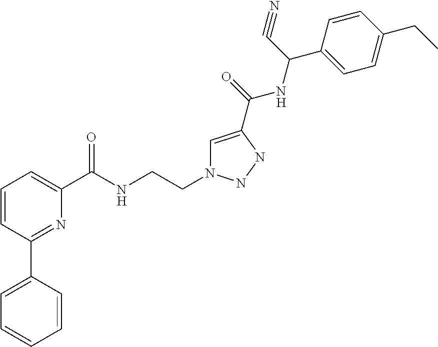 [SMILES: CCc1ccc(C(C#N)NC(=O)C2=CN(CCNC(=O)c3cccc(-c4ccccc4)n3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 6-phenylpyridine-2-carboxylic acid. Light yellow solid. LC/HR-MS: (M+H)⁺=480.2144.

### Example 16

### 1-[2-(2,3-Dimethoxy-benzoylamino)-ethyl]-1H-[1,2,3]triazole-4-carboxylic acid [cyano-(4-ethyl-phenyl)-methyl]-amide

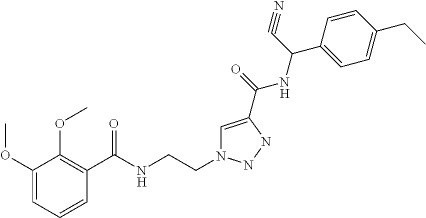 [SMILES: CCc1ccc(C(C#N)NC(=O)C2=CN(CCNC(=O)c3cccc(OC)c3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2,3-dimethoxy-benzoic acid. White solid. LC/HR-MS: (M+H)⁺=463.2090.

### Example 17

### 3-Methoxy-pyridine-2-carboxylic acid [2-(4-{[cyano-(4-ethyl-phenyl)-methyl]-carbamoyl}-[1,2,3]triazol-1-yl)-ethyl]-amide

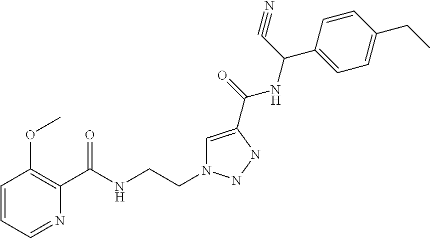 [SMILES: CCc1ccc(C(C#N)NC(=O)C2=CN(CCNC(=O)c3ncccc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 3-methoxy-pyridine-2-carboxylic acid. Off-white solid. LC/HR-MS: (M+H)⁺=434.1932.

### Example 18

### 1-{2-[(2,3-Dihydro-benzo[1,4]dioxine-5-carbonyl)-amino]-ethyl}-1H-[1,2,3]triazole-4-carboxylic acid [cyano-(4-ethyl-phenyl)-methyl]-amide

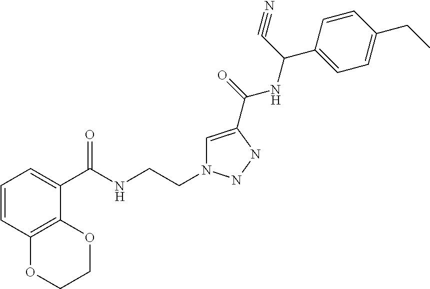 [SMILES: CCc1ccc(C(C#N)NC(=O)C2=CN(CCNC(=O)c3cccc4c3OCCO4)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2,3-dihydro-benzo[1,4]dioxine-5-carboxylic acid. White solid. LC/HR-MS: (M+H)⁺=461.1927.

### Example 19

### 1-[2-(2-Methoxy-4-methylsulfanyl-benzoylamino)-ethyl]-1H-[1,2,3]triazole-4-carboxylic acid [cyano-(4-ethyl-phenyl)-methyl]-amide

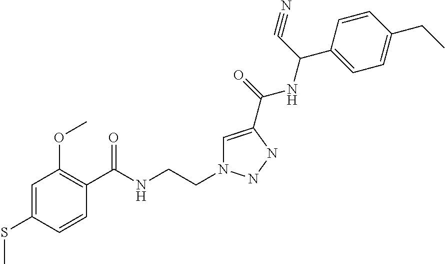 [SMILES: CCc1ccc(C(C#N)NC(=O)C2=CN(CCNC(=O)c3ccc(SC)cc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-methoxy-4-methylsulfanyl-benzoic acid. White solid. LC/HR-MS: (M+H)⁺=479.1861.

### Example 20

### 1-[2-(4-Chloro-2-methoxy-benzoylamino)-ethyl]-1H-[1,2,3]triazole-4-carboxylic acid [cyano-(4-ethyl-phenyl)-methyl]-amide

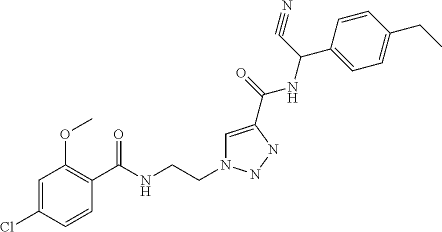 [SMILES: CCc1ccc(C(C#N)NC(=O)C2=CN(CCNC(=O)c3ccc(Cl)cc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 4-chloro-2-methoxy-benzoic acid. White solid. LC/HR-MS: (M+H)⁺=467.1577.

### Example 21

### 4-Chloro-6-methyl-pyridine-2-carboxylic acid [2-(4-{[cyano-(4-ethyl-phenyl)-methyl]-carbamoyl}-[1,2,3]triazol-1-yl)-ethyl]-amide

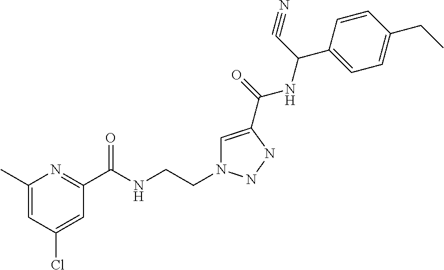 [SMILES: CCc1ccc(C(C#N)NC(=O)C2=CN(CCNC(=O)c3cc(Cl)cc(C)n3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 4-chloro-6-methyl-pyridine-2-carboxylic acid. Yellow solid. LC/HR-MS: (M+H)⁺=452.1589.

### Example 22

### 1-{2-[2-(2-Hydroxy-indan-2-yl)-acetylamino]-ethyl}-1H-[1,2,3]triazole-4-carboxylic acid [cyano-(4-ethyl-phenyl)-methyl]-amide

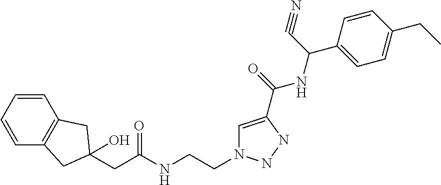 [SMILES: CCc1ccc(C(C#N)NC(=O)C2=CN(CCNC(=O)CC3(O)Cc4ccccc4C3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and (2-hydroxy-indan-2-yl)-acetic acid. White solid. LC/HR-MS: (M+H)⁺=473.2290.

### Example 23

### 1-[2-(2-Methoxy-4-trifluoromethyl-benzoylamino)-ethyl]-1H-[1,2,3]triazole-4-carboxylic acid [cyano-(4-ethyl-phenyl)-methyl]-amide

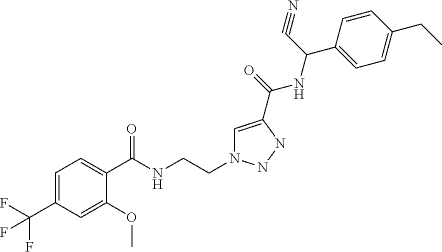 [SMILES: CCc1ccc(C(C#N)NC(=O)C2=CN(CCNC(=O)c3ccc(C(F)(F)F)cc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-methoxy-4-trifluoromethyl-benzoic acid. White solid. LC/HR-MS: (M+H)⁺=501.1849.

### Example 24

### 1-[2-(2-Trifluoromethoxy-benzoylamino)-ethyl]-1H-[1,2,3]triazole-4-carboxylic acid [cyano-(4-ethyl-phenyl)-methyl]-amide

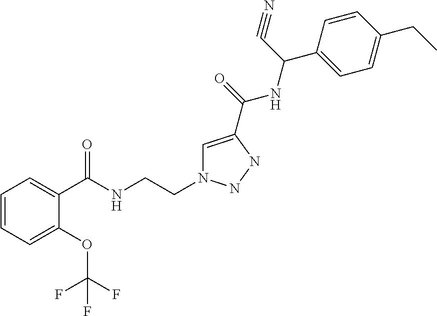 [SMILES: CCc1ccc(C(C#N)NC(=O)C2=CN(CCNC(=O)c3ccccc3OC(F)(F)F)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-trifluoromethyl-benzoic acid. Off-white solid. LC/HR-MS: (M+H)⁺=487.1702.

### Example 25

### 1-{2-[(3-Methyl-1H-indene-2-carbonyl)-amino]-ethyl}-1H-[1,2,3]triazole-4-carboxylic acid [cyano-(4-ethyl-phenyl)-methyl]-amide

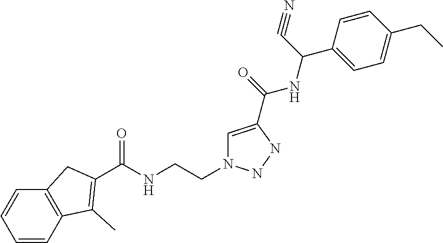 [SMILES: CCc1ccc(C(C#N)NC(=O)C2=CN(CCNC(=O)C3=C(C)c4ccccc4C3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 3-methyl-1H-indene-2-carboxylic acid. Off-white solid. LC/HR-MS: (M+H)⁺=455.2173.

### Example 26

### 6-Methoxy-pyridine-2-carboxylic acid [2-(4-{[cyano-(4-ethyl-phenyl)-methyl]-carbamoyl}-[1,2,3]triazol-1-yl)-ethyl]-amide

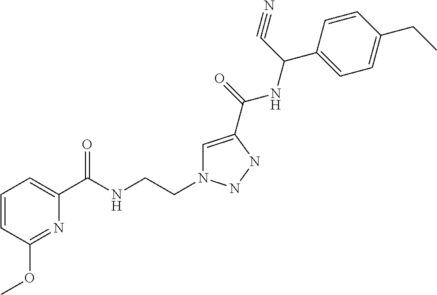 [SMILES: CCc1ccc(C(C#N)NC(=O)C2=CN(CCNC(=O)c3cccc(OC)n3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 6-methoxypyridine-2-carboxylic acid. White solid. LC/HR-MS: (M+H)⁺=434.1937.

### Example 27

### 4-Chloro-pyridine-2-carboxylic acid [2-(4-{[cyano-(4-ethyl-phenyl)-methyl]-carbamoyl}-[1,2,3]triazol-1-yl)-ethyl]-amide

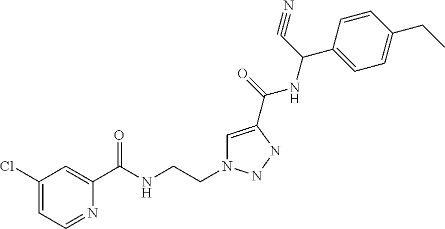 [SMILES: CCc1ccc(C(C#N)NC(=O)C2=CN(CCNC(=O)c3cc(Cl)ccn3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 4-chloropicolinic acid. Off-white solid. LC/HR-MS: (M+H)⁺=438.1435.

### Example 28

### 5-Ethoxy-pyridine-2-carboxylic acid [2-(4-{[cyano-(4-ethyl-phenyl)-methyl]-carbamoyl}-[1,2,3]triazol-1-yl)-ethyl]-amide

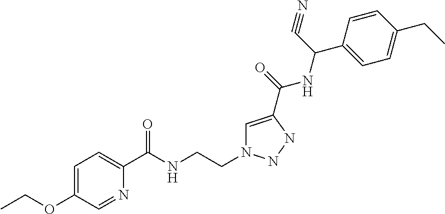 [SMILES: CCOc1ccc(C(=O)NCCN2C=C(C(=O)NC(C#N)c3ccc(CC)cc3)NN2)nc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 5-ethoxy-pyridine-2-carboxylic acid. White solid. LC/HR-MS: (M+H)⁺=448.2089.

### Example 29

### N-[2-(4-{[Cyano-(4-ethyl-phenyl)-methyl]-carbamoyl}-[1,2,3]triazol-1-yl)-ethyl]-2-methoxy-nicotinamide

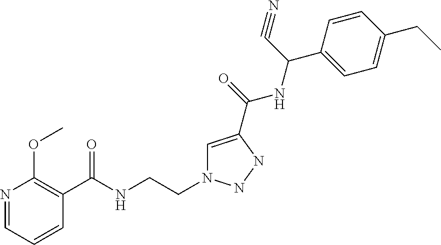 [SMILES: CCc1ccc(C(C#N)NC(=O)C2=CN(CCNC(=O)c3cccnc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-methoxy-nicotinic acid. White solid. LC/HR-MS: (M+H)⁺=434.1939.

### Example 30

### 5-Bromo-N-[2-(4-{[cyano-(4-ethyl-phenyl)-methyl]-carbamoyl}-[1,2,3]triazol-1-yl)-ethyl]-4-methoxy-nicotinamide

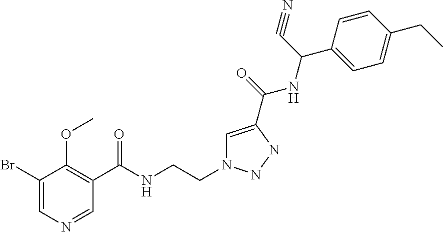 [SMILES: CCc1ccc(C(C#N)NC(=O)C2=CN(CCNC(=O)c3cncc(Br)c3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 5-bromo-4-methoxy-nicotinic acid. Light yellow solid. LC/HR-MS: (M+H)⁺=512.1031.

### Example 31

### 1-[2-(4-Chloro-2-fluoro-benzoylamino)-ethyl]-1H-[1,2,3]triazole-4-carboxylic acid [cyano-(4-ethyl-phenyl)-methyl]-amide

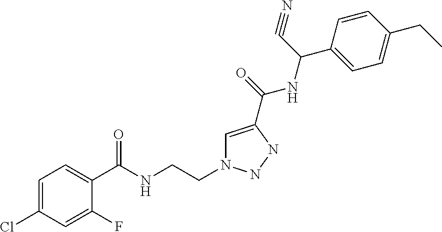 [SMILES: CCc1ccc(C(C#N)NC(=O)C2=CN(CCNC(=O)c3ccc(Cl)cc3F)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 4-chloro-2-fluoro-benzoic acid. White solid. LC/HR-MS: (M+H)⁺=455.1376.

### Example 32

### 1-[2-(2-Fluoro-5-methoxy-benzoylamino)-ethyl]-1H-[1,2,3]triazole-4-carboxylic acid [cyano-(4-ethyl-phenyl)-methyl]-amide

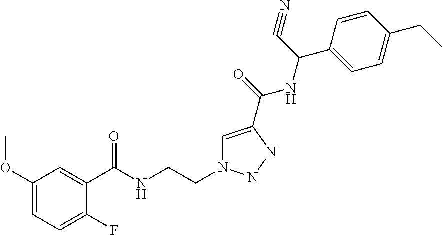 [SMILES: CCc1ccc(C(C#N)NC(=O)C2=CN(CCNC(=O)c3cc(OC)ccc3F)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-fluoro-5-methoxybenzoic acid. Off-white solid. LC/HR-MS: (M+H)⁺=451.1885.

### Example 33

### 1-[2-(2-Methoxymethyl-benzoylamino)-ethyl]-1H-[1,2,3]triazole-4-carboxylic acid [cyano-(4-ethyl-phenyl)-methyl]-amide

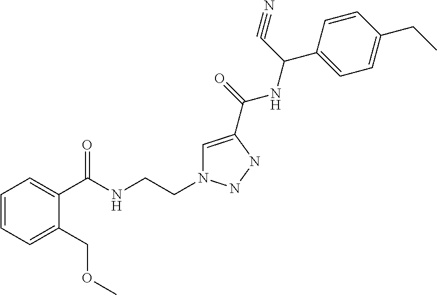 [SMILES: CCc1ccc(C(C#N)NC(=O)C2=CN(CCNC(=O)c3ccccc3COC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-methoxymethyl-benzoic acid. White solid. LC/HR-MS: (M+H)⁺=447.2137.

### Example 34

### N-[2-(4-{[Cyano-(4-ethyl-phenyl)-methyl]-carbamoyl}-[1,2,3]triazol-1-yl)-ethyl]-2-methoxy-6-methyl-nicotinamide

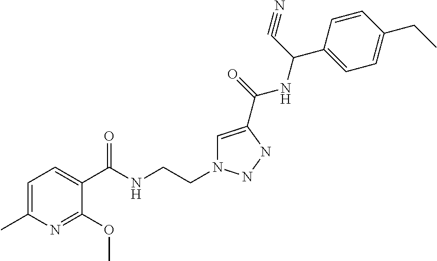 [SMILES: CCc1ccc(C(C#N)NC(=O)C2=CN(CCNC(=O)c3ccc(C)nc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-methoxy-6-methyl-nicotinic acid. White solid. LC/HR-MS: (M+H)⁺=448.2077.

### Example 35

### N-[2-(4-{[Cyano-(4-ethyl-phenyl)-methyl]-carbamoyl}-[1,2,3]triazol-1-yl)-ethyl]-2,6-dimethoxy-nicotinamide

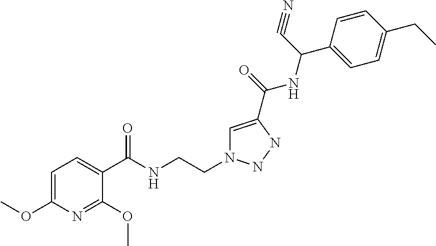 [SMILES: CCc1ccc(C(C#N)NC(=O)C2=CN(CCNC(=O)c3ccc(OC)nc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2,6-dimethoxy-nicotinic acid. White solid. LC/HR-MS: (M+H)⁺=464.2027.

### Example 36

### 1-[2-(4-Fluoro-2-methoxy-benzoylamino)-ethyl]-1H-[1,2,3]triazole-4-carboxylic acid [cyano-(4-ethyl-phenyl)-methyl]-amide

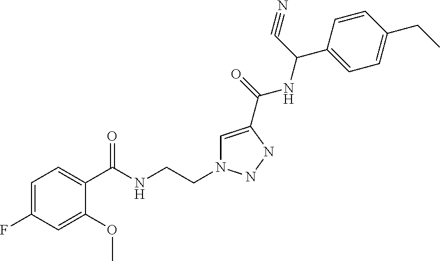 [SMILES: CCc1ccc(C(C#N)NC(=O)C2=CN(CCNC(=O)c3ccc(F)cc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 4-fluoro-2-methoxybenzoic acid. White solid. LC/HR-MS: (M+H)⁺=451.1889.

### Example 37

### 1-[2-(2-Fluoro-5-trifluoromethyl-benzoylamino)-ethyl]-1H-[1,2,3]triazole-4-carboxylic acid [cyano-(4-ethyl-phenyl)-methyl]-amide

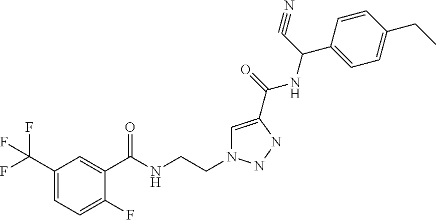 [SMILES: CCc1ccc(C(C#N)NC(=O)C2=CN(CCNC(=O)c3cc(C(F)(F)F)ccc3F)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-fluoro-5-trifluoromethyl-benzoic acid. Light brown solid. LC/HR-MS: (M+H)⁺=489.1647.

### Example 38

### 1-[2-(2-Fluoro-4-trifluoromethyl-benzoylamino)-ethyl]-1H-[1,2,3]triazole-4-carboxylic acid [cyano-(4-ethyl-phenyl)-methyl]-amide

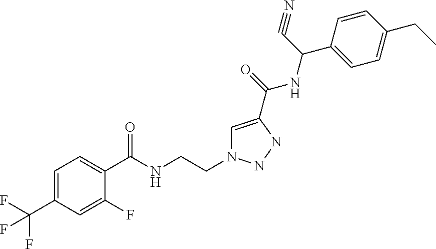 [SMILES: CCc1ccc(C(C#N)NC(=O)C2=CN(CCNC(=O)c3ccc(C(F)(F)F)cc3F)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-fluoro-4-trifluoromethyl-benzoic acid. Light brown solid. LC/HR-MS: (M+H)⁺=489.1646.

### Example 39

### 1-[2-(2-Methoxy-5-trifluoromethyl-benzoylamino)-ethyl]-1H-[1,2,3]triazole-4-carboxylic acid [cyano-(4-ethyl-phenyl)-methyl]-amide

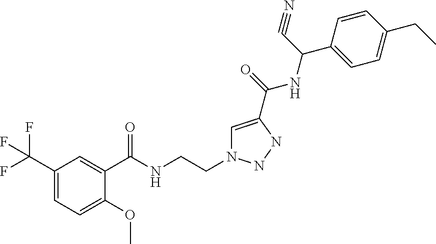 [SMILES: CCc1ccc(C(C#N)NC(=O)C2=CN(CCNC(=O)c3cc(C(F)(F)F)ccc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-methoxy-5-(trifloromethyl)-benzoic acid. Light yellow solid. LC/HR-MS: (M+H)⁺=501.1853.

### Example 40

### 6-Methoxy-pyridazine-3-carboxylic acid [2-(4-{[cyano-(4-ethyl-phenyl)-methyl]-carbamoyl}-[1,2,3]triazol-1-yl)-ethyl]-amide

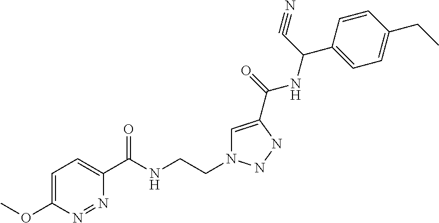 [SMILES: CCc1ccc(C(C#N)NC(=O)C2=CN(CCNC(=O)c3ccc(OC)nn3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 6-methoxypyridazine-3-carboxylic-acid. White solid. LC/HR-MS: (M+H)⁺=435.1882.

### Example 41

### 1-[2-(2-Chloro-4-methanesulfonyl-benzoylamino)-ethyl]-1H-[1,2,3]triazole-4-carboxylic acid [cyano-(4-ethyl-phenyl)-methyl]-amide

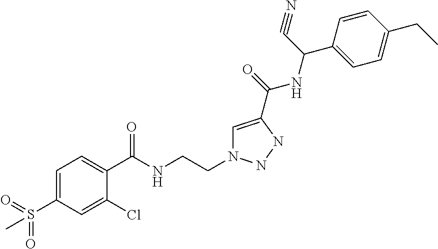 [SMILES: CCc1ccc(C(C#N)NC(=O)C2=CN(CCNC(=O)c3ccc(S(C)(=O)=O)cc3Cl)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 2-chloro-4-methanesulfonyl-benzoic acid. White solid. LC/HR-MS: (M+H)⁺=515.1280.

### Example 42

### 1-[2-(5-Methanesulfonyl-2-methoxy-benzoylamino)-ethyl]-1H-[1,2,3]triazole-4-carboxylic acid [cyano-(4-ethyl-phenyl)-methyl]-amide

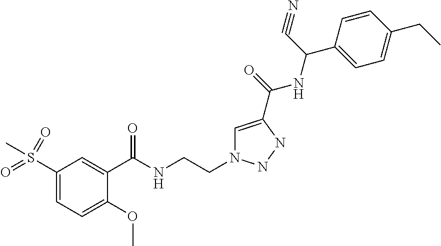 [SMILES: CCc1ccc(C(C#N)NC(=O)C2=CN(CCNC(=O)c3cc(S(C)(=O)=O)ccc3OC)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 5-methanesulfonyl-2-methoxy-benzoic acid. White solid. LC/HR-MS: (M+H)⁺=511.1762.

### Example 43

### 5-(4-Chloro-phenoxy)-pyridine-2-carboxylic acid [2-(4-{[cyano-(4-ethyl-phenyl)-methyl]-carbamoyl}-[1,2,3]triazol-1-yl)-ethyl]-amide

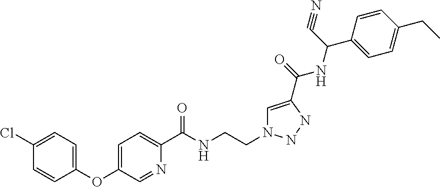 [SMILES: CCc1ccc(C(C#N)NC(=O)C2=CN(CCNC(=O)c3ccc(Oc4ccc(Cl)cc4)cn3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 5-(4-chloro-phenoxy)-pyridine-2-carboxylic acid. White solid. LC/HR-MS: (M+H)⁺=530.1698.

### Example 44

### 5-Chloro-pyridine-2-carboxylic acid [2-(4-{[cyano-(4-ethyl-phenyl)-methyl]-carbamoyl}-[1,2,3]triazol-1-yl)-ethyl]-amide

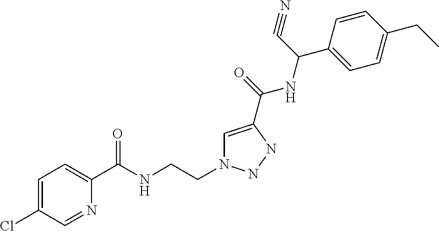 [SMILES: CCc1ccc(C(C#N)NC(=O)C2=CN(CCNC(=O)c3ccc(Cl)cn3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 5-chloro-pyridine-2-carboxylic acid. White solid. LC/HR-MS: (M+H)⁺=438.1444.

### Example 45

### 5-Trifluoromethyl-pyridine-2-carboxylic acid [2-(4-{[cyano-(4-ethyl-phenyl)-methyl]-carbamoyl}-[1,2,3]triazol-1-yl)-ethyl]-amide

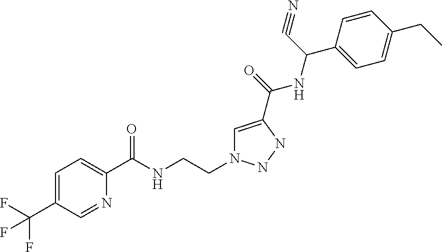 [SMILES: CCc1ccc(C(C#N)NC(=O)C2=CN(CCNC(=O)c3ccc(C(F)(F)F)cn3)NN2)cc1]

The title compound was obtained in analogy to Example 11 from 1-(2-amino-ethyl)-1H-[1,2,3]triazole-4-carboxylic acid [cyano-(4-ethyl-phenyl)-methyl]-amide, Intermediate 3, and 5-trifluoromethyl-pyridine-2-carboxylic acid. White solid. LC/HR-MS: (M+H)⁺=472.1699.

### Example 46

### 1-[2-(1-Oxo-1,3-dihydro-isoindol-2-yl)-ethyl]-1H-[1,2,3]triazole-4-carboxylic acid [cyano-(4-ethyl-phenyl)-methyl]-amide

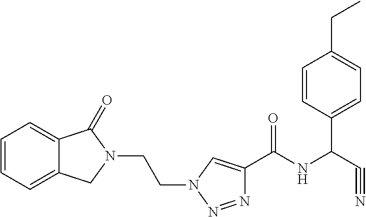 [SMILES: CCc1ccc(C(C#N)NC(=O)c2cn(CCN3Cc4ccccc4C3=O)nn2)cc1]

Step 1

Methyl 2-(bromomethyl)benzoate (670 mg, 2.92 mmol) in DMSO (25 ml) was treated at RT under argon with potassium carbonate (1.01 g, 7.31 mmol) then 1-(2-amino-ethyl)-1H[1,2,3]triazole-4-carboxylic acid methyl ester hydrochloride (604 mg, 2.92 mmol),

Intermediate 1, was added and the mixture was then stirred at 65° C. for 18 h. It was cooled to room temperature and partitioned between water and AcOEt. The layers were separated and the organic layer was dried over sodium sulfate, filtered and concentrated. The residue was chromatographed over 10 g silica gel with AcOEt/dichloromethane (gradient 0-40% AcOEt). All fractions containing product were combined and concentrated in vacuo to give 1-[2-(1-oxo-1,3-dihydro-isoindol-2-yl)-ethyl]-1H-[1,2,3]triazole-4-carboxylic acid methyl ester, 140 mg (16.7%) as white solid. LC/HR-MS: (M+H)⁺=287.1143.

Step 2

1-[2-(1-Oxo-1,3-dihydro-isoindol-2-yl)-ethyl]-1H-[1,2,3]triazole-4-carboxylic acid methyl ester (155 mg, 541 □mol) in methanol (13.8 ml) was treated at room temperature under argon with 1M aqueous NaOH (2.17 ml, 2.17 mmol) and the mixture was stirred for 2 h at room temperature until completion of the reaction according to TLC analysis. The mixture was concentrated in vacuo and the residue partitioned between 1M HCl and EtOAc. The aqueous layer was extracted with methylene chloride, the combined organic layers were washed with water, dried over sodium sulphate, filtered and concentrated to give 1-[2-(1-oxo-1,3-dihydro-isoindol-2-yl)-ethyl]-1H-[1,2,3]triazole-4-carboxylic acid as a white solid (77 mg, 52.2%) which was used without further purification in the next step.

Step 3

The title compound was prepared in analogy to Example 1 from 1-[2-(1-oxo-1,3-dihydro-isoindol-2-yl)-ethyl]-1H-[1,2,3]triazole-4-carboxylic acid and 2-amino-2-(4-ethylphenyl)acetonitrile hydrochloride (material of Intermediate 3, step 1) as a white solid. LC/HR-MS: (M+H)⁺=415.1874.

### Example 47

### 1-[2-(5-Methoxy-1-oxo-1,3-dihydro-isoindol-2-yl)-ethyl]-1H-[1,2,3]triazole-4-carboxylic acid [cyano-(4-ethyl-phenyl)-methyl]-amide

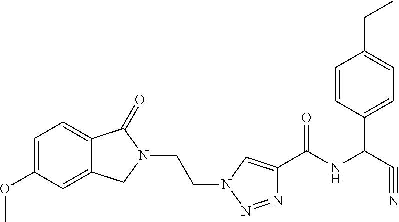 [SMILES: CCc1ccc(C(C#N)NC(=O)c2cn(CCN3Cc4cc(OC)ccc4C3=O)nn2)cc1]

Step 1

1-[2-(5-Methoxy-1-oxo-1,3-dihydro -isoindol-2-yl)-ethyl]-1H-[1,2,3]triazole-4-carboxylic acid methyl ester was prepared in analogy to Example 46, step 1 from methyl 2-(bromomethyl)-4-methoxybenzoate and 1-(2-amino-ethyl)-1H[1,2,3]triazole-4-carboxylic acid methyl ester hydrochloride, Intermediate 1. White solid. LC/HR-MS: (M+H)⁺=317.1249.

Step 2

1-[2-(5-Methoxy-1-oxo-1,3-dihydro-isoindol-2-yl)-ethyl]-1H-[1,2,3]triazole-4-carboxylic acid was prepared in analogy to Example 46 step 2 from 1-[2-(5-methoxy-1-oxo-1,3-dihydro-isoindol-2-yl)-ethyl]-1H-[1,2,3]triazole-4-carboxylic acid methyl ester as white solid which was used without further purification in the next step.

Step 3

The title compound was prepared in analogy to Example 1 from 1-[2-(5-methoxy-1-oxo-1,3-dihydro-isoindol-2-yl)-ethyl]-1H-[1,2,3]triazole-4-carboxylic acid and 2-amino-2-(4-ethylphenyl)acetonitrile hydrochloride (material of Intermediate 3, step 1). Off-white solid. LC/HR-MS: (M+H)⁺=445.1979.

### Example 48

### 1-[2-(2,3,4-Trimethoxy-benzoylamino)-ethyl]-1H-[1,2,3]triazole-4-carboxylic acid (benzo[b]thiophen-5-yl-cyano-methyl)-amide

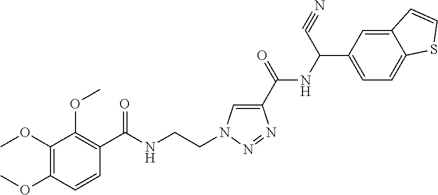 [SMILES: COc1ccc(C(=O)NCCn2cc(C(=O)NC(C#N)c3ccc4sccc4c3)nn2)c(OC)c1OC]

Step 1

A solution of 1-benzothiophen-5-carbaldehyde (890 mg, 5.487 mmol), trimethylsilanecarbonitrile (980 mg, 1.24 ml, 9.88 mmol) and zinc(II) iodide (4.38 mg, 13.7 μmol) in methanol (15 mL) was stirred 15 min at room temperature. The reaction mixture was then transferred into a pressure-resistant vial. Aqueous ammonia (7M, 23.5 ml, 165 mmol) was added and the reaction mixture was stirred at 40° C. for 2 h. The solvent was evaporated and the residue suspended in diethylether (200 mL) and filtered. A solution of hydrochloric acid in dioxane (4M) was added. The precipitate was filtered, washed with diethyl ether and dried in vacuo to afford 1.034 g (84%) of amino-benzo[b]thiophen-5-yl-acetonitrile hydrochloride as a light yellow solid. MS+(m/z): 205.4 (M+NH₄)⁺.

Step 2

To a cold (0° C.) solution of 1-(2-(2,3,4-trimethoxybenzamido)ethyl)-1H-1,2,3-triazole-4-carboxylic acid (100 mg, 285 μmol, Intermediate 2) and N-ethyldiisopropylamine (111 mg, 147 μl, 856 μmol) in N,N-dimethylacetamide (9.23 ml) was added 2-(1H-7-azabenzotriazol-1-yl)-1,1,3,3-tetramethyl uronium hexafluorophosphate methanaminium (119 mg, 314 μmol). The reaction mixture was stirred lh at room temperature then amino-benzo[b]thiophen-5-yl-acetonitrile hydrochloride (64.1 mg, 285 μmol) was added and the reaction mixture was stirred overnight at room temperature. The reaction mixture was partitioned between 1M HCl and ethyl acetate. The organic layer was washed with a saturated aqueous solution of sodium hydrogencarbonate then water and brine. The residue was purified by chromatography over silica gel (silica gel 20 g, dichloromethane/AcOEt 100:0 to 60:40). One fraction was isolated and dried in vacuo, affording 110 mg (74%) of the title compound as a white solid. MS+(m/z): 521.3 (M+H)⁺.

### Example 49

### 1-[2-(2,3,4-Trimethoxy-benzoylamino)-ethyl]-1H-[1,2,3]triazole-4-carboxylic acid (cyano-naphthalen-2-yl-methyl)-amide

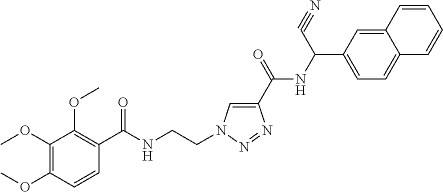 [SMILES: COc1ccc(C(=O)NCCn2cc(C(=O)NC(C#N)c3ccc4ccccc4c3)nn2)c(OC)c1OC]

Step 1

Amino-naphthalen-2-yl-acetonitrile hydrochloride was prepared from 2-naphthaldehyde in analogy to Example 48, step 1. Yellow solid. MS−(m/z): 218.4 (M)⁻

Step 2

The title compound was prepared from amino-naphthalen-2-yl-acetonitrile hydrochloride and 1-(2-(2,3,4-trimethoxybenzamido)ethyl)-1H-1,2,3-triazole-4-carboxylic acid (Intermediate 2) in analogy to Example 48, step 2. White solid. MS+(m/z): 515.4 (M+H)⁺.

### Examples 50 and 51

### 1-[2-(2,3,4-Trimethoxy-benzoylamino)-ethyl]-1H-[1,2,3]triazole-4-carboxylic acid [(R)-cyano-(4-ethyl-phenyl)-methyl]-amide

### and

### 1-[2-(2,3,4-Trimethoxy-benzoylamino)-ethyl]-1H-[1,2,3]triazole-4-carboxylic acid [(S)-cyano-(4-ethyl-phenyl)-methyl]-amide

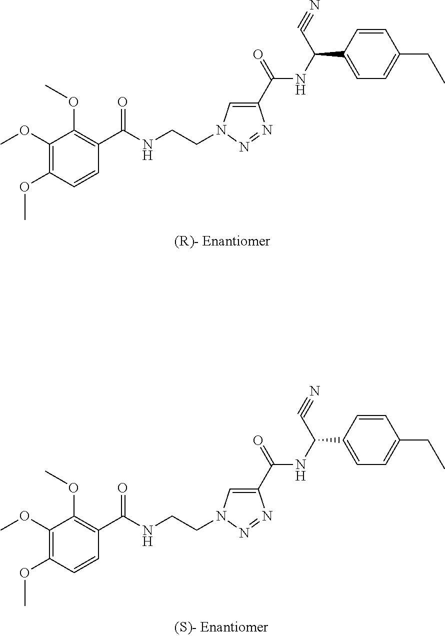

A sample of the racemic mixture of N-(cyano(4-ethylphenyl)methyl)-1-(2-(2,3,4-trimethoxybenzamido)ethyl)-1H-1,2,3-triazole-4-carboxamide (136 mg), obtained in Example 1, was separated by chiral preparative HPLC (Chiralpak AD, eluent: 40% isopropanol / heptane) to give the title compounds as white solids, respectively. Retention times for the two enantiomers: 181 minutes (56.7 mg, LC/HR-MS: (M+H)⁺=493.2193) and 280 minutes (58.7 mg, LC/HR-MS: (M+H)⁺=493.2196). Stereochemical assignments were made based on correlation of biological activity with proposed binding mode as determined by molecular modeling and have not been unequivocally proven.

### Example 52

### 1-[2-(2,3,4-Trimethoxy-benzoylamino)-ethyl]-1H-[1,2,3]triazole-4-carboxylic acid [cyano-(3-methoxy-phenyl)-methyl]-amide

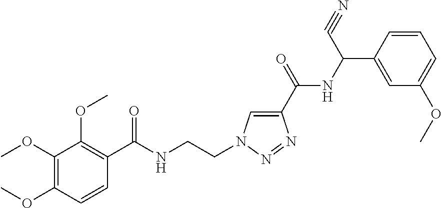 [SMILES: COc1cccc(C(C#N)NC(=O)c2cn(CCNC(=O)c3ccc(OC)c(OC)c3OC)nn2)c1]

Step 1

Amino-(3-methoxy-phenyl)-acetonitrile hydrochloride was prepared from 3-methoxy-benzaldehydehyde in analogy to Example 48, step 1. Yellow solid. MS+(m/z): 199.5 (M+H)⁺.

Step 2

The title compound was prepared from amino-(3-methoxy-phenyl)-acetonitrile hydrochloride and 1-(2-(2,3,4-trimethoxyb enzamido)ethyl)-1H-1,2,3-triazole-4-carboxylic acid (Intermediate 2) in analogy to Example 48, step 2. White solid. MS+(m/z): 495.4 (M+H)⁺.

### Example 53

### 1-[2-(2,3,4-Trimethoxy-benzoylamino)-ethyl]-1H-[1,2,3]triazole-4-carboxylic acid (benzo[1,3]dioxol-5-yl-cyano-methyl)-amide

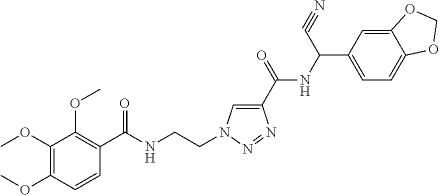 [SMILES: COc1ccc(C(=O)NCCn2cc(C(=O)NC(C#N)c3ccc4c(c3)OCO4)nn2)c(OC)c1OC]

Step 1

Amino-benzo[1,3]dioxol-5-yl-acetonitrile hydrochloride was prepared from piperonal in analogy to Example 48, step 1. Light red solid.

Step 2

The title compound was prepared from amino-benzo[1,3]dioxol-5-yl-acetonitrile, hydrochloride and 1-(2-(2,3,4-trimethoxybenzamido)ethyl)-1H-1,2,3-triazole-4-carboxylic acid (Intermediate 2, step 2) in analogy to Example 48, step 2. White solid. MS+(m/z): 509.4 (M+H)⁺.

### Example 54

### 1-[2-(2,3,4-Trimethoxy-benzoylamino)-ethyl]-1H-[1,2,3]triazole-4-carboxylic acid [(4-bromo-phenyl)-cyano-methyl]-amide

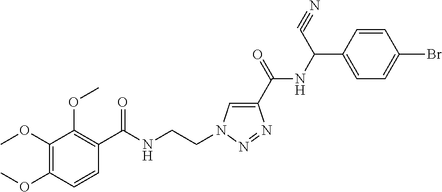 [SMILES: COc1ccc(C(=O)NCCn2cc(C(=O)NC(C#N)c3ccc(Br)cc3)nn2)c(OC)c1OC]

Step 1

Amino-(4-bromo-phenyl)-acetonitrile hydrochloride was prepared from 4-bromobenzaldehyde in analogy to Example 48, step 1. Yellow solid.

Step 2

The title compound was prepared from amino-(4-bromo-phenyl)-acetonitrile hydrochloride and 1-(2-(2,3,4-trimethoxybenzamido)ethyl)-1H-1,2,3-triazole-4-carboxylic acid (Intermediate 2) in analogy to Example 48, step 2. White solid. MS+(m/z): 543.3 (M)⁺.

### Example 55

### 1-[2-(2,3,4-Trimethoxy-benzoylamino)-ethyl]-1H-[1,2,3]triazole-4-carboxylic acid [cyano-(3-methylsulfanyl-phenyl)-methyl]-amide

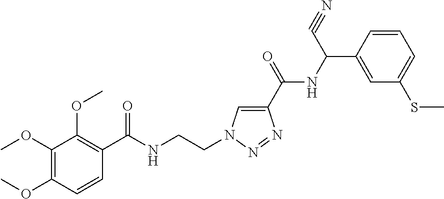

Step 1

Amino-(3-methylsulfanyl-phenyl)-acetonitrile hydrochloride was prepared from 3-methylsulfanyl-benzaldehyde in analogy to Example 48, step 1. Light yellow solid. MS−(m/z): 214.4 (M-H)⁻.

Step 2

The title compound was prepared from amino-(3-methylsulfanyl-phenyl)-acetonitrile hydrochloride and 1-[2-(2,3 ,4-trimethoxyb enzamido)ethyl)-1H-1,2,3-triazole-4-carboxylic acid (Intermediate 2) in analogy to Example 48, step 2. Yellow solid. MS+(m/z): 511.4 (M)⁺.

### Example 56

### 1-[2-(2,3,4-Trimethoxy-benzoylamino)-ethyl]-1H-[1,2,3]triazole-4-carboxylic acid [(4-benzyloxy-phenyl)-cyano-methyl]-amide

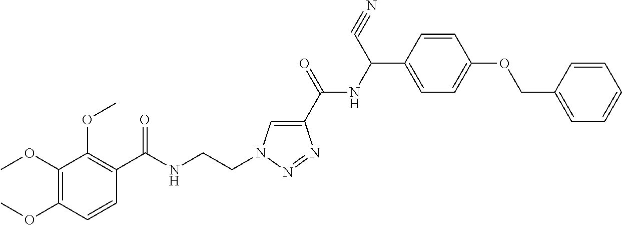 [SMILES: COc1ccc(C(=O)NCCn2cc(C(=O)NC(C#N)c3ccc(OCc4ccccc4)cc3)nn2)c(OC)c1OC]

Step 1

Amino-(4-benzyloxy-phenyl)-acetonitrile hydrochloride was prepared from 4-benzoxybenzaldehyde in analogy to Example 48, step 1. Light brown solid. MS−(m/z): 273.4 (M-H)⁻.

Step 2

The title compound was prepared from amino-(4-benzyloxy-phenyl)-acetonitrile hydrochloride and 1-(2-(2,3,4-trimethoxybenzamido)ethyl)-1H-1,2,3-triazole-4-carboxylic acid (Intermediate 2) in analogy to Example 48, step 2. White solid. MS+(m/z): 571.5 (M+H)⁺.

### Example 57

### 1-[2-(2,3,4-Trimethoxy-benzoylamino)-ethyl]-1H-[1,2,3]triazole-4-carboxylic acid (cyano-naphthalen-1-yl-methyl)-amide

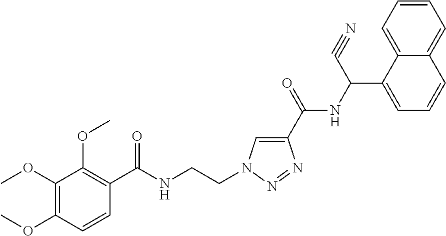 [SMILES: COc1ccc(C(=O)NCCn2cc(C(=O)NC(C#N)c3cccc4ccccc34)nn2)c(OC)c1OC]

Step 1

Amino-naphthalen-1-yl-acetonitrile hydrochloride was prepared from 1-naphthaldehyde in analogy to Example 48, step 1. Yellow solid. MS+(m/z): 279.4 (M+MeCN+H)⁺.

Step 2

The title compound was prepared from amino-naphthalen-1-yl-acetonitrile hydrochloride and 1-(2-(2,3,4-trimethoxybenzamido)ethyl)-1H-1,2,3-triazole-4-carboxylic acid (Intermediate 2) in analogy to Example 48, step 2. Yellow solid. MS+(m/z): 515.5 (M+H)⁺.

### Example 58

### 1-[2-(2,3,4-Trimethoxy-benzoylamino)-ethyl]-1H-[1,2,3]triazole-4-carboxylic acid [cyano-(4-cyclopropyl-phenyl)-methyl]-amide

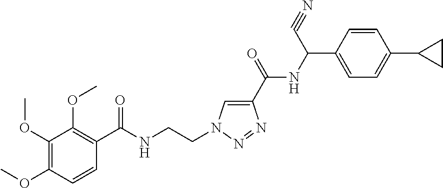 [SMILES: COc1ccc(C(=O)NCCn2cc(C(=O)NC(C#N)c3ccc(C4CC4)cc3)nn2)c(OC)c1OC]

Step 1

Amino-(4-cyclopropyl-phenyl)-acetonitrile hydrochloride was prepared from 4-cyclopropylbenzaldehyde in analogy to step 1 of the synthesis of Intermediate 3. Light brown solid. GC-EI-MS: ((M−H +Si(CH₃)₃)⁺=244.

Step 2

The title compound was prepared from amino-(4-cyclopropyl-phenyl)-acetonitrile hydrochloride and 1-(2-(2,3,4-trimethoxybenzamido)ethyl)-1H-1,2,3-triazole-4-carboxylic acid (Intermediate 2) in analogy to Example 1. Off white solid. LC/HR-MS: (M+H)⁺=505.2193.

### Example 59

### 1-[2-(2,3,4-Trimethoxy-benzoylamino)-ethyl]-1H-[1,2,3]triazole-4-carboxylic acid [(2-bromo-phenyl)-cyano-methyl]-amide

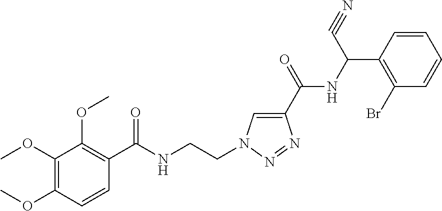 [SMILES: COc1ccc(C(=O)NCCn2cc(C(=O)NC(C#N)c3ccccc3Br)nn2)c(OC)c1OC]

Step 1

Amino-(2-bromo-phenyl)-acetonitrile hydrochloride was prepared from 2-bromobenzaldehyde in analogy to Example 48, step 1. Light yellow solid.

Step 2

The title compound was prepared from amino-(2-bromo-phenyl)-acetonitrile hydrochloride and 1-(2-(2,3,4-trimethoxybenzamido)ethyl)-1H-1,2,3-triazole-4-carboxylic acid (Intermediate 2) in analogy to Example 48, step 2. Light yellow solid. MS+(m/z): 543.3 (M+H)⁺.

### Example 60

### 1-[2-(2,3,4-Trimethoxy-benzoylamino)-ethyl]-1H-[1,2,3]triazole-4-carboxylic acid [cyano-(4-trifluoromethyl-phenyl)-methyl]-amide

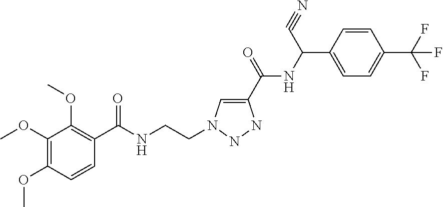 [SMILES: COc1ccc(C(=O)NCCN2C=C(C(=O)NC(C#N)c3ccc(C(F)(F)F)cc3)NN2)c(OC)c1OC]

The title compound was obtained in analogy to Example 11 from 1-(2-Amino-ethyl)-1H-[1,2,31triazole-4-carboxylic acid [cyano-(4-trifluoromethyl-phenyl)-methyl]-amide, Intermediate 4, and 2,3,4 trimethoxybenzoic acid. White solid. LC/HR-MS: (M+H)⁺=533.1757.

### Example 61

### 1-[2-(2,3,4-Trimethoxy-benzoylamino)-ethyl]-1H-[1,2,3]triazole-4-carboxylic acid [cyano-(4-isopropyl-phenyl)-methyl]-amide

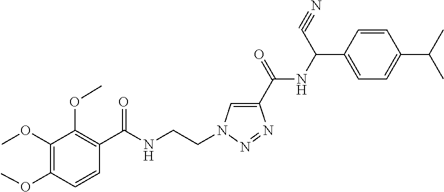 [SMILES: COc1ccc(C(=O)NCCn2cc(C(=O)NC(C#N)c3ccc(C(C)C)cc3)nn2)c(OC)c1OC]

Step 1

Amino-(4-isopropyl-phenyl)-acetonitrile hydrochloride was prepared from cuminaldehyde in analogy to Example 48, step 1. Light yellow solid.

Step 2

The title compound was prepared from amino-(4-isopropyl-phenyl)-acetonitrile hydrochloride and 1-(2-(2,3,4-trimethoxybenzamido)ethyl)-1H-1,2,3-triazole-4-carboxylic acid (Intermediate 2) in analogy to Example 48, step 2. Light yellow solid. MS+(m/z): 507.5 (M+H)⁺.

### Example 62

### 1-[2-(2,3,4-Trimethoxy-benzoylamino)-ethyl]-1H-[1,2,3]triazole-4-carboxylic acid (cyano-p-tolyl-methyl)-amide

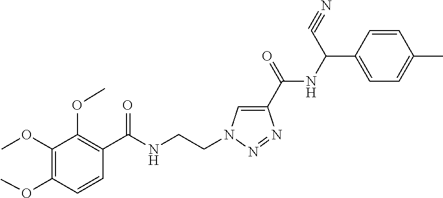

Step 1

Amino-p-tolyl-acetonitrile hydrochloride was prepared from para-tolualdehyde in analogy to Example 48, step 1. Yellow solid.

Step 2

The title compound was prepared from amino-p-tolyl-acetonitrile hydrochloride and 1-(2-(2,3,4-trimethoxybenzamido)ethyl)-1H-1,2,3-triazole-4-carboxylic acid (Intermediate 2) in analogy to Example 48, step 2. Brown oil. MS+(m/z): 479.5 (M+H)⁺.

### Example 63

### 1-[2-(2,3,4-Trimethoxy-benzoylamino)-ethyl]-1H-[1,2,3]triazole-4-carboxylic acid [cyano-(5-ethyl-pyridin-2-yl)-methyl]-amide

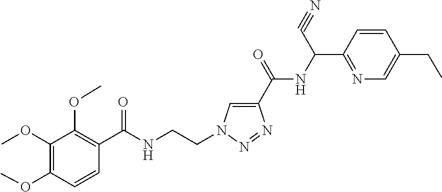 [SMILES: CCc1ccc(C(C#N)NC(=O)c2cn(CCNC(=O)c3ccc(OC)c(OC)c3OC)nn2)nc1]

Step 1

Amino-(5-ethyl-pyridin-2-yl)-acetonitrile hydrochloride was prepared from 5-ethyl-2-pyridinecarbaldehyde in analogy to Example 48, step 1. Red solid.

Step 2

The title compound was prepared from amino-(5-ethyl-pyridin-2-yl)-acetonitrile hydrochloride and 1-(2-(2,3,4-trimethoxyb enzamido)ethyl)-1H-1,2,3-triazole-4-carboxylic acid (Intermediate 2) in analogy to Example 48, step 2. Yellow oil. MS+(m/z): 494.5 (M+H)⁺.

### Example 64

### 1-[2-(2,3,4-Trimethoxy-benzoylamino)-ethyl]-1H-[1,2,3]triazole-4-carboxylic acid [cyano-(4-propyl-phenyl)-methyl]-amide

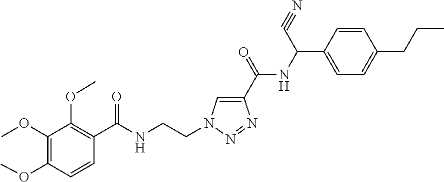 [SMILES: CCCc1ccc(C(C#N)NC(=O)c2cn(CCNC(=O)c3ccc(OC)c(OC)c3OC)nn2)cc1]

Step 1

Amino-(4-propyl-phenyl)-acetonitrile hydrochloride was prepared from 4-propylbenzaldehyde in analogy to Example 48, step 1. Light yellow solid.

Step 2

The title compound was prepared from amino-(4-propyl-phenyl)-acetonitrile hydrochloride and 1-(2-(2,3,4-trimethoxybenzamido)ethyl)-1H-1,2,3-triazole-4-carboxylic acid (Intermediate 2) in analogy to Example 48, step 2. White solid. MS+(m/z): 507.7 (M+H)⁺.

### Example 65

### 1-[2-(2,3,4-Trimethoxy-benzoylamino)-ethyl]-1H-[1,2,3]triazole-4-carboxylic acid [cyano-(6-methoxy-pyridin-3-yl)-methyl]-amide

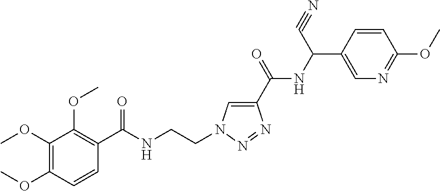

Step 1

Amino-(6-methoxy-pyridin-3-yl)-acetonitrile hydrochloride was prepared from 2-methoxy pyridine-5-carboxaldehyde in analogy to Example 48, step 1. Light yellow solid.

Step 2

The title compound was prepared from amino-(6-methoxy-pyridin-3-yl)-acetonitrile hydrochloride and 1-(2-(2,3,4-trimethoxybenzamido)ethyl)-1H-1,2,3-triazole-4-carboxylic acid (Intermediate 2) in analogy to Example 48, step 2. Yellow oil. MS+(m/z): 496.4 (M+H)⁺.

### Example 66

### 1-[2-(2,3,4-Trimethoxy-benzoylamino)-ethyl]-1H-[1,2,3]triazole-4-carboxylic acid (benzo[b]thiophen-6-yl-cyano-methyl)-amide

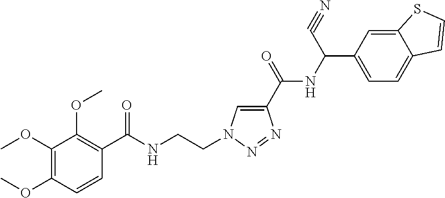 [SMILES: COc1ccc(C(=O)NCCn2cc(C(=O)NC(C#N)c3ccc4ccsc4c3)nn2)c(OC)c1OC]

Step 1

In a 25 mL three-necked flask, magnesium (257 mg, 10.6 mmol) was suspended in THF,(1.7 mL) and heated to reflux. A solution of 6-bromobenzo[b]thiophene (1.5 g, 7.04 mmol) in THF (8.3 mL) was added dropwise. The reaction mixture was stirred at reflux for 2 h, then cooled to 0° C. N,N-Dimethylformamide (1.03 g, 1.09 ml, 14.1 mmol) was added dropwise and the reaction allowed to warm up to room temperature. The resulting green suspension was stirred overnight then concentrated. The residue was purified by chromatography over silica gel. One fraction was isolated and dried in vacuo, affording 821 mg (72%) of benzo[b]thiophene-6-carbaldehyde as a yellow oil.

Step 2

Amino-benzo[b]thiophen-6-yl-acetonitrile hydrochloride was prepared from benzo[b]thiophene-6-carbaldehyde in analogy to Example 48, step 1. Brown solid.

Step 3

The title compound was prepared from amino-benzo[b]thiophen-6-yl-acetonitrile hydrochloride and 1-(2-(2,3,4-trimethoxyb enzamido)ethyl)-1H-1,2,3-triazole-4-carboxylic acid (Intermediate 2) in analogy to Example 48, step 2. Yellow solid. MS+(m/z): 521.5 (M+H)⁺.

### Example 67

### 1-[2-(2,3,4-Trimethoxy-benzoylamino)-ethyl]-1H-[1,2,3]triazole-4-carboxylic acid [cyano-(4-isopropoxy-phenyl)-methyl]-amide

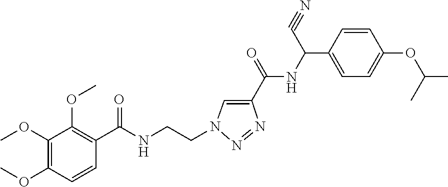 [SMILES: COc1ccc(C(=O)NCCn2cc(C(=O)NC(C#N)c3ccc(OC(C)C)cc3)nn2)c(OC)c1OC]

Step 1

Amino-(4-isopropoxy-phenyl)-acetonitrile hydrochloride was prepared from 4-isopropoxybenzaldehyde in analogy to Example 48, step 1. Red oil.

Step 2

The title compound was prepared from amino-(4-isopropoxy-phenyl)-acetonitrile, hydrochloride and 1-(2-(2,3,4-trimethoxybenzamido)ethyl)-1H-1,2,3-triazole-4-carboxylic acid (Intermediate 2) in analogy to Example 48, step 2. Light yellow oil. MS+(m/z): 523.6 (M+H)⁺.

### Example 68

### 1-[2-(2,3,4-Trimethoxy-benzoylamino)-ethyl]-1H-[1,2,3]triazole-4-carboxylic acid [(4-chloro-phenyl)-cyano-methyl]-amide

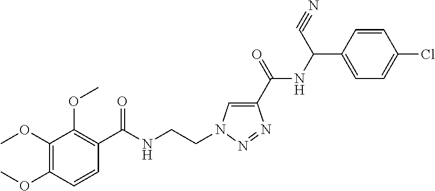 [SMILES: COc1ccc(C(=O)NCCn2cc(C(=O)NC(C#N)c3ccc(Cl)cc3)nn2)c(OC)c1OC]

Step 1

Amino-(4-chloro-phenyl)-acetonitrile hydrochloride was prepared from 4-chlorbenzaldehyde in analogy to Example 48, step 1. Light green solid.

Step 2

The title compound was prepared from amino-(4-chloro-phenyl)-acetonitrile hydrochloride and 1-(2-(2,3,4-trimethoxybenzamido)ethyl)-1H-1,2,3-triazole-4-carboxylic acid (Intermediate 2) in analogy to Example 48, step 2. Yellow oil. MS+(m/z): 499.5 (M+H)⁺.

### Example 69

### 1-[2-(2,5-Dichloro-benzoylamino)-ethyl]-1H-[1,2,3]triazole-4-carboxylic acid [cyano-(4-trifluoromethyl-phenyl)-methyl]-amide

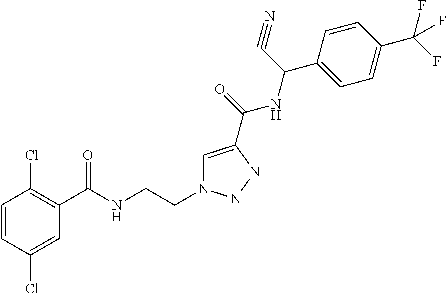 [SMILES: N#CC(NC(=O)C1=CN(CCNC(=O)c2cc(Cl)ccc2Cl)NN1)c1ccc(C(F)(F)F)cc1]

The title compound was obtained in analogy to Example 11 from 1-(2-Amino-ethyl)-1H-[1,2,3]triazole-4-carboxylic acid [cyano-(4-trifluoromethyl-phenyl)-methyl]-amide, Intermediate 4, and 2,5dichloro-benzoic acid. Off-white solid. LC/HR-MS: (M−H)⁻=509.0514.

### Example 70

### 1-{2-[(3-Methyl-1H-indene-2-carbonyl)-amino]-ethyl}-1H-[1,2,3]triazole-4-carboxylic acid [cyano-(4-trifluoromethyl-phenyl)-methyl]-amide

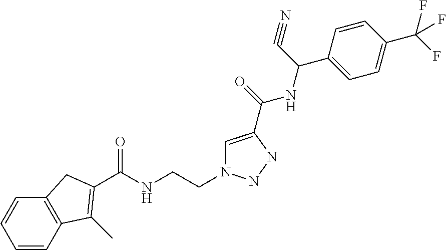 [SMILES: CC1=C(C(=O)NCCN2C=C(C(=O)NC(C#N)c3ccc(C(F)(F)F)cc3)NN2)Cc2ccccc21]

The title compound was obtained in analogy to Example 11 from 1-(2-Amino-ethyl)-1H-[1,2,3]triazole-4-carboxylic acid [cyano-(4-trifluoromethyl-phenyl)-methyl]-amide, Intermediate 4, and 3-methyl-1H-indene-2-carboxylic acid. White solid. LC/HR-MS: (M+H)⁺=495.1744.

### Example 71

### 1-[2-(2-Methoxy-4-trifluoromethoxy-benzoylamino)-ethyl]-1H-[1,2,3]triazole-4-carboxylic acid [cyano-(4-trifluoromethyl-phenyl)-methyl]-amide

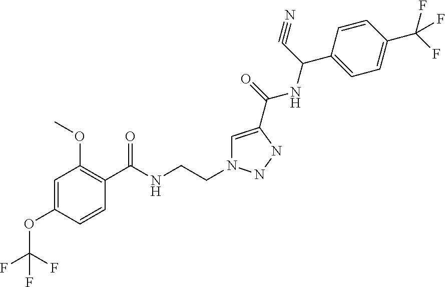 [SMILES: COc1cc(OC(F)(F)F)ccc1C(=O)NCCN1C=C(C(=O)NC(C#N)c2ccc(C(F)(F)F)cc2)NN1]

The title compound was obtained in analogy to Example 11 from 1-(2-Amino-ethyl)-1H-[1,2,3]triazole-4-carboxylic acid [cyano-(4-trifluoromethyl-phenyl)-methyl]-amide, Intermediate 4, and 2-methoxy-4-(trifluoromethoxy)benzoic acid. Light yellow solid. LC/HR-MS: (M+H)⁺=557.1358.

### Example 72

### 1-[2-(2-Methoxy-4-trifluoromethyl-benzoylamino)-ethyl]-1H-[1,2,3]triazole-4-carboxylic acid [cyano-(4-trifluoromethyl-phenyl)-methyl]-amide

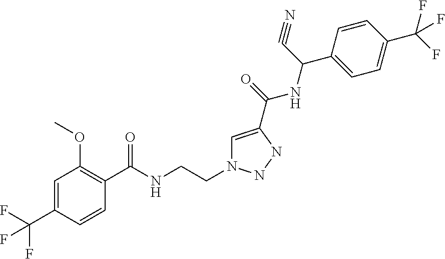 [SMILES: COc1cc(C(F)(F)F)ccc1C(=O)NCCN1C=C(C(=O)NC(C#N)c2ccc(C(F)(F)F)cc2)NN1]

The title compound was obtained in analogy to Example 11 from 1-(2-Amino-ethyl)-1H-[1,2,3]triazole-4-carboxylic acid [cyano-(4-trifluoromethyl-phenyl)-methyl]-amide, Intermediate 4, and 2-methoxy-4-(trifluoromethyl)benzoic acid. Light yellow solid. LC/HR-MS: (M−H)⁻=539.1271.

### Example 73

### 1-[2-(2-Fluoro-4-trifluoromethyl-benzoylamino)-ethyl]-1H-[1,2,3]triazole-4-carboxylic acid [cyano-(4-trifluoromethyl-phenyl)-methyl]-amide

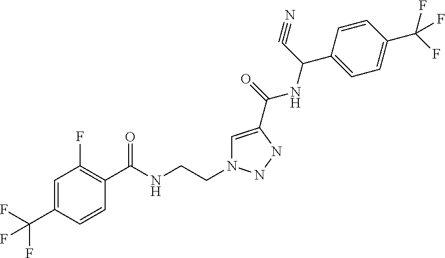 [SMILES: N#CC(NC(=O)C1=CN(CCNC(=O)c2ccc(C(F)(F)F)cc2F)NN1)c1ccc(C(F)(F)F)cc1]

The title compound was obtained in analogy to Example 11 from 1-(2-Amino-ethyl)-1H-[1,2,3]triazole-4-carboxylic acid [cyano-(4-trifluoromethyl-phenyl)-methyl]-amide, Intermediate 4, and 2-fluoro-4-(trifluoromethyl)benzoic acid. Off-white solid. LC/HR-MS: (M−H)⁻=527.1071.

### Example 74

### 1-[2-(2-Fluoro-4-methoxy-benzoylamino)-ethyl]-1H-[1,2,3]triazole-4-carboxylic acid [cyano-(4-trifluoromethyl-phenyl)-methyl]-amide

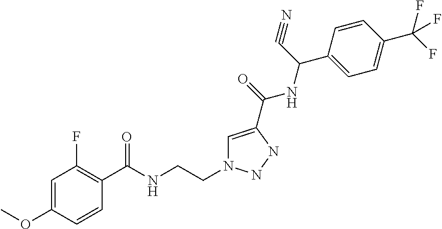 [SMILES: COc1ccc(C(=O)NCCN2C=C(C(=O)NC(C#N)c3ccc(C(F)(F)F)cc3)NN2)c(F)c1]

The title compound was obtained in analogy to Example 11 from 1-(2-Amino-ethyl)-1H-[1,2,3]triazole-4-carboxylic acid [cyano-(4-trifluoromethyl-phenyl)-methyl]-amide, Intermediate 4, and 2-fluoro-4-methoxy-benzoic acid. White solid. LC/HR-MS: (M+H)⁺=491.1450.

### Example 75

### 1-[2-(2-Fluoro-4-methyl-benzoylamino)-ethyl]-1H-[1,2,3]triazole-4-carboxylic acid [cyano-(4-trifluoromethyl-phenyl)-methyl]-amide

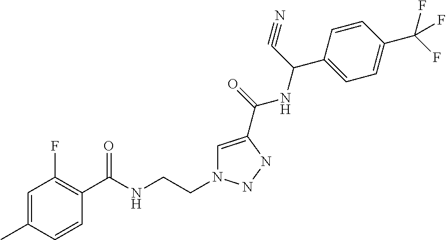 [SMILES: Cc1ccc(C(=O)NCCN2C=C(C(=O)NC(C#N)c3ccc(C(F)(F)F)cc3)NN2)c(F)c1]

The title compound was obtained in analogy to Example 11 from 1-(2-Amino-ethyl)-1H-[1,2,3]triazole-4-carboxylic acid [cyano-(4-trifluoromethyl-phenyl)-methyl]-amide, Intermediate 4, and 2-fluoro-4-methyl-benzoic acid. White solid. LC/HR-MS: (M+H)⁺=475.1499.

### Example 76

### 1-[2-(2-Fluoro-4-trifluoromethyl-benzoylamino)-ethyl]-1H-[1,2,3]triazole-4-carboxylic acid [cyano-(4-cyclopropyl-phenyl)-methyl]-amide

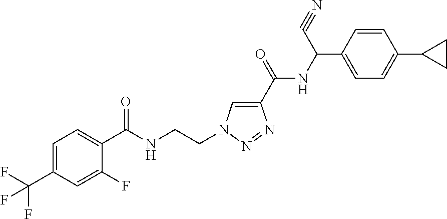 [SMILES: N#CC(NC(=O)c1cn(CCNC(=O)c2ccc(C(F)(F)F)cc2F)nn1)c1ccc(C2CC2)cc1]

Step 1

Methyl 1-(2-(2-fluoro-4-(trifluoromethyl)benzamido)ethyl)-1H-1,2,3-triazole-4-carboxylate was prepared in analogy to step 1 of the synthesis of Intermediate 2 from 1-(2-amino-ethyl)-1H-[1,2,3]triazole-4-carboxylic acid methyl ester hydrochloride, Intermediate 1, and 2-fluoro-4-trifluoromethyl-benzoic acid. Off-white solid. LC/HR-MS: (M+H)⁺=361.0921.

Step 2

1-(2-(2-Fluoro -4-(trifluoromethyl)benzamido)ethyl)-1H-1,2,3-triazole-4-carboxylic acid was prepared in analogy to step 2 of the synthesis of Intermediate 2 from methyl 1-(2-(2-fluoro-4-(trifluoromethyl)benzamido)ethyl)-1H-1,2,3-triazole-4-carboxylate. White solid. LC/HR-MS: (M+H)⁺=347.0760.

Step 3

The title compound was prepared in analogy to Example 1 from 1-(2-(2-fluoro-4-(trifluoromethyl)benzamido)ethyl)-1H-1,2,3-triazole-4-carboxylic acid and amino-(4-cyclopropyl-phenyl)-acetonitrile hydrochloride, product of Example 58, step 1. White solid. LC/HR-MS: (M+H)⁺=501.1658.

### Example 77

### 1-[2-(2,3,4-Trimethoxy-benzoylamino)-ethyl]-1H-[1,2,3]triazole-4-carboxylic acid [2-(4-methoxy-phenyl)-2-oxo-ethyl]-amide

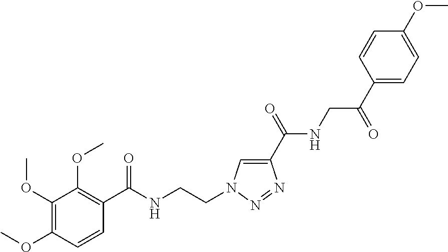

The title compound was prepared in analogy to Example 1 from 1-[2-(2,3,4-trimethoxy-benzoylamino)-ethyl]-1H-[1,2,3]triazole-4-carboxylic acid, intermediate 2, and 2-amino-1-(4-methoxy-phenyl)-ethanone, hydrochloride salt. Off-white solid. LC/HR-MS: (M+H)⁺=498.1980.

### Example 78

### 1-]2-(2-Fluoro-4-trifluoromethyl-benzoylamino)-ethyl]-1H-[1,2,3]triazole-4-carboxylic acid (3-oxo-3-phenyl-propyl)-amide

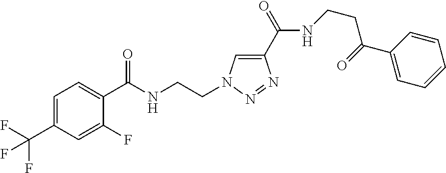 [SMILES: O=C(CCNC(=O)c1cn(CCNC(=O)c2ccc(C(F)(F)F)cc2F)nn1)c1ccccc1]

The title compound was prepared in analogy to Example 1 from 3-amino-1-phenyl-propan-1-one, hydrochloride salt, and 1-(2-(2-fluoro-4-(trifluoromethyl)benzamido)ethyl)-1H-1,2,3-triazole-4-carboxylic acid, product of step 2 of Example 76. White solid. LC/HR-MS: (M+H)⁺=478.1499.

### Example 79

### 1-[2-(2-Fluoro-4-trifluoromethyl-benzoylamino)-ethyl]-1H-[1,2,3]triazole-4-carboxylic acid (1-methyl-3-phenyl-propyl)-amide

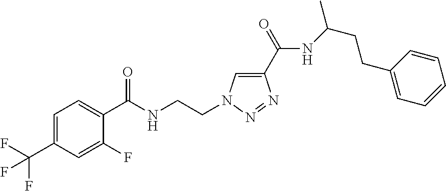 [SMILES: CC(CCc1ccccc1)NC(=O)c1cn(CCNC(=O)c2ccc(C(F)(F)F)cc2F)nn1]

The title compound was prepared in analogy to Example 1 from 1-methyl-3-phenyl-propylamine and 1-(2-(2-fluoro-4-(trifluoromethyl)benzamido)ethyl)-1H-1,2,3-triazole-4-carboxylic acid, product of Example 76 step 2. White solid. LC/HR-MS: (M+H)⁺=478.1866.

### Example 80

### (S)-4-Phenyl-2-({1-[2-(2,3,4-trimethoxy-benzoylamino)-ethyl]-1H-[1,2,3]triazole-4-carbonyl}-amino)-butyric acid ethyl ester

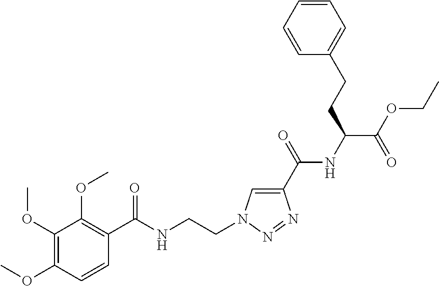 [SMILES: CCOC(=O)[C@H](CCc1ccccc1)NC(=O)c1cn(CCNC(=O)c2ccc(OC)c(OC)c2OC)nn1]

The title compound was prepared in analogy to Example 1 from 142-(2,3,4-trimethoxy-benzoylamino)-ethyl]-1H-[1,2,3]triazole-4-carboxylic acid, Intermediate 2, and (S)-2-amino-4-phenyl-butyric acid ethyl ester, hydrochloride salt. Light yellow solid. LC/HR-MS: (M+H)⁺=540.2452.

### Example 81

### (S)-2-({1-[2-(2-Fluoro-4-trifluoromethyl-benzoylamino)-ethyl]-1H-[1,2,3]triazole-4-carbonyl}-amino)-4-phenyl-butyric acid ethyl ester

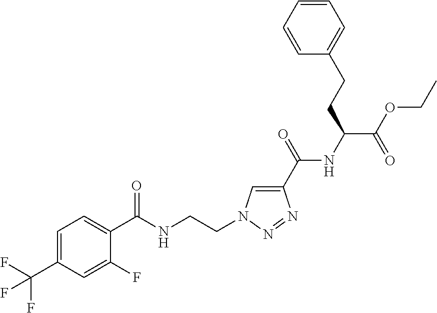 [SMILES: CCOC(=O)[C@H](CCc1ccccc1)NC(=O)c1cn(CCNC(=O)c2ccc(C(F)(F)F)cc2F)nn1]

The title compound was prepared in analogy to Example 1 from 1-(2-(2-fluoro-4-(trifluoromethyl)benzamido)ethyl)-1H-1,2,3-triazole-4-carboxylic acid, product of Example step 2 of 76, and (S)-2-amino-4-phenyl-butyric acid ethyl ester, hydrochloride salt. White solid. LC/HR-MS: (M+H)⁺=536.1917.

### Example 82

### (S)-2-({1-[2-(2-Fluoro-4-trifluoromethyl-benzoylamino)-ethyl]-1H-[1,2,3]triazole-4-carbonyl}-amino)-4-phenyl-butyric acid

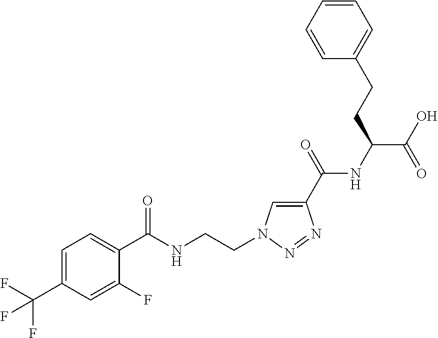 [SMILES: O=C(N[C@@H](CCc1ccccc1)C(=O)O)c1cn(CCNC(=O)c2ccc(C(F)(F)F)cc2F)nn1]

(S)-Ethyl 2-(1-(2-(2-fluoro-4-(trifluoromethyl)benzamido)ethyl)-1H-1,2,3-triazole-4-carboxamido)-4-phenylbutanoate (50 mg 93.4 □mol), product of Example 81, in ethanol was treated at room temperature under argon with 1M aqueous NaOH (373 □l, 373 □mol) and the mixture was stirred for 12 h at room temperature. The reaction mixture was concentrated in vacuo and the residue partitioned between 1N HCl and AcOEt. The organic layer was dried over sodium sulfate, filtered and the filtrate was concentrated in vacuo to give the title compound as a white solid (45 mg, 95%). LC/HR-MS: (M+H)⁺=508.1607.

### Example 83

### (S)-4-Phenyl-2-({1-[2-(2,3,4-trimethoxy-benzoylamino)-ethyl]-1H-[1,2,3]triazole-4-carbonyl}-amino)-butyric acid

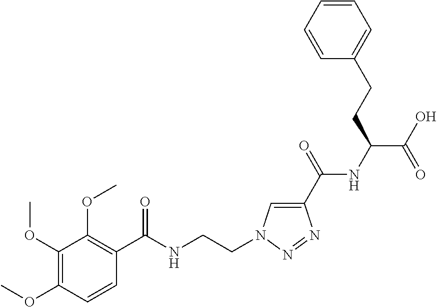 [SMILES: COc1ccc(C(=O)NCCn2cc(C(=O)N[C@@H](CCc3ccccc3)C(=O)O)nn2)c(OC)c1OC]

The title compound was prepared in analogy to Example 82 from (S)-ethyl 4-phenyl-2-(1-(2-(2,3,4-trimethoxybenzamido)ethyl)-1H-1,2,3-triazole-4-carboxamido)butanoate, product of Example 80. White solid. LC/HR-MS: (M−H)⁻=510.1996.

### Example 84

### (S)-2-({1-[2-(2-Fluoro-4-trifluoromethyl-benzoylamino)-ethyl]-1H-[1,2,3]triazole-4-carbonyl}-amino)-3-(1H-indol-2-yl)-propionic acid methyl ester

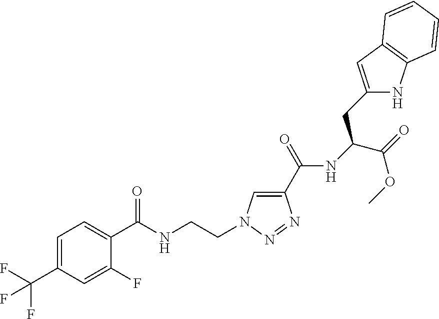 [SMILES: COC(=O)[C@H](Cc1cc2ccccc2[nH]1)NC(=O)c1cn(CCNC(=O)c2ccc(C(F)(F)F)cc2F)nn1]

The title compound was prepared in analogy to Example 1 from 1-(2-(2-fluoro-4-(trifluoromethyl)benzamido)ethyl)-1H-1,2,3-triazole-4-carboxylic acid, product of Example 76 step 2, and L-tryptophan methyl ester, hydrochloride salt. White solid. LC/HR-MS: (M+H)⁺=547.1712.

### Example 85

### 1-[3-(2-Methoxy-4-trifluoromethyl-benzoylamino)-propyl]-1H-[1,2,3]triazole-4-carboxylic acid [cyano-(4-ethyl-phenyl)-methyl]-amide

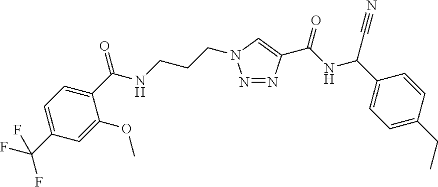 [SMILES: CCc1ccc(C(C#N)NC(=O)c2cn(CCCNC(=O)c3ccc(C(F)(F)F)cc3OC)nn2)cc1]

Step 1

In a 25 mL round-bottomed flask, tert-butyl 3-bromopropylcarbamate (1 g, 4.2 mmol) was combined with N,N-dimethylformamide (12 ml). Sodium azide (300 mg, 4.62 mmol) was added and the reaction was stirred at 80° C. for 2 days. The reaction mixture was diluted with diethyl ether, washed with brine, dried over sodium sulfate and concentrated in vacuo to afford (3-azido-propyl)-carbamic acid tert-butyl ester (736 mg, 88%) as a yellow liquid. This liquid was used in the next step without further purification.

Step 2

In a 25 mL round-bottomed flask, (3-azido-propyl)-carbamic acid tert-butyl ester (736 mg, 3.66 mmol), methyl propiolate (615 mg, 611 μl, 7.31 mmol), copper (II) sulfate pentahydrate (731 μl, 731 μmol) and L(+)Ascorbic acid sodium salt (2.93 ml, 2.93 mmol) were combined with tert-butanol (8 ml) to give a yellow suspension. The reaction mixture was stirred for 2 days at room temperature. The reaction mixture was poured into water and extracted with ethyl acetate. The organic layers were combined, washed with brine and dried over sodium sulfate. The volatiles were removed in vacuo. The residue was purified by chromatography over silica gel (silica gel 70 g, dichloromethane/methanol 100:0 to 85:25). One fraction was isolated and dried in vacuo, affording 874 mg (84%) of 1-(3-tert-butoxycarbonylamino-propyl)-1H-[1,2,3]triazole-4-carboxylic acid methyl ester as a white solid. MS+(m/z): 285.5 (M+H)⁺.

Step 3

In a 25 mL round-bottomed flask, 1-(3-tert-butoxycarbonylamino-propyl)-1H-[1,2,3]triazole-4-carboxylic acid methyl ester (874 mg, 3.07 mmol) was combined with a solution of hydrochloric acid in dioxane (4M, 7.69 ml, 30.7 mmol) to give a yellow suspension. The solution was stirred at room temperature overnight and the volatiles removed in vacuo to afford 1-(3-amino-propyl)-1H-[1,2,3]triazole-4-carboxylic acid methyl ester, hydrochloric acid salt (747 mg; quant.) as a white solid. This solid was used in the next step without further purification. MS−(m/z): 183.5 (M−H)⁻.

Step 4

2-Methoxy-4-trifluoromethyl-carboxylic acid (824 mg, 3.72 mmol) was combined with N,N-dimethylacetamide (30 ml) to give a colorless solution. Diisopropylethylamine (1.31 g, 1.77 ml, 10.2 mmol) was added and the reaction mixture cooled to 0° C. 2-(1H-7-Azabenzotriazol-1-yl)-1,1,3,3-tetramethyl uronium hexafluorophosphate methanaminium (1.42 g, 3.72 mmol) was added and the mixture was stirred in an ice bath for 1 hour. 1-(3-Amino-propyl)-1H-[1,2,3]triazole-4-carboxylic acid methyl ester, hydrochloric acid salt (747 mg, 3.39 mmol) was added and the reaction mixture was stirred at room temperature overnight. The reaction mixture was partitioned between aqueous hydrochloric acid (1M) and ethyl acetate. The aqueous layer was extracted with ethyl acetate. The organic layers were combined, washed with a saturated aqueous solution of sodium hydrogencarbonate, water and brine then dried over sodium sulfate. The volatiles were removed in vacuo and the residue was purified by chromatography over silica gel (silica gel 70 g, heptane/ethyl acetate 70:30 to 0:100). One fraction was isolated and dried in vacuo, affording 940 mg (72%) of 1-[3-(2-methoxy-4-trifluoromethyl-benzoylamino)-propyl]-1H-[1,2,3]triazole-4-carboxylic acid methyl ester as a white solid. MS+(m/z): 387.5 (M+H)⁺.

Step 5

1-[3-(2-Methoxy-4-trifluoromethyl-benzoylamino)-propyl]-1H-[1,2,3]triazole-4-carboxylic acid methyl ester (940 mg, 2.43 mmol) was combined with methyl alcohol (20 ml) to give a light yellow solution. An aqueous solution of sodium hydroxide (1M, 9.73 ml, 9.73 mmol) was added and the reaction mixture was stirred at room temperature overnight. The solvent was removed in vacuo and the residue was diluted with ethyl acetate. The organic layer was washed with aqueous hydrochloric acid (1 M). The aqueous layer was extracted with dichloromethane. The combined organic layers were dried in vacuo affording 1-[3-(2-methoxy-4-trifluoromethyl-benzoylamino)-propyl]-1H-[1,2,3]triazole-4-carboxylic acid (172 mg, 19%) as a an off-white solid. MS−(m/z): 371.5 (M−H)⁻.

Step 6

In a 10 mL round-bottomed flask, 1-[3-(2-methoxy-4-trifluoromethyl-benzoylamino)-propyl]-1H-[1,2,3]triazole-4-carboxylic acid (75 mg, 201 μmol), 2-(1H-7-azabenzotriazol-1-yl)-1,1,3,3-tetramethyl uronium hexafluorophosphate methanaminium (115 mg, 302 μmol) and diisopropylethyl amine (104 mg, 141 μl, 806 μmol) were combined with N,N-dimethylacetamide (3 ml) at 0° C. to give a yellow solution. The solution was stirred at 0° C. for 45 minutes then 2-amino-2-(4-ethylphenyl)acetonitrile hydrochloride (41.6 mg, 212 μmol), product of Intermediate 3, step 1, was added. The reaction mixture was allowed to warm to room temperature and stirred overnight. The reaction mixture was poured into water and extracted with dichloromethane. The organic layers were combined, washed with water and brine then dried over sodium sulfate. The volatiles were removed in vacuo and the residue was purified by chromatography over silica gel (silica gel 10 g, dichloromethane/methanol 99:1 to 85:25). One fraction was isolated and dried in vacuo, affording 68 mg (66%) of the title compound as an off-white solid. MS+(m/z): 515.5 (M+H)⁺.

### Example 86 and 87

### 1-[2-(2,3,4-Trimethoxy-thiobenzoylamino)-ethyl]-1H-[1,2,3]triazole-4-carboxylic acid [cyano-(4-ethyl-phenyl)-methyl]-amide

### and

### N-[2-(4-{[Cyano-(4-ethyl-phenyl)-methyl]-carbamoyl}-[1,2,3]triazol-1-yl)-ethyl]-2,3,4-trimethoxy-thiobenzimidic acid methyl ester

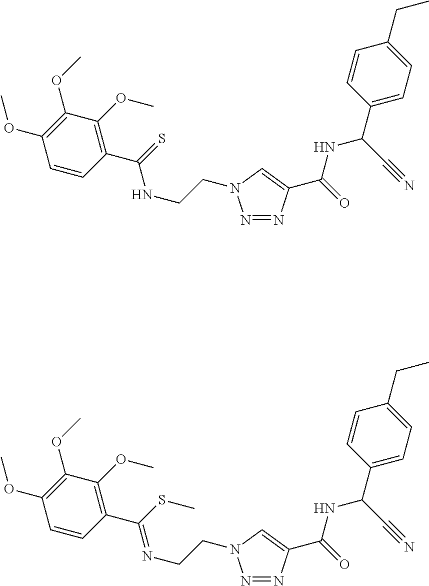

Step 1

In a 100 mL round-bottomed flask, 2,3,4-trimethoxybenzoic acid (617 mg, 2.85 mmol) and 2,4-bis(methylthio)-1,3,2,4-dithiadiphosphatane-2,4-disulfide (500 mg, 1.58 mmol) were combined with 1,2,4-dichlorobenzene (4 ml) to give a yellow suspension. The reaction mixture was heated to 130° C. and stirred for 15 min, then cooled down and concentrated in vacuo. The residue was purified by chromatography over silica gel (50 g, heptane/ethyl acetate 98:2 to 9:1). One fraction was isolated and dried in vacuo, affording 195 mg (48%) of 2,3,4-trimethoxy-thiobenzoic acid S-methyl ester as a red oil.

Step 2

In a 10 mL round-bottomed flask, 2,3,4-trimethoxy-thiobenzoic acid S-methyl ester (65 mg, 252 μmol) and 1-(2-amino-ethyl)-1H-[1,2,3]triazole-4-carboxylic acid [cyano-(4-ethyl-phenyl)-methyl]-amide (75.1 mg, 252 μmol), Intermediate 3, were combined with dichloromethane (2 ml) to give a red suspension. The reaction mixture was stirred at 22° C. for 24 h under inert atmosphere. The crude reaction mixture was concentrated in vacuo. The residue was purified by chromatography over silica gel (20 g, heptane/ethyl acetate 9:1 to 1:1). Two fractions were isolated, dried in vacuo and further purified using HLPC to afford 2 fractions. Fraction 1: 1-[2-(2,3,4-trimethoxy-thiobenzoylamino)-ethyl]-1H-[1,2,3]triazole-4-carboxylic acid [cyano-(4-ethyl-phenyl)-methyl]-amide, 12 mg (9%), yellow solid. MS+(m/z): 509.5 (M+H)⁺. Fraction 2: N-[2-(4-{[cyano-(4-ethyl-phenyl)-methyl]-carbamoyl}-[1,2,3]triazol-1-yl)-ethyl]-2,3,4-trimethoxy-thiobenzimidic acid methyl ester, 28 mg (21%), white solid. MS+(m/z): 523.5 (M+H)⁺.

### Example 88

### 1-[2-(2,3,4-Trimethoxy-benzoylamino)-ethyl]-1H-[1,2,3]triazole-4-carboxylic acid ((S)-1-benzyl-2-benzylcarbamoyl-2-oxo-ethyl)-amide

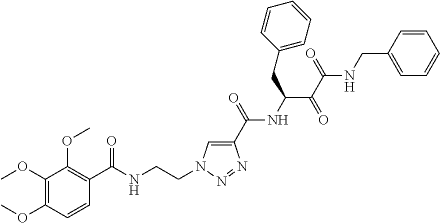 [SMILES: COc1ccc(C(=O)NCCn2cc(C(=O)N[C@@H](Cc3ccccc3)C(=O)C(=O)NCc3ccccc3)nn2)c(OC)c1OC]

Step 1

To a solution of (S)-2-tert-butoxycarbonylamino-3-phenyl-propionic acid (25 g, 94.34 mmol) in DMF (250 mL) were added N,O-dimethylhydroxylamine hydrochloride (13.72 g, 141.50 mmol), HATU (37.64 g, 99.05 mmol) and N,N-diisopropylethylamine (50.70 mL, 283.01 mmol) under nitrogen atmosphere at room temperature. The reaction mixture was stirred at room temperature for 16 h then diluted with ethyl acetate (1000 mL) and washed with water (5×250 mL). The organic layer was dried and concentrated under reduced pressure. The crude residue was purified by CombiFlash column chromatography using 20% EtOAc in hexane to afford 27.5 g (94%) of (S)-1-(methoxy-methyl-carbamoyl)-2-phenyl-ethyl]-carbamic acid tert-butyl ester as colorless oil. LC/MS: (M+H)⁺=309.0.

Step 2

To a stirred solution of (S)-1-(methoxy-methyl-carbamoyl)-2-phenyl-ethyl]-carbamic acid tert-butyl ester (15 g, 48.70 mmol) in THF (180 mL) at 0° C. was added LiAlH₄ (1.0 M in THF, 57 mL, 57 mmol). The reaction mixture was stirred at 0° C. for 1 h then carefully quenched by portionwise addition of sodium sulfate decahydrate until gas evolution ceased. EtOAc was added and the reaction mixture was stirred vigorously at room temperature for 30 min and then filtered. The filtrate was dried and concentrated under reduced pressure to afford 11.0 g (91%) of ((S)-1-benzyl-2-oxo-ethyl)-carbamic acid tert-butyl ester as white solid which was used without further purification.

Step 3

To a solution of ((S)-1-benzyl-2-oxo-ethyl)-carbamic acid tert-butyl ester (7.0 g, 28.1 mmol) in DCM (80 mL) was added acetone cyanohydrin (7.16 g, 84.3 mmol) and triethylamine (2.36 mL, 16.86 mmol). The reaction was stirred at room temperature for 3 h then water was added and the organics were removed under reduced pressure. The aqueous residue was extracted with ethyl acetate and washed twice with water. The organic layer was dried and concentrated under reduced pressure. The crude residue was purified by CombiFlash column chromatography using 20% EtOAc in hexane as mobile phase to obtain 5.0 g (58%) of ((S)-1-benzyl-2-cyano-2-hydroxy-ethyl)-carbamic acid tert-butyl ester as yellow oil. LC/MS: (M+H)⁺=277.4.

Step 4

A solution of ((S)-1-benzyl-2-cyano-2-hydroxy-ethyl)-carbamic acid tert-butyl ester (5.0 g, 18.11 mmol) in 6M HCl (90 mL) was heated at 100° C. for 16 h then cooled to room temperature and concentrated under vacuum to afford4.0 g (95%) of (S)-3-amino-2-hydroxy-4-phenyl-butyric acid hydrochloride as off yellow solid which was used without further purification. LC/MS: (M+H)⁺=196.2.

Step 5

To a solution of (S)-3-amino-2-hydroxy-4-phenyl-butyric acid hydrochloride (18.0 g, 77.9 mmol) in 1,4-dioxane (150 ml) and water (150 mL) were added sodium bicarbonate (65.45 g 779 mmol) and di-tert-butyl dicarbonate (25.48 g, 116.9 mmol). The mixture was stirred vigorously at room temperature for 16 h. The organic phase was removed under reduced pressure. The remaining heterogeneous aqueous layer was diluted with water (200 mL) and extracted with Et₂O (2×200 mL, discarded). Then the aqueous layer was brought to pH=3 by addition of aqueous 2 M HCl and extracted with EtOAc (3×400 mL). The combined extracts were dried and concentrated under reduced pressure to afford 18.0 g (78%) of (S)-3-tert-butoxycarbonylamino-2-hydroxy-4-phenyl-butyric acid as off white solid. LC/MS: (M+H)⁺=296.6.

Step 6

To a stirred solution of (S)-3-tert-butoxycarbonylamino-2-hydroxy-4-phenyl-butyric acid (10.0 g, 33.89 mmol) in DMF (150 mL) were added benzylamine (4.35 g, 40.67 mmol), HATU (14.16 g, 37.28 mmol) and N,N-diisopropylethylamine (6.56 g, 50.84 mmol). The reaction mixture was stirred under nitrogen atmosphere at room temperature for 3 h then diluted with ethyl acetate (800 mL) and washed with ice cold water (2×950 mL). The organic layer was dried over sodium sulfate and concentrated under reduced pressure. The crude residue was purified by CombiFlash column chromatography using 30% EtOAc in hexane to provide 7.3 g (56%) of (S)-1-benzyl-2-benzylcarbamoyl-2-hydroxy-ethyl)-carbamic acid tert-butyl ester as white solid. LC/MS: (M+H)⁺=385.2.

Step 7

In a 10 ml round-bottomed flask, (S)-1-benzyl-2-benzylcarbamoyl-2-hydroxy-ethyl)-carbamic acid tert-butyl ester (120 mg, 0.31 mmol) was dissolved in 3 ml dichloromethane. Trifluoroacetic acid (977 mg, 0.66 ml, 8.57 mmol) was added slowly. The reaction mixture was stirred at room temperature for 3 h then the solvent was evaporated and the residue placed under high vacuum for ˜30 min. The residue and 1-[2-(2,3,4-trimethoxy-benzoylamino)-ethyl]-1H-[1,2,3]triazole-4-carboxylic acid (Intermediate 2, 100 mg, 0.285 mmol) were dissolved in 2 ml DMF and the light brown solution was cooled to 0° C. N,N-Diisopropylethylamine (222 mg, 0.30 ml, 1.72 mmol) was added dropwise at 0° C. followed by HATU (119 mg, 0.31 mmol). After the addition was complete, the ice bath was removed and the reaction mixture was stirred at room temperature overnight. The reaction mixture was diluted with water. The resulting suspension was filtered, rinsed with water and a little petroleum ether and dried under high vacuum to afford 156 mg (89%) of 1-[2-(2,3,4-trimethoxy-benzoylamino)-ethyl]-1H-[1,2,3]triazole-4-carboxylic acid ((S)-1-benzyl-2-benzylcarbamoyl-2-hydroxy-ethyl)-amide as an off-white solid and a mixture of diastereomers. LC/MS: (M−H)⁻=615.2.

Step 8

In a 50 ml round-bottomed flask, 1-[2-(2,3,4-trimethoxy-benzoylamino)-ethyl]-1H-[1,2,3]triazole-4-carboxylic acid ((S)-1-benzyl-2-benzylcarbamoyl-2-hydroxy-ethyl)-amide (154 mg, 0.25 mmol) was partially dissolved in 10 ml dichloromethane and Dess-Martin periodinane (159 mg, 0.375 mmol) was added. The reaction mixture was stirred at room temperature for 1.5 h then quenched with 3.5 ml saturated NaHCO₃-solution and 3.5 ml 10% Na₂S₂O₃-solution. The biphasic mixture was stirred vigorously for 1 h at room temperature then extracted with dichloromethane. The organic layer was washed with saturated NaHCO₃-solution. The combined aqueous layers were extracted twice with dichloromethane. The organic layers were combined, dried over sodium sulfate, filtered and concentrated. The residue was absorbed on silica gel and chromatographed over 12 g silica gel with Me0H/dichloromethane (gradient: 0-5% MeOH). All fractions containing product were combined and concentrated. The residue was triturated with dichloromethane/diethyl ether/hexanes to afford 64 mg (40%) of 1-[2-(2,3,4-trimethoxy-benzoylamino)-ethyl]-1H-[1,2,3]triazole-4-carboxylic acid ((S)-1-benzyl-2-benzylcarbamoyl-2-oxo-ethyl)-amide as an off-white solid. LC/MS: (M−H)⁻=613.2.

### Example 89

### 5-[2-(2,3,4-Trimethoxy-benzoylamino)-ethyl]-2H-[1,2,4]triazole-3-carboxylic acid [cyano-(4-ethyl-phenyl)-methyl]-amide

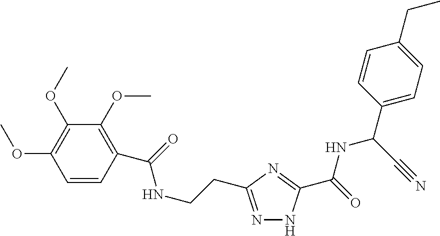 [SMILES: CCc1ccc(C(C#N)NC(=O)c2nc(CCNC(=O)c3ccc(OC)c(OC)c3OC)n[nH]2)cc1]

Step 1

In a 50 mL round-bottomed flask, ethyl 2-amino-2-thioxoacetate (0.6 g, 4.51 mmol) and tert-butyl 3-hydrazinyl-3-oxopropylcarbamate (981 mg, 4.73 mmol) were combined and stirred 1 h at 65° C., affording 1.36 g (quant.) of ethyl 2-amino-2-(2-(3-(tert-butoxycarbonylamino)propanoyl)hydrazono)acetate as yellow gum. This gum was used in the next step without further purification. MS+(m/z): 303.5 (M+H)⁺.

Step 2

In a 10 mL round-bottomed flask, ethyl 2-amino-2-(2-(3-(tert-butoxycarbonylamino)propanoyl)hydrazono)acetate (1.36 g, 4.5 mmol) was combined with diglyme (3 ml) to give a yellow solution. The reaction mixture was stirred 1 h at 200° C.

The reaction mixture was partitioned between water and ethyl acetate. The aqueous layer was extracted with ethyl acetate. Combined organic layers were washed with brine then dried over sodium sulfate and concentrated in vacuo. The residue was purified by chromatography over silica gel (70 g, heptane/ethyl acetate 1:1 to dichloromethane/methanol 98:2). One fraction was isolated and dried in vacuo, affording 350 mg (27%) of ethyl 3-(2-(tert-butoxycarbonylamino)ethyl)-1H-1,2,4-triazole-5-carboxylate as yellow foamy solid.

Step 3

In a 10 mL round-bottomed flask, ethyl 3-(2-(tert-butoxycarbonylamino)ethyl)-1H-1,2,4-triazole-5-carboxylate (340 mg, 1.2 mmol) was combined with formic acid (1.65 g, 1.38 ml, 35.9 mmol). The reaction mixture was stirred 3h at room temperature then concentrated in vacuo affording 653 mg (50%) of ethyl 3-(2-aminoethyl)-1H-1,2,4-triazole-5-carboxylate formate as a white solid. MS+(m/z): 185.3 (M+H)⁺.

Step 4

In a round-bottomed flask, 2,3,4-trimethoxybenzoic acid (359 mg, 1.69 mmol), 2-(1H-7-azabenzotriazol-1-yl)-1,1,3,3-tetramethyl uronium hexafluorophosphate methanaminium (1.07 g, 2.82 mmol) and diisopropylethylamine (500 ul) were combined with N,N-dimethylacetamide (5 ml) at 0° C. to give a light yellow solution. The reaction mixture was stirred at 0° C. for 1 h then ethyl 3-(2-aminoethyl)-1H-1,2,4-triazole-5-carboxylate formate (650 mg, 1.41 mmol) and diisopropylethylamine (1 ml) were added. The reaction mixture was allowed to warm up to room temperature and stirred for 20 h. The reaction mixture was partitioned between a saturated aqueous solution of sodium hydrogencarbonate and dichloromethane. The aqueous layer was extracted with dichloromethane. The combined organic layers were washed with water and brine then dried over sodium sulfate and concentrated in vacuo. The residue was purified by chromatography over silica gel (50 g, dichloromethane/methanol 98:2 to 19:1). One fraction was isolated and dried in vacuo, affording 190 mg (36%) of ethyl 3-(2-(2,3,4-trimethoxybenzamido)ethyl)-1H-1,2,4-triazole-5-carboxylate as light brown solid. MS+(m/z): 379.5 (M+H)⁺.

Step 5

In a 10 mL round-bottomed flask, ethyl 3-(2-(2,3,4-trimethoxybenzamido)ethyl)-1H-1,2,4-triazole-5-carboxylate (90 mg, 238 μmol) was combined with methanol (1.5 ml) to give a light brown solution. Aqueous sodium hydroxide solution (1N, 714 μl, 714 μmol) was added. The reaction mixture was stirred at room temperature for 24 h then concentrated in vacuo, affording 114 mg (quant.) of sodium 3-(2-(2,3,4-trimethoxybenzamido)ethyl)-1H-1,2,4-triazole-5-carboxylate as a light brown solid. The solid was used in the next step without further purification. MS+(m/z): 351.5 (M+H)⁺.

Step 6

In a 25 mL three-necked flask N,N-dimethylformamide (0.2 ml) in acetonitrile (1.5 ml) was stirred at −20° C. Then oxalyl chloride (45.9 mg, 31.1 μl, 355 μmol) was slowly added. After 15 min, sodium 3-(2-(2,3,4-trimethoxybenzamido)ethyl)-1H-1,2,4-triazole-5-carboxylate (110 mg, 236 μmol) was added. After stirring further 20 min at −20° C., a solution of 2-amino-2-(4-ethylphenyl)acetonitrile hydrochloride (Example 85, Step 6, 48.8 mg, 248 μmol) and pyridine (122 mg, 124 μl, 1.54 mmol) in 1.5 ml of acetonitrile was added dropwise. The reaction mixture was allowed to warm up to room temperature for 30 min and then stirred at 80° C. for 1 h.

Aqueous hydrochloric acid (1N, 2 ml) was added and the reaction mixture was concentrated in vacuo. The residue was partitioned between aqueous hydrochloric acid (0.1 M) and dichloromethane. The aqueous layer was extracted with dichloromethane. The combined organic layers were dried over sodium sulfate and dried in vacuo. The residue was purified by chromatography over silica gel (20 g, dichloromethane/methanol 98:2 to 19:1). One fraction was isolated and dried in vacuo, affording 7 mg (48%) of the title compound as a yellow oil. MS+(m/z): 493.6 (M+H)⁺.

### Example 90

### 5-[2-(2-Methoxy-4-trifluoromethyl-benzoylamino)-ethyl]-2H-[1,2,4]triazole-3-carboxylic acid [cyano-(4-ethyl-phenyl)-methyl]-amide

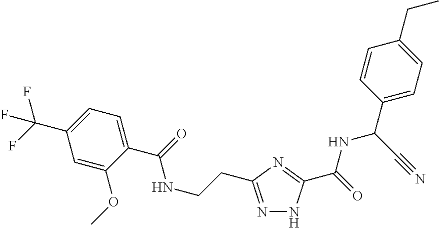 [SMILES: CCc1ccc(C(C#N)NC(=O)c2nc(CCNC(=O)c3ccc(C(F)(F)F)cc3OC)n[nH]2)cc1]

Step 1

In a 10 mL round-bottomed flask, ethyl 3-(2-(tert-butoxycarbonylamino)ethyl)-1H-1,2,4-triazole-5-carboxylate (cf. Example 89, step 2, 100 mg, 352 μmol) was combined with methanol (2 ml). An aqueous solution of sodium hydroxide (3M, 352 μl, 1.06 mmol) was added and the reaction mixture was stirred 16 h at room temperature. The reaction mixture was concentrated in vacuo, affording 180 mg (quant.) of sodium 3-(2-(tert-butoxycarbonylamino)ethyl)-1H-1,2,4-triazole-5-carboxylate as a light brown solid.

Step 2

In a 10 mL round-bottomed flask, sodium 3-(2-(tert-butoxycarbonylamino)ethyl)-1H-1,2,4-triazole-5-carboxylate (50 mg, 180 μmol) and 2-amino-2-(4-ethylphenyl)acetonitrile hydrochloride (Intermediate 3, step 1, 35.3 mg, 180 μmol) followed by O-(benzotriazol-1-yl)-N,N,N′,N′-tetramethyluronium tetrafluoroborate (63.5 mg, 198 μmol) and diisopropylethylamine (116 mg, 157 μl, 899 μmol) were combined in dichloromethane (3 ml) to give a yellow suspension. The reaction mixture was stirred at 22° C. for 16 h. The reaction mixture was partitioned between water and dichloromethane. The aqueous layer was extracted with dichloromethane. The combined organic layers were washed with water and brine then dried over sodium sulfate and concentrated in vacuo. The residue was purified by chromatography over silica gel (10 g, heptane/dichloromethane/methanol 1:1:0 to 0:98:2). One fraction was isolated and dried in vacuo, affording 40 mg (45%) of tert-butyl 2-(5-(cyano(4-ethylphenyl)methylcarbamoyl)-1H-1,2,4-triazol-3-yl)ethylcarbamate as a yellow oil. MS−(m/z): 397.6 (M−H)⁻.

Step 3

In a 10 mL round-bottomed flask, tert-butyl 2-(5-(cyano(4-ethylphenyl)methylcarbamoyl)-1H-1,2,4-triazol-3-yl)ethylcarbamate (40 mg, 100 μmol) was combined with formic acid (924 mg, 770 μl, 20.1 mmol). The reaction mixture was stirred 16 h at room temperature then concentrated in vacuo. The reaction mixture was partitioned between a aqueous solution of sodium carbonate (5%) and dichloromethane. The aqueous layer was extracted with dichloromethane. The combined organic layers were dried over sodium sulfate and concentrated in vacuo, affording 27 mg (90%) of 3-(2-aminoethyl)-N-(cyano(4-ethylphenyl)methyl)-1H-1,2,4-triazole-5-carboxamide as a light brown oil. MS+(m/z): 299.5 (M+H)⁺.

Step 4

In a round-bottomed flask, 2-methoxy-4-(trifluoromethyl)benzoic acid (23.9 mg, 109 μtmol), 2-(1H-7-azabenzotriazol-1-yl)-1,1,3,3-tetramethyl uronium hexafluorophosphate methanaminium (51.6 mg, 136 μmol) and diisopropylethylamine (46.8 mg, 63.2 μl, 362 μmol) were combined with N,N-dimethylacetamide (1 ml) at 0° C. to give a light yellow solution. The reaction mixture was stirred at 0° C. for 1 h then 3-(2-aminoethyl)-N-(cyano(4-ethylphenyl)methyl)-1H-1,2,4-triazole-5-carboxamide (27 mg, 90.5 μmol) was added. The reaction mixture was allowed to warm up to room temperature and stirred for 16 h. The reaction mixture was partitioned between a saturated aqueous solution of sodium hydrogencarbonate and dichloromethane. The aqueous layer was extracted with dichloromethane. The combined organic layers were washed with water and brine then dried over sodium sulfate and concentrated in vacuo. The residue was purified by chromatography over silica gel (20 g, dichloromethane/methanol 99:1 to 98:2). One fraction was isolated and dried in vacuo, affording 7 mg (13%) of the title compound as light brown solid. MS+(m/z): 501.5 (M+H)⁺.

### Example 91

### 5-[2-(2-Fluoro-4-trifluoromethyl-benzoylamino)-ethyl]-2H-[1,2,4]triazole-3-carboxylic acid [cyano-(4-ethyl-phenyl)-methyl]-amide

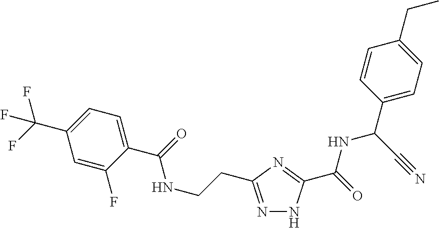 [SMILES: CCc1ccc(C(C#N)NC(=O)c2nc(CCNC(=O)c3ccc(C(F)(F)F)cc3F)n[nH]2)cc1]

The title compound was prepared from 3-(2-aminoethyl)-N-(cyano(4-ethylphenyl)methyl)-1H-1,2,4-triazole-5-carboxamide (Example 90, step 3), and 2-fluoro-4-(trifluoromethyl)benzoic acid in analogy to Example 90, step 4. Off-white solid. MS+(m/z): 489.6 (M+H)⁺.

### Example 92

### 1-[2-(2-Methoxy-4-trifluoromethyl-benzoylamino)-ethyl]-1H-imidazole-4-carboxylic acid [cyano-(4-ethyl-phenyl)-methyl]-amide

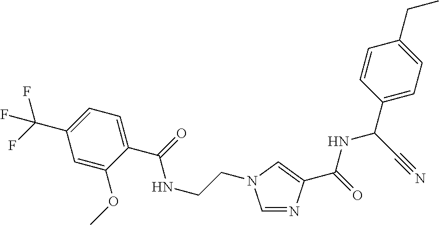 [SMILES: CCc1ccc(C(C#N)NC(=O)c2cn(CCNC(=O)c3ccc(C(F)(F)F)cc3OC)cn2)cc1]

Step 1

In a 100 mL round-bottomed flask, methyl 1H-imidazole-4-carboxylate (1.07 g, 8.48 mmol) and a suspension of sodium hydride (60% in oil, 356 mg, 8.91 mmol) were combined with N,N-dimethylformamide (15 ml). The reaction mixture was stirred 2 h at 90° C. then cooled down to room temperature and 2-(2-bromoethyl)isoindoline-1,3-dione (2.38 g, 8.91 mmol) was added. The reaction mixture was stirred 16 h at 90° C. then concentrated in vacuo and partitioned between water and dichloromethane. The aqueous layer was extracted with dichloromethane. The combined organic layers were washed with water and brine then dried over sodium sulfate and concentrated in vacuo. The residue was purified by chromatography over silica gel (100 g, heptane/ ethyl acetate 1:1 to 1:5). One fraction was isolated and dried in vacuo, affording 927 mg (37%) of methyl 1-(2-(1,3-dioxoisoindolin-2-yl)ethyl)-1H-imidazole-4-carboxylate as a white solid, along with its isomer (373 mg, 15%, discarded). MS+(m/z): 300.5 (M+H)⁺.

Step 2

In a 25 mL round-bottomed flask, methyl 1-(2-(1,3-dioxoisoindolin-2-yl)ethyl)-1H-imidazole-4-carboxylate (300 mg, 1.00 mmol) was combined with ethanol (5 ml) to give a white suspension. A solution of hydrazine hydrate in water (24-26% , 502 mg, 501 μl, 2.51 mmol) was added and the reaction mixture was stirred 2 h at 85° C. The reaction mixture was concentrated in vacuo. The residue was partitioned between water and dichloromethane. The pH of the aqueous layer was adjusted to ca. 5 by adding an aqueous solution of sodium carbonate (5%). The aqueous layer was then extracted with dichloromethane. The combined organic layers were dried over sodium sulfate then concentrated in vacuo, affording 127 mg (75%) of methyl 1-(2-aminoethyl)-1H-imidazole-4-carboxylate as a light yellow powder. MS+(m/z): 170.5 (M+H)⁺.

Step 3

In a round-bottomed flask, 2-methoxy-4-(trifluoromethyl)benzoic acid (157 mg, 715 μmol), 2-(1H-7-azabenzotriazol-1-yl)-1,1,3,3-tetramethyl uronium hexafluorophosphate methanaminium (371 mg, 975 μmol) and diisopropylethylamine (336 mg, 454 μl, 2.6 mmol) were combined with N,N-dimethylacetamide (5 ml) at 0° C. to give a yellow solution. The reaction mixture was stirred at 0° C. for 1 h then methyl 1-(2-aminoethyl)-1H-imidazole-4-carboxylate (110 mg, 650 μmol) was added. The reaction mixture was allowed to warm up to room temperature and stirred for 16 h. The reaction mixture was partitioned between a saturated aqueous solution of sodium hydrogencarbonate and dichloromethane. The aqueous layer was extracted with dichloromethane. Combined organic layers were washed with water and brine then dried over sodium sulfate and concentrated in vacuo. The residue was purified by chromatography over silica gel (20 g, dichloromethane/methanol 99:1 to 98:2). One fraction was isolated and dried in vacuo, affording 200 mg (83%) of methyl 1-(2-(2-methoxy-4-(trifluoromethyl)benzamido)ethyl)-1H-imidazole-4-carboxylate as a light brown solid. MS+(m/z): 372.6 (M+H)⁺.

Step 4

In a 10 mL round-bottomed flask, methyl 1-(2-(2-methoxy-4-(trifluoromethyl)benzamido)ethyl)-1H-imidazole-4-carboxylate (180 mg, 485 μmol) was combined with methanol (3 ml) to give a light yellow solution. An aqueous solution of sodium hydroxide (3M, 485 μl, 1.45 mmol) was added. The reaction mixture was stirred at room temperature for 2 days and concentrated in vacuo. The reaction mixture was partitioned between an aqueous solution of hydrochloric acid (1M) and dichloromethane. The aqueous layer was extracted with dichloromethane. The combined organic layers were dried over sodium sulfate, filtered an concentrated in vacuo, affording 160 mg (92%) of 1-(2-(2-methoxy-4-(trifluoromethyl)benzamido)ethyl)-1H-imidazole-4-carboxylic acid as an off-white solid. MS+(m/z): 358.5 (M+H)⁺.

Step 5

In a round-bottomed flask, 1-(2-(2-methoxy-4-(trifluoromethyl)benzamido)ethyl)-1H-imidazole-4-carboxylic acid (78 mg, 218 μmol,), 2-(1H-7-azabenzotriazol-1-yl)-1,1,3,3-tetramethyluronium hexafluorophosphate methanaminium (125 mg, 327 μmol) and diisopropylethylamine (113 mg, 153 μl, 873 μmol) were combined with N,N-dimethylacetamide (5 ml) at 0° C. to give a light yellow solution. The reaction mixture was stirred at 0° C. for 1 h then 2-amino-2-(4-ethylphenyl)acetonitrile hydrochloride (Intermediate 3, step 1, 47.2 mg, 240 μmol) was added. The reaction mixture was allowed to warm up to room temperature and stirred for 16 h. The reaction mixture was partitioned between a saturated aqueous solution of sodium hydrogencarbonate and dichloromethane. The aqueous layer was extracted with dichloromethane. The combined organic layers were washed with water and brine then dried over sodium sulfate and concentrated in vacuo. The residue was purified by chromatography over silica gel (20 g, dichloromethane/methanol 99:1 to 98:2). One fraction was isolated and dried in vacuo, affording 46 mg (42%) of the title compound as yellow solid. MS+(m/z): 500.6 (M+H)⁺.

### Example 93

### 1-[2-(2-Methoxy-4-trifluoromethyl-benzoylamino)-ethyl]-1H-imidazole-4-carboxylic acid [(4-bromo-phenyl)-cyano-methyl]-amide

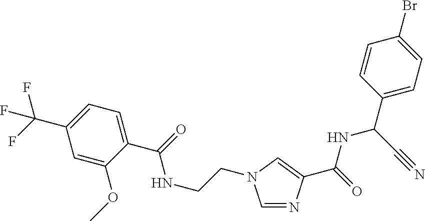 [SMILES: COc1cc(C(F)(F)F)ccc1C(=O)NCCn1cnc(C(=O)NC(C#N)c2ccc(Br)cc2)c1]

The title compound was prepared from 1-(2-(2-methoxy-4-(trifluoromethyl)benzamido)ethyl)-1H-imidazole-4-carboxylic acid (Example 92, step 4), and 2-amino-2-(4-bromophenyl)acetonitrile hydrochloride (Example 54, step 1) in analogy to Example 92, step 5. Light brown solid. MS+(m/z): 550.5 (M+H)⁺.

### Example 94

### 1-[2-(2-Fluoro-4-trifluoromethyl-benzoylamino)-ethyl]-1H-imidazole-4-carboxylic acid [cyano-(4-ethyl-phenyl)-methyl]-amide

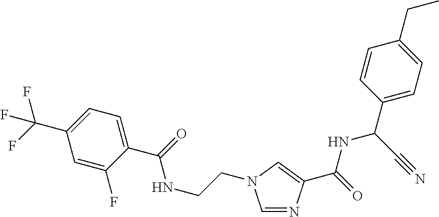 [SMILES: CCc1ccc(C(C#N)NC(=O)c2cn(CCNC(=O)c3ccc(C(F)(F)F)cc3F)cn2)cc1]

Step 1

Methyl 1-(2-(2-fluoro-4-(trifluoromethyl)benzamido)ethyl)-1H-imidazole-4-carboxylate was prepared from 2-fluoro-4-(trifluoromethyl)benzoic acid, and methyl 1-(2-aminoethyl)-1H-imidazole-4-carboxylate (Example 92, step 2) in analogy to Example 92, step 3. White solid. MS+(m/z): 360.5 (M+H)⁺.

Step 2

1-(2-(2-Fluoro-4-(trifluoromethyl)benzamido)ethyl)-1H-imidazole-4-carboxylic acid was prepared from methyl 1-(2-(2-fluoro-4-(trifluoromethyl)benzamido)ethyl)-1H-imidazole-4-carboxylate, in analogy to Example 92, step 4. Off-white solid. MS+(m/z): 346.5 (M+H)⁺.

Step 3

The title compound was prepared from 1-(2-(2-fluoro-4-(trifluoromethyl)benzamido)ethyl)-1H-imidazole-4-carboxylic acid, and 2-amino-2-(4-ethylphenyl)acetonitrile hydrochloride (Intermediate 3, step 1) in analogy to Example 92, step 5. White solid. MS+(m/z): 488.7 (M+H)⁺.

### Example 95

### Assay Protocols and Results

Cell-Based Proteasome Activity/Selectivity Assay

The Cell-Based Proteasome subunit activity/selectivity assay was a panel of 5 fluorogenic assays that independently measured the activity of β5c or β 5i (chymotrypsin-like activity), β 2c/2i (trypsin-like), and β 1c or β 1i (caspase-like) protease activity associated with the proteasome complex in cultured cells. Specifically, the following substrates were used for respective subunit activities: β 1i (PAL)₂Rh110, β 1c: (LLE)₂ Rh110, β 2c/2i: (KQL)₂Rh110, β 5c: (WLA)₂Rh110, β 5i: (ANW)₂Rh110. The following procedure was followed:

Cell preparation: Plated 25 μl of Ramos cells (2×10⁶/ml in DPBS) into half area plate (PerkinElmer Cat 6005569) to final 5×10⁴cells/well. Added 0.5 μl of 100× 4-fold serial diluted test compounds or DMSO to each well. Highest concentration of compound tested was 20 μM, thus compound serial dilution started from 200 mM. Incubated for 30 minutes at 37° C. Then equilibrated at room temperature for 15 minutes. Added 25 μl of 2× reaction mix consisting of 0.025% digitonin, 20 μM of each substrates and 0.5M sucrose in DPBS. Shaked for one minute @ 700 rpm. Incubated for 120 min at room temperature. Then read the plates with an Envision multilabel plate reader (PerkinElmer) with 500 nm excitation/519 nm emission.

Modified PBMC Proteasome Activity Assay

This cell-based proteasome activity assay was similar to previous Ramos cell-based assay as of the substrates, but using human PBMCs in the context of complete RPMI with 10% FBS as reaction buffer. This assay was designed to assess the level of cellular penetration of test compounds in primary human cells. The following procedure was followed: Fresh isolated PBMC from healthy donor were plated at 1×10⁵cells/well in 100 μl of complete RPMI with 10% FBS in V bottom 96 plates. Added 1 μl of 100× 4-fold serial diluted compounds/well and incubated for 1 hr. The highest compound concentration tested was 20 μM (100× working stock start with 2 mM). Spun down the cells @ 2000rpm for 5 mM. Removed all supernatant. Then resuspended the cells in 25 μl DPBS and transferred the cells to a fresh half-area plate (PerkinElmer Cat 6005569). In the final reaction volume was 50 including 25 μl cell suspension, 0.5 μl 100× inhibitor or DMSO, 25 μl substrate mix containing 0.025% digitonin, 20 uM substrate (Substrate: (PAL)₂Rh110, (LLE)₂ Rh110, (KQL)₂Rh110, (WLA)₂Rh110, or (ANW)₂Rh110)/in 10% FBS and 0.5M sucrose mixture. Shaked for one minute (@ 700 rpm). Incubated for 2 hrs, then read the plates with Envision plate reader using 500 nm excitation/519 nm emission.

PBMC IP-10 Assay

PBMCs were isolated from whole blood as follows: Blood was collected in a sterile environment in heparinized tubes. Blood was diluted with an equal volume PBS/2% FCS and 30 ml of this mixture was added to ACCUSPIN tubes containing 15 ml Histopaque-1077 already centrifuged at 800 g for 30 seconds and warmed up at room temperature. The tubes were then centrifuged at 800 g for 20 minutes at room temperature with no brake. The mononuclear band, just above the polyethylene frit, was removed by Pasteur pipet. These mononuclear cells were washed three times with sterile PBS, counted, and resuspended in RPMI 1640 supplemented with 10% heat inactivated fetal calf serum, 10 mM HEPES, 1 mM sodium pyruvate, penicillin (50 U/ml), streptomycin (50 μg/ml) and glutamine (2 mM) to approximately 1.5×10⁶/ml. Approximately 2×10⁵ cells/well were plated in 96 well tissue culture plates (BD Falcon 353072), and preincubated 60 ml/37° C. with a titration of compounds, in a final concentration of 1% DMSO. Cells were then stimulated with CpG Type A (Invivogen, Cat #tlrl-2216; ODN 2216) at a final concentration of 2.5 μells were incubated overnight, and supernatants were removed. PBMC viability of cells remaining in the well was measured with ATPlite luminescence assay (Perkin-Elmer) per the manufacturer's instructions. Luminescence was measured on the Perkin-Elmer Envision, using the luminescence filter. IP10 level was measured with CXCL10/IP10 AlphaLISA kit (Perkin-Elmer) per the manufacturer's instructions, except halving all volumes. Fluorescence was measured on the Envision Multilabel plate reader, using the AlphaScreen standard settings.

Results:

The results of the above assays for representative compounds of the invention are provided in Table 1 below, wherein the IC50 activity values are in 1 μM:

TABLE 1

| | Ic50:ramos:ac- | Ic50:ramos:rh110- | Ic50:ramos:rh110- | Ic50:ramos:rh110- | Ic50:ramos:rh110- |
|---|---|---|---|---|---|
| Example | (anw)2-r110 | (wla)2 | (kql)2 | (pal)2 | (lle)2 |
| 1 | 0.08 | 20 | 20 | 20 | 20 |
| 2 | 0.15 | 20 | 20 | 20 | 20 |
| 3 | 2.897 | 20 | 20 | 20 | 20 |
| 4 | 4.108 | 20 | 20 | 20 | 20 |
| 5 | 6.492 | 20 | 20 | 20 | 20 |
| 6 | 0.919 | 20 | 20 | 20 | 20 |
| 7 | 1.162 | 20 | 20 | 20 | 20 |
| 8 | 0.423 | 20 | 20 | 20 | 20 |
| 9 | 4.29 | 20 | 20 | 20 | 20 |
| 10 | 0.886 | 20 | 20 | 20 | 20 |
| 11 | 0.106 | 9.975 | 20 | 20 | 20 |
| 12 | 1.264 | 20 | 20 | 20 | 20 |
| 13 | 1.891 | 20 | 20 | 20 | 20 |
| 14 | 0.19 | 20 | 20 | 20 | 20 |
| 15 | 1.496 | 7.758 | 20 | 20 | 19.931 |
| 16 | 1.117 | 20 | 20 | | 20 |
| 17 | 9.934 | 20 | 20 | | 20 |
| 18 | 0.482 | 20 | 20 | | 20 |
| 19 | 0.111 | 12.015 | 20 | | 20 |
| 20 | 0.196 | 20 | 20 | | 20 |
| 21 | 2.878 | 20 | 20 | | 20 |
| 22 | 9.952 | 20 | 20 | 20 | 20 |
| 23 | 0.028 | 16.164 | 20 | 20 | 20 |
| 24 | 0.779 | 20 | 20 | 20 | 20 |
| 25 | 0.072 | 20 | 20 | 20 | 20 |
| 26 | 5.381 | 20 | 20 | 20 | 20 |
| 27 | 4.517 | 20 | 20 | 20 | 20 |
| 28 | 0.933 | 13.27 | 20 | 20 | 20 |
| 29 | 3.053 | 20 | 20 | 20 | 20 |
| 30 | 1.019 | 6.778 | 20 | 20 | 16.97 |
| 31 | 0.134 | 16.071 | 20 | 20 | 20 |
| 32 | 0.682 | 20 | 20 | 20 | 20 |
| 33 | 1.802 | 20 | 20 | 20 | 20 |
| 34 | 0.259 | 20 | 20 | 20 | 20 |
| 35 | 0.491 | 5.488 | 20 | 20 | 20 |
| 36 | 2.056 | 20 | 20 | 20 | 20 |
| 37 | 0.071 | 20 | 20 | 20 | 20 |
| 38 | 0.017 | 20 | 20 | 20 | 20 |
| 39 | 0.517 | 20 | 20 | 20 | |
| 40 | 4.059 | 20 | 20 | 20 | |
| 41 | 0.282 | 20 | 20 | 20 | 20 |
| 42 | 3.151 | 20 | 20 | 20 | 20 |
| 43 | 1.432 | 6.208 | 20 | 20 | 20 |
| 44 | 2.927 | 20 | 20 | 20 | 20 |
| 45 | 1.13 | 20 | 20 | 20 | 20 |
| 46 | 5.937 | 16.14 | 20 | 20 | 20 |
| 47 | 1.486 | 7.668 | 20 | 20 | 20 |
| 48 | 0.201 | 16.256 | 20 | | 20 |
| 49 | 0.338 | 12.5 | 20 | | 18.981 |
| 50 | 0.058 | 20 | 20 | 20 | 20 |
| 51 | 0.604 | 20 | 20 | 20 | 20 |
| 52 | 2.695 | 20 | 20 | 20 | 20 |
| 53 | 0.585 | 20 | 20 | 20 | 20 |
| 54 | 0.101 | 20 | 20 | 20 | 20 |
| 55 | 4.024 | 20 | 20 | 20 | 20 |
| 56 | 5.683 | 8.299 | 20 | 20 | 19.083 |
| 57 | 6.493 | 20 | 20 | 20 | 20 |
| 58 | 0.159 | 20 | 20 | 20 | |
| 59 | 5.667 | 20 | 20 | 20 | 20 |
| 60 | 0.619 | 20 | 20 | 20 | 20 |
| 61 | 1.314 | 20 | 20 | 20 | 17.122 |
| 62 | 0.404 | 20 | 20 | 20 | 20 |
| 63 | 2.492 | 20 | 20 | 20 | 20 |
| 64 | 0.127 | 8.309 | 20 | 10.143 | 9.677 |
| 65 | 0.947 | 20 | 20 | 20 | 17.755 |
| 66 | 0.795 | 20 | 20 | 20 | 20 |
| 67 | 5.492 | 20 | 20 | 20 | 20 |
| 68 | 0.327 | 16.816 | 20 | 20 | 20 |
| 69 | 3.791 | 20 | 20 | 20 | |
| 70 | 0.445 | 10.756 | 20 | 20 | 20 |
| 71 | 5.591 | 20 | 20 | 20 | 20 |
| 72 | 0.524 | 20 | 20 | 20 | 20 |
| 73 | 0.184 | 20 | 20 | 20 | 20 |
| 74 | 1.738 | 20 | 20 | 20 | 20 |
| 75 | 1.388 | 19.944 | 20 | 20 | 20 |
| 76 | 0.05 | 20 | 20 | 20 | 20 |
| 77 | 8.749 | 20 | 20 | 20 | 20 |
| 78 | 0.176 | 14.286 | 20 | 20 | 20 |
| 79 | 0.159 | 3.386 | 20 | 0.976 | 20 |
| 80 | 0.447 | 20 | 20 | 20 | 20 |
| 81 | 0.205 | 20 | 20 | 20 | 20 |
| 82 | 0.163 | 20 | 20 | 20 | 20 |
| 83 | 0.921 | 8.364 | 20 | 20 | 20 |
| 84 | 0.162 | 20 | 20 | 20 | 20 |
| 85 | 0.239 | 20 | 20 | 20 | 20 |
| 86 | 0.274 | 16.255 | 20 | 20 | |
| 87 | 2.467 | 8.553 | 20 | 20 | 20 |
| 88 | 0.461 | 8.6 | 20 | 20 | 20 |
| 89 | 0.054 | 20 | 20 | 20 | 20 |
| 90 | 0.014 | 20 | 20 | 20 | 20 |
| 91 | 0.054 | 20 | 20 | 20 | 20 |
| 92 | 0.996 | 20 | 20 | 20 | 18.339 |
| 93 | 0.497 | 20 | 20 | 20 | 20 |
| 94 | 1.013 | 20 | 20 | 20 | 20 |

It is to be understood that the invention is not limited to the particular embodiments of the invention described above, as variations of the particular embodiments may be made and still fall within the scope of the appended claims

1. A compound of formula (I): 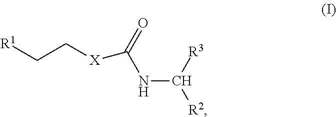
wherein:
X is triazole or imidazole;
R¹ is —NHC(═O)R⁴, —CH₂NHC(═O)R⁴, —NHC(═S)R⁴, —N═C(SCH₃)R⁴ or isoindolyl which is optionally substituted with oxo and/or C₁₋₇ alkoxy;
R² is —C(=O)OH, —C(═O)—C₁₋₇ alkoxy, —(CH₂)₂-phenyl, benzothiophenyl, naphthalenyl, benzodioxolyl, —C(═O)-methoxyphenyl, —CH₂C(═O)-phenyl, —(C(═O))₂NHCH₂-phenyl, phenyl or pyridinyl,
wherein said pyridinyl is optionally mono-substituted with C₁₋₇ alkyl or C₁₋₇ alkoxy, said phenyl is optionally mono- or bi-substituted independently with C₁₋₇ alkyl, —SCH₃, —CF₃, halogen, halo-C₁₋₇ alkyl, C₁₋₇ alkoxy, —OCH₂-phenyl or cyclopropyl;
R³ is hydrogen, cyano, —CH₂-indolyl or C₁₋₇ alkyl, said C₁₋₇ alkyl optionally substituted with phenyl;
R⁴ is phenyl, pyridinyl, pyridazinyl optionally mono-substituted with C₁₋₇ alkoxy, indenyl optionally mono-substituted with C₁₋₇ alkyl, —(CH₂)ₙ-dihydroindenyl optionally mono-substituted with hydroxy wherein n is 0 or 1, dihydrobenzofuranyl, dihydrobenzodioxinyl, isoindolinyl, benzofuranyl, or benzodioxinyl, wherein said phenyl is optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl,
wherein said pyridinyl is optionally mono- or bi-substituted independently with phenyl, C₁₋₇ alkoxy, —CF₃, —O-chlorophenyl, halogen or C₁₋₇ alkyl;
or a pharmaceutically acceptable salt thereof.

2. The compound according to claim 1, wherein X is triazole.

3. The compound according to claim 1, wherein R^{l} is —NHC(═O)R⁴.

4. The compound according to claim 1, wherein R² is —C(═O)OH, —C(═O)—C₁₋₇ alkoxy, —(CH₂)₂-phenyl, benzothiophenyl, naphthalenyl, benzodioxolyl, —C(═O)-methoxyphenyl, —CH₂C(═O)-phenyl or —(C(═O))₂NHCH₂-phenyl.

5. The compound according to claim 1, wherein R² is pyridinyl, optionally substituted with C₁₋₇ alkyl or C₁₋₇ alkoxy.

6. The compound according to claim 1, wherein R² is phenyl, optionally mono- or bi-substituted independently with C₁₋₇ alkyl, —SCH₃, —CF₃, halogen, C₁₋₇ alkoxy, —OCH₂-phenyl or cyclopropyl.

7. The compound according to claim 1, wherein R³ is hydrogen.

8. The compound according to claim 1, wherein R³ is cyano.

9. The compound according to claim 1, wherein R³ is C₁₋₇ alkyl, optionally substituted with phenyl.

10. The compound according to claim 1, wherein R⁴ is methoxyoxoisoindolinyl, methylindenyl, oxoisoindolinyl, benzodioxinyl, —CH₂-hydroxyindenyl, benzofuranyl, indenyl, —CH₂-indenyl, phenyl, said phenyl optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl, pyridinyl, said pyridinyl optionally mono- or bi-substituted independently with phenyl, C₁₋₇ alkoxy, —CF₃, —O-chlorophenyl, halogen or C₁₋₇ alkyl, or pyridazinyl, said pyridazinyl optionally substituted with C₁₋₇ alkoxy.

11. The compound according to claim 1, wherein R⁴ is methoxyoxoisoindolinyl, methylindenyl, oxoisoindolinyl, benzodioxinyl, —CH₂-hydroxyindenyl, benzofuranyl, indenyl, or —CH₂-indenyl.

12. The compound according to claim 1, wherein R⁴ is phenyl, optionally mono-, bi- or tri-substituted independently with C₁₋₇ alkoxy, halo-C₁₋₇ alkoxy, halogen, —SCH₃, —CF₃, C₁₋₇ alkyl, —SO₂CH₃ or C₁₋₇ alkoxy-C₁₋₇ alkyl.

13. The compound according to claim 1, wherein R⁴ is pyridinyl, optionally mono- or bi-substituted independently with phenyl, C₁₋₇ alkoxy, —CF₃, —O-chlorophenyl, halogen or C₁₋₇ alkyl.

14. The compound according to claim 1, wherein R⁴ is pyridazinyl, optionally substituted with C₁₋₇ alkoxy.

15. The compound according to claim 1, wherein said compound is:
1-[2-(2,3,4-Trimethoxy-benzoylamino)-ethyl]-1H-[1,2,3]triazole-4-carboxylic acid [cyano-(4-ethyl-phenyl)-methyl]-amide;
1-[2-(2,3,4-Trimethoxy-benzoylamino)-ethyl]-1H-[1,2,3]triazole-4-carboxylic acid [cyano-(4-methylsulfanyl-phenyl)-methyl]-amide;
1-[2-(2,3,4-Trimethoxy-benzoylamino)-ethyl]-1H-[1,2,3]triazole-4-carboxylic acid 4-methyl-benzylamide;
1-[2-(2,3,4-Trimethoxy-benzoylamino)-ethyl]-1H-[1,2,3]triazole-4-carboxylic acid 3,4-dimethyl-benzylamide;
1-[2-(2,3,4-Trimethoxy-benzoylamino)-ethyl]-1H-[1,2,3]triazole-4-carboxylic acid 4-chloro-benzylamide;
1-[2-(2,3,4-Trimethoxy-benzoylamino)-ethyl]-1H-[1,2,3]triazole-4-carboxylic acid 4-methylsulfanyl-benzylamide;
1-[2-(2,3,4-Trimethoxy-benzoylamino)-ethyl]-1H-[1,2,3]triazole-4-carboxylic acid 4-ethyl-benzylamide;
1-[2-(2,3,4-Trimethoxy-benzoylamino)-ethyl]-1H-[1,2,3]triazole-4-carboxylic acid [cyano-(4-methoxy-phenyl)-methyl]-amide;
1-[2-(2-Indan-2-yl-acetylamino)-ethyl]-1H-[1,2,3]triazole-4-carboxylic acid [cyano-(4-ethyl-phenyl)-methyl]-amide;
1-[2-(2,3,4-Trimethoxy-benzoylamino)-ethyl]-1H-[1,2,3]triazole-4-carboxylic acid [cyano-(4-trifluoromethoxy-phenyl)-methyl]-amide;
1-[2-(2-Methoxy-4-trifluoromethoxy-benzoylamino)-ethyl]-1H-[1,2,3]triazole-4-carboxylic acid [cyano-(4-ethyl-phenyl)-methyl]-amide;
1-[2-[(Indane-2-carbonyl)-amino]-ethyl}-1H-[1,2,3]triazole-4-carboxylic acid [cyano-(4-ethyl-phenyl)-methyl]-amide;
1-{2-[(2,3-Dihydro-benzofuran-7-carbonyl)-amino]-ethyl}-1H-[1,2,3]triazole-4-carboxylic acid [cyano-(4-ethyl-phenyl)-methyl]-amide;
1-[2-(2,5-Dichloro-benzoylamino)-ethyl]-1H-[1,2,3]triazole-4-carboxylic acid [cyano-(4-ethyl-phenyl)-methyl]-amide;
6-Phenyl-pyridine-2-carboxylic acid [2-(4-{[cyano-(4-ethyl-phenyl)-methyl]-carbamoyl}-[1,2,3]triazol-1-yl)-ethyl]-amide;
1-[2-(2,3-Dimethoxy-benzoylamino)-ethyl]-1H-[1,2,3]triazole-4-carboxylic acid [cyano-(4-ethyl-phenyl)-methyl]-amide;
3-Methoxy-pyridine-2-carboxylic acid [2-(4-{[cyano-(4-ethyl-phenyl)-methyl]-carbamoyl}-[1,2,3]triazol-1-yl)-ethyl]-amide;
1-{2-[(2,3-Dihydro-benzo[1,4]dioxine-5-carbonyl)-amino]-ethyl}-1H-[1,2,3]triazole-4-carboxylic acid [cyano-(4-ethyl-phenyl)-methyl]-amide;
1-[2-(2-Methoxy-4-methylsulfanyl-benzoylamino)-ethyl]-1H-[1,2,3]triazole-4-carboxylic acid [cyano-(4-ethyl-phenyl)-methyl]-amide;
1-[2-(4-Chloro-2-methoxy-benzoylamino)-ethyl]-1H-[1,2,3]triazole-4-carboxylic acid [cyano-(4-ethyl-phenyl)-methyl]-amide;
4-Chloro-6-methyl-pyridine-2-carboxylic acid [2-(4-{[cyano-(4-ethyl-phenyl)-methyl]-carbamoyl}-[1,2,3 ]triazol-1-yl)-ethyl]-amide;
1-{2-[2-(2-Hydroxy-indan-2-yl)-acetylamino]-ethyl}-1H-[1,2,3]triazole-4-carboxylic acid [cyano-(4-ethyl-phenyl)-methyl]-amide;
1-[2-(2-Methoxy-4-trifluoromethyl-benzoylamino)-ethyl]-1H-[1,2,3]triazole-4-carboxylic acid [cyano-(4-ethyl-phenyl)-methyl]-amide;
1-[2-(2-Trifluoromethoxy-benzoylamino)-ethyl]-1H-[1,2,3]triazole-4-carboxylic acid [cyano-(4-ethyl-phenyl)-methyl]-amide;
1-{2-[(3-Methyl-1H-indene-2-carbonyl)-amino]-ethyl]-1H-[1,2,3]triazole-4-carboxylic acid [cyano-(4-ethyl-phenyl)-methyl]-amide;
6-Methoxy-pyridine-2-carboxylic acid [2-(4-{[cyano-(4-ethyl-phenyl)-methyl]-carbamoyl}-[1,2,3]triazol-1-yl)-ethyl]-amide;
4-Chloro-pyridine-2-carboxylic acid [2-(4-{[cyano-(4-ethyl-phenyl)-methyl]-carbamoyl}-[1,2,3]triazol-1-yl)-ethyl]-amide;
5-Ethoxy-pyridine-2-carboxylic acid [2-(4-{[cyano-(4-ethyl-phenyl)-methyl]-carbamoyl}-[1,2,3]triazol-1-yl)-ethyl]-amide;
N-[2-(4-{[Cyano-(4-ethyl-phenyl)-methyl]-carbamoyl -[1,2,3]triazol-1-yl)-ethyl]-2-methoxy-nicotinamide;
5-Bromo-N-[2-(4-[cyano-(4-ethyl-phenyl)-methyl]-carbamoyl}-[1,2,3]triazol-1-yl)-ethyl]-4-methoxy-nicotinamide;
1-[2-(4-Chloro-2-fluoro-benzoylamino)-ethyl]-1H-[1,2,3]triazole-4-carboxylic acid [cyano-(4-ethyl-phenyl)-methyl]-amide;
1-[2-(2-Fluoro-5-methoxy-benzoylamino)-ethyl]-1H-[1,2,3]triazole-4-carboxylic acid [cyano-(4-ethyl-phenyl)-methyl]-amide;
1-[2-(2-Methoxymethyl-benzoylamino)-ethyl]-1H-[1,2,3]triazole-4-carboxylic acid [cyano-(4-ethyl-phenyl)-methyl]-amide;
N-[2-(4-{[Cyano-(4-ethyl-phenyl)-methyl]-carbamoyl}-[1,2,3]triazol-1-yl)-ethyl]-2-methoxy-6-methyl-nicotinamide;
N-[2-(4-[Cyano-(4-ethyl-phenyl)-methyl]-carbamoyl}-[1 ,2,3]triazol-1-yl)-ethyl]-2,6-dimethoxy-nicotinamide;
1-[2-(4-Fluoro-2-methoxy-benzoylamino)-ethyl]-1H-[1,2,3]triazole-4-carboxylic acid [cyano-(4-ethyl-phenyl)-methyl]-amide;
1-[2-(2-Fluoro-5-trifluoromethyl-benzoylamino)-ethyl]-1H-[1,2,3]triazole-4-carboxylic acid [cyano-(4-ethyl-phenyl)-methyl]-amide;
1-[2-(2-Fluoro-4-trifluoromethyl-benzoylamino)-ethyl]-1H-[1,2,3]triazole-4-carboxylic acid [cyano-(4-ethyl-phenyl)-methyl]-amide;
1-[2-(2-Methoxy-5-trifluoromethyl-benzoylamino)-ethyl]-1H-[1,2,3]triazole-4-carboxylic acid [cyano-(4-ethyl-phenyl)-methyl]-amide;
6-Methoxy-pyridazine-3-carboxylic acid [2-(4-[cyano-(4-ethyl-phenyl)-methyl]-carbamoyl}-[1,2,3]triazol-1-yl)-ethyl]-amide;
1-[2-(2-Chloro-4-methanesulfonyl-benzoylamino)-ethyl]-1H-[1,2,3]triazole-4-carboxylic acid [cyano-(4-ethyl-phenyl)-methyl]-amide;
1-[2-(5-Methanesulfonyl-2-methoxy-benzoylamino)-ethyl]-1H-[1,2,3]triazole-4-carboxylic acid [cyano-(4-ethyl-phenyl)-methyl]-amide;
5-(4-Chloro-phenoxy)-pyridine-2-carboxylic acid [2-(4-{[cyano-(4-ethyl-phenyl)-methyl]-carbamoyl}-[1,2,3]triazol-1-yl)-ethyl]-amide;
5-Chloro-pyridine-2-carboxylic acid [2-(4-{[cyano-(4-ethyl-phenyl)-methyl]-carbamoyl}-[1,2,3]triazol-1-yl)-ethyl]-amide;
5-Trifluoromethyl-pyridine-2-carboxylic acid [2-(4-{[cyano-(4-ethyl-phenyl)-methyl]-carbamoyl}-[1,2,3]triazol-1-yl)-ethyl]-amide;
1-[2-(1-Oxo-1,3-dihydro-isoindol-2-yl)-ethyl]-1H-[1,2,3]triazole-4-carboxylic acid [cyano-(4-ethyl-phenyl)-methyl]-amide;
1-[2-(5-Methoxy-1-oxo-1,3-dihydro-isoindol-2-yl)-ethyl]-1H-[1,2,3]triazole-4-carboxylic acid [cyano-(4-ethyl-phenyl)-methyl]-amide;
1-[2-(2,3,4-Trimethoxy-benzoylamino)-ethyl]-1H-[1,2,3]triazole-4-carboxylic acid (benzo[b]thiophen-5-yl-cyano-methyl)-amide;
1-[2-(2,3,4-Trimethoxy-benzoylamino)-ethyl]-1H-[1,2,3]triazole-4-carboxylic acid (cyano-naphthalen-2-yl-methyl)-amide;
1-[2-(2,3,4-Trimethoxy-benzoylamino)-ethyl]-1H-[1,2,3 ]triazole-4-carboxylic acid [(R)-cyano-(4-ethyl-phenyl)-methyl]-amide;
1-[2-(2,3,4-Trimethoxy-benzoylamino)-ethyl]-1H-[1,2,3]triazole-4-carboxylic acid [(S)-cyano-(4-ethyl-phenyl)-methyl]-amide;
1-[2-(2,3,4-Trimethoxy-benzoylamino)-ethyl]-1H-[1,2,3]triazole-4-carboxylic acid [cyano-(3-methoxy-phenyl)-methyl]-amide;
1-[2-(2,3,4-Trimethoxy-benzoylamino)-ethyl]-1H-[1,2,3]triazole-4-carboxylic acid (benzo[1,3]dioxol-5-yl-cyano-methyl)-amide;
1-[2-(2,3,4-Trimethoxy-benzoylamino)-ethyl]-1H-[1,2,3]triazole-4-carboxylic acid [(4-bromo-phenyl)-cyano-methyl]-amide;
1-[2-(2,3,4-Trimethoxy-benzoylamino)-ethyl]-1H-[1,2,3 ]triazole-4-carboxylic acid [cyano-(3-methylsulfanyl-phenyl)-methyl]-amide;
1-[2-(2,3,4-Trimethoxy-benzoylamino)-ethyl]-1H-[1,2,3]triazole-4-carboxylic acid [(4-benzyloxy-phenyl)-cyano-methyl]-amide;
1-[2-(2,3,4-Trimethoxy-benzoylamino)-ethyl]-1H-[1,2,3]triazole-4-carboxylic acid (cyano-naphthalen-1-yl-methyl)-amide;
1-[2-(2,3,4-Trimethoxy-benzoylamino)-ethyl]-1H-[1,2,3]triazole-4-carboxylic acid [cyano-(4-cyclopropyl-phenyl)-methyl]-amide;
1-[2-(2,3,4-Trimethoxy-benzoylamino)-ethyl]-1H-[1,2,3]triazole-4-carboxylic acid [(2-bromo-phenyl)-cyano-methyl]-amide;
1-[2-(2,3,4-Trimethoxy-benzoylamino)-ethyl]-1H-[1,2,3]triazole-4-carboxylic acid [cyano-(4-trifluoromethyl-phenyl)-methyl]-amide;
1-[2-(2,3,4-Trimethoxy-benzoylamino)-ethyl]-1H-[1,2,3]triazole-4-carboxylic acid [cyano-(4-isopropyl-phenyl)-methyl]-amide;
1-[2-(2,3,4-Trimethoxy-benzoylamino)-ethyl]-1H-[1,2,3]triazole-4-carboxylic acid (cyano-p-tolyl-methyl)-amide;
1-[2-(2,3,4-Trimethoxy-benzoylamino)-ethyl]-1H-[1,2,3 ]triazole-4-carboxylic acid [cyano-(5-ethyl-pyridin-2-yl)-methyl]-amide;
1-[2-(2,3,4-Trimethoxy-b enzoylamino)-ethyl]-1H-[1,2,3 ]triazole-4-carboxylic acid [cyano-(4-propyl-phenyl)-methyl]-amide;
1-[2-(2,3,4-Trimethoxy-benzoylamino)-ethyl]-1H-[1,2,3]triazole-4-carboxylic acid [cyano-(6-methoxy-pyridin-3-yl)-methyl]-amide;
1-[2-(2,3,4-Trimethoxy-benzoylamino)-ethyl]-1H-[1,2,3]triazole-4-carboxylic acid (benzo[b]thiophen-6-yl-cyano-methyl)-amide;
1-[2-(2,3,4-Trimethoxy-benzoylamino)-ethyl]-1H-[1,2,3]triazole-4-carboxylic acid [cyano-(4-isopropoxy-phenyl)-methyl]-amide;
1-[2-(2,3,4-Trimethoxy-benzoylamino)-ethyl]-1H-[1,2,3]triazole-4-carboxylic acid [(4-chloro-phenyl)-cyano-methyl]-amide;
1-[2-(2,5-Dichloro-benzoylamino)-ethyl]-1H-[1,2,3]triazole-4-carboxylic acid [cyano-(4-trifluoromethyl-phenyl)-methyl]-amide;
1-{2-[(3-Methyl-1H-indene-2-carbonyl)-amino]-ethyl}-1H-[1,2,3]triazole-4-carboxylic acid [cyano-(4-trifluoromethyl-phenyl)-methyl]-amide;
1-[2-(2-Methoxy-4-trifluoromethoxy-benzoylamino)-ethyl]-1H-[1,2,3]triazole-4-carboxylic acid [cyano-(4-trifluoromethyl-phenyl)-methyl]-amide;
1-[2-(2-Methoxy-4-trifluoromethyl-benzoylamino)-ethyl]-1H-[1,2,3]triazole-4-carboxylic acid [cyano-(4-trifluoromethyl-phenyl)-methyl]-amide;
1-[2-(2-Fluoro-4-trifluoromethyl-benzoylamino)-ethyl]-1H-[1,2,3]triazole-4-carboxylic acid [cyano-(4-trifluoromethyl-phenyl)-methyl]-amide;
1-[2-(2-Fluoro-4-methoxy-benzoylamino)-ethyl]-1H-[1,2,3]triazole-4-carboxylic acid [cyano-(4-trifluoromethyl-phenyl)-methyl]-amide;
1-[2-(2-Fluoro-4-methyl-benzoylamino)-ethyl]-1H-[1,2,3]triazole-4-carboxylic acid [cyano-(4-trifluoromethyl-phenyl)-methyl]-amide;
1-[2-(2-Fluoro-4-trifluoromethyl-benzoylamino)-ethyl]-1H-[1,2,3 ]triazole-4-carboxylic acid [cyano-(4-cyclopropyl-phenyl)-methyl]-amide;
1-[2-(2,3,4-Trimethoxy-benzoylamino)-ethyl]-1H-[1,2,3]triazole-4-carboxylic acid [2-(4-methoxy-phenyl)-2-oxo-ethyl]-amide;
1-[2-(2-Fluoro-4-trifluoromethyl-benzoylamino)-ethyl]-1H-[1,2,3]triazole-4-carboxylic acid (3-oxo-3-phenyl-propyl)-amide;
1-[2-(2-Fluoro-4-trifluoromethyl-benzoylamino)-ethyl]-1H-[1,2,3]triazole-4-carboxylic acid (1-methyl-3-phenyl-propyl)-amide;
(S)-4-Phenyl-2-({1-[2-(2,3,4-trimethoxy-benzoylamino)-ethyl]-1H-[1,2,3 ]triazole-4-carbonyl}-amino)-butyric acid ethyl ester;
(S)-2-({1-[2-(2-Fluoro-4-trifluoromethyl-benzoylamino)-ethyl]-1H-[1,2,3 ]triazole-4-carbonyl}-amino)-4-phenyl-butyric acid ethyl ester;
(S)-2-(1-[2-(2-Fluoro-4-trifluoromethyl-benzoylamino)-ethyl]-1H-[1,2,3 ]triazole-4-carbonyl}-amino)-4-phenyl-butyric acid;
(S)-4-Phenyl-2-({1-[2-(2,3,4-trimethoxy-benzoylamino)-ethyl]-1H-[1,2,3 ]triazole-4-carbonyl}-amino)-butyric acid;
(S)-2-(1-[2-(2-Fluoro-4-trifluoromethyl-benzoylamino)-ethyl]-1H-[1,2,3 ]triazole-4-carbonyl}-amino)-3-(1H-indol-2-yl)-propionic acid methyl ester;
1-[3-(2-Methoxy-4-trifluoromethyl-benzoylamino)-propyl]-1H-[1,2,3 ]triazole-4-carboxylic acid [cyano-(4-ethyl-phenyl)-methyl]-amide;
1-[2-(2,3,4-Trimethoxy-thiobenzoylamino)-ethyl]-1H-[1,2,3]triazole-4-carboxylic acid [cyano-(4-ethyl-phenyl)-methyl]-amide;
N-[2-(4-{[Cyano-(4-ethyl-phenyl)-methyl]-carbamoyl}-[1,2,3]triazol-1-yl)-ethyl]-2,3,4-trimethoxy-thiobenzimidic acid methyl ester;
1-[2-(2,3,4-Trimethoxy-benzoylamino)-ethyl]-1H-[1,2,3]triazole-4-carboxylic acid ((S)-1-benzyl-2-benzylcarbamoyl-2-oxo-ethyl)-amide;
5-[2-(2,3,4-Trimethoxy-benzoylamino)-ethyl]-2H-[1,2,4]triazole-3-carboxylic acid [cyano-(4-ethyl-phenyl)-methyl]-amide;
5-[2-(2-Methoxy-4-trifluoromethyl-benzoylamino)-ethyl]-2H-[1,2,4]triazole-3-carboxylic acid [cyano-(4-ethyl-phenyl)-methyl]-amide;
5-[2-(2-Fluoro-4-trifluoromethyl-benzoylamino)-ethyl]-2H-[1,2,4]triazole-3-carboxylic acid [cyano-(4-ethyl-phenyl)-methyl]-amide;
1-[2-(2-Methoxy-4-trifluoromethyl-benzoylamino)-ethyl]-1H-imidazole-4-carboxylic acid [cyano-(4-ethyl-phenyl)-methyl]-amide;
1-[2-(2-Methoxy-4-trifluoromethyl-benzoylamino)-ethyl]-1H-imidazole-4-carboxylic acid [(4-bromo-phenyl)-cyano-methyl]-amide; or
1-[2-(2-Fluoro-4-trifluoromethyl-benzoylamino)-ethyl]-1H-imidazole-4-carboxylic acid [cyano-(4-ethyl-phenyl)-methyl]-amide; or
pharmaceutically acceptable salts thereof.

16. A pharmaceutical composition, comprising a therapeutically effective amount of a compound according to claim 1 and a pharmaceutically acceptable carrier.

17-20. (canceled)

21. A method for treating an inflammatory disease or disorder selected from rheumatoid arthritis, lupus and irritable bowel disease, comprising the step of administering a therapeutically effective amount of a compound according to claim 1 to a subject in need thereof.

22. (canceled)
